(19) United States
(12) Patent Application Publication
(10) Pub. No.: US 2006/0141456 A1
(43) Pub. Date: Jun. 29, 2006

(54) Methods and compositions for milieu-dependent binding of a targeted agent to a target

(76) Inventors: Cynthia Edwards, Palo Alto, CA (US); Judith A Fox, Palo Alto, CA (US); Christopher J Murray, Soquel, CA (US); Volker Schellenberger, Palo Alto, CA (US); Robert J Tressler, Palo Alto, CA (US)

Correspondence Address: GENENCOR INTERNATIONAL, INC.;ATTENTION: LEGAL DEPARTMENT, 925 PAGE MILL ROAD, PALO ALTO, CA, 94304, US

(21) Appl. No.: 10/514,516

(22) PCT Filed: Jun. 9, 2003
(86) PCT No.: PCT/US03/18200

§ 371 Date: Oct. 28, 2005

Related U.S. Application Data
(60) Provisional application No. 60/388,387, filed on Jun. 12, 2002.

(51) Int. Cl. C12Q 1/68

(52) U.S. Cl. 435/6

(57) ABSTRACT

The present invention provides methods and compositions for milieu-dependent binding of a targeted agent to a target, for example, for the milieu-dependent binding of a diagnostic or therapeutic molecule to a diseased, injured or infected organ, tissue or cell.

### FIELD OF THE INVENTION

The present invention provides methods and compositions for milieu-dependent binding of a targeted agent to a target, for example, for the milieu-dependent binding of a diagnostic or therapeutic molecule to a diseased, injured or infected organ, tissue or cell.

### BACKGROUND

Traditional therapeutic molecules circulate freely throughout the body of patients treated with them, exerting their pharmocological effects indiscriminately on a wide range of cells and tissues, until they are removed from circulation by the liver. This can cause serious side effects in the patient. The problem is particularly acute when the molecule is a highly toxic chemotherapeutic agent used to kill cancer cells or tumors, where the difference between an efficacious dose and an injurious, or even lethal, dose can be small. Thus, in recent years, researchers have attempted to develop compounds that specifically affect particular subsets of cells, tissues or organs in a patient. By preferentially affecting targeted cells, tissues or organs, the difference between an efficacious dose and an injurious dose can be increased, which in turn increases the opportunity for a successful treatment regimen and reduces the occurrence of side effects.

Most of these compounds target a particular tissue by preferentially binding a particular target molecule that is displayed by the tissue to be treated. In one approach, a therapeutic molecule is linked to an antibody or antibody fragment recognizing a tumor antigen. One version of this approach is antibody-directed enzyme prodrug therapy (ADEPT). See e.g., Xu et al., 2001, Clin Cancer Res. 7:3314-24.; Denny, 2001, Eur J Med Chem. 36:577-95. In ADEPT, the antibody or antibody fragment targeting a desired tissue is attached to an enzyme. The ADEPT conjugate is administered to the patient, then the prodrug. The prodrug circulates throughout the body of the patient, but causes few or no side effects because it is in its inactive form. However, the prodrug is converted into its active drug form by the ADEPT conjugate's enzyme. Because the ADEPT conjugate is localized to the target tissue, the prodrug is activated only in the vicinity of the target tissue. Thus, a relatively highly concentration of active drug is produced in the vicinity of the target tissue, allowing the drug to exert its therapeutic effects, but a relatively low concentration of the active drug is present in the rest of the body.

While existing targeted therapeutic molecules represent an improvement over previously-available untargeted molecules, their usefulness is inherently limited by the frustrating observation that most, if not all, potential target molecules are found not just on the target tissue, but also on other tissues. Consequently, even targeted molecules can cause collateral damage in the patient's body. In fact, because they concentrate their effects on the subset of tissues displaying the target molecule, the damage they inflict on those tissues can be particularly severe.

Thus, there is a need in the art for methods and compositions for discriminating between a target tissue and a non-target tissue, each of which displays a target molecule.

### SUMMARY OF THE INVENTION

The present invention provides methods and compositions relating to milieu-dependent targeted agents (MDTAs), i.e., agents that preferentially bind to a microtarget (e.g., an epitope) when it is present in one context, relative to when it is present in another context. Examples of MDTAs include diagnostic or therapeutic molecules that binds a microtarget on a target but not on a non-target tissue, e.g., on an unhealthy tissue but not on a healthy tissue, on a healthy tissue but not on an unhealthy tissue, on a first unhealthy tissue but not on a second unhealthy tissue, or on a first healthy tissue but not on a second healthy tissue.

In one aspect, the present invention provides a method of binding a MDTA to a microtarget, comprising contacting the microtarget with the MDTA under conditions that allow the microtarget to bind to the MDTA.

In another aspect, the present invention provides a method of binding a MDTA to at least one microtarget in a compartment, comprising manipulating the compartment and contacting the at least one microtarget with the MDTA under conditions that allow the at least one microtarget to bind the MDTA.

In another aspect, the present invention provides a method of selectively binding a MDTA to a target comprising contacting the target and a non-target with the MDTA, wherein the target and the non-target each comprise a microtarget, under conditions that allow the MDTA to preferentially bind the microtarget in the target over binding the microtarget in the non-target.

In another aspect, the present invention provides a method of selectively binding a MDTA to a target, comprising manipulating a compartment and contacting the target and a non-target with the MDTA, wherein the target and the non-target each comprise at least one microtarget in the compartment and wherein the manipulation allows the MDTA to preferentially bind the at least one microtarget in the target over binding the at least one microtarget in the non-target.

In another aspect, the present invention provides a method of directing a MDTA to a target comprising a microtarget, said method comprising the step of contacting the target with the MDTA under conditions that allow the MDTA to preferentially bind the microtarget in the target relative to binding of the microtarget in a non-target.

In another aspect, the present invention provides a method of directing a MDTA to a target comprising at least one microtarget in a compartment, the compartment also having a non-target comprising at least one non-target microtarget, said method comprising the steps of manipulating the compartment and contacting the target with the MDTA under conditions in the compartment that allow the MDTA to preferentially bind the at least one microtarget in the target relative to binding of the at least one non-target microtarget in the non-target.

In another aspect, the present invention provides a method of binding a MDTA to a target comprising contacting the target and a non-target with the DTA, wherein the target comprises a microtarget in a first milieu, the nontarget comprises the microtarget in a second milieu, and the first milieu is not identical to the second milieu, under conditions that allow the MDTA to bind the microtarget in the first milieu but not the microtarget in the second milieu.

In another aspect, the present invention provides a method of binding a MDTA to a target comprising contacting the target and a non-target with MDTA, wherein the target comprises a microtarget in a first milieu, the nontarget comprises the microtarget in a second milieu, and the first milieu is not identical to the second milieu, under conditions that allow the MDTA to preferentially bind the microtarget in the first milieu relative to binding the microtarget in the second milieu.

In another aspect, the present invention provides a method of detecting a diseased, injured or infected tissue from a subject comprising

contacting a tissue from the subject with a detectable MDTA that preferentially

binds the diseased, injured or infected tissue over the healthy tissue, and

detecting the binding of the detectable MDTA to the tissue from the subject,

wherein an increase in binding of the detectable MDTA to the tissue from the subject relative to the binding of the MDTA to healthy tissue indicates that the subject has a diseased, injured or infected tissue.

In another aspect, the present invention provides a method of detecting a diseased, injured or infected tissue from a subject comprising

manipulating a compartment having the tissue

contacting the tissue from the subject with a detectable MDTA that preferentially binds the diseased, injured or infected tissue over a healthy tissue and

detecting the binding of the detectable MDTA to the tissue from the subject,

wherein an increase in binding of the detectable MDTA to the tissue from the subject relative to the binding of the MDTA to healthy tissue indicates that the subject has a diseased, injured or infected tissue.

In one embodiment, the tissue from the subject is removed from the subject before it is contacted with the detectable MDTA. In another embodiment, the tissue from the subject is contacted with the detectable MDTA by administering the detectable MDTA to the subject.

In another aspect, the present invention provides a method of treating a tissue in need of treatment in a subject comprising administering to the subject a MDTA that preferentially binds a target in the tissue when it is in need of treatment relative to the target in the tissue when it is not in need of treatment, wherein the MDTA comprises a therapeutic molecule for treating the tissue in need of treatment.

In another aspect, the present invention provides a method of treating a tissue in need of treatment in a subject comprising a) manipulating a compartment having the tissue to facilitate better binding of the MDTA to the tissue and b) administering to the subject a MDTA that preferentially binds a target in the tissue when it is in need of treatment relative to the target in the tissue lichen it is not in need of treatment, wherein the MDTA comprises a therapeutic molecule for treating the tissue in need of treatment.

In one embodiment, the tissue in need of treatment is diseased, injured or infected. In a more particularly defined embodiment, the tissue is cancerous tissue. In another embodiment, the therapeutic molecule is a chemotherapeutic molecule. In another embodiment, the therapeutic molecule is a targeted enzyme that can activate a prodrug. In another embodiment, the therapeutic molecule carries a radioactive isotope. In another embodiment, the therapeutic molecule is a conjugate between a peptide or protein and a cytotoxic or cytostatic compound.

In another aspect, the present invention provides a method of identifying a MDTA, comprising contacting a microtarget in a first milieu and the microtarget in a second milieu with an agent, wherein the agent is a MDTA if it preferentially binds the microtarget in the first milieu over binding the microtarget in the second milieu.

In another aspect, the present invention provides a method of identifying a MDTA, comprising a) manipulating a compartment, the compartment containing at least one microtarget and having a first milieu, the manipulation creating a second milieu in the compartment, the second milieu being different from the first milieu and b) contacting the at least one microtarget in the first milieu and the at least one mircotarget in the second milieu with an agent, wherein the agent is a MDTA if it preferentially binds the at least one microtarget in either milieu over binding the at least one microtarget in the other milieu.

In another aspect, the present invention provides a method of identifying a MDTA, comprising
a) contacting a microtarget in a first milieu with an agent, and
b) determining the level of agent binding to the microtarget;

so that if the agent preferentially binds the microtarget in the first milieu relative to its binding to the microtarget in a second milieu then a MDTA is identified.

In another aspect, the present invention provides a method of identifying a MDTA, comprising
a) manipulating a compartment, the compartment containing at least one microtarget and having a first milieu, the manipulation creating a second milieu in the compartment, the second milieu being different from the first milieu
b) contacting the at least one microtarget in the first milieu with an agent, and
c) determining the level of agent binding to the at least one microtarget;

so that if the agent preferentially binds the at least one microtarget in the first milieu relative to its binding to the at least one microtarget in the second milieu then a MDTA is identified.

In another aspect, the present invention provides a method of identifying a MDTA comprising
a) contacting a microtarget in a first milieu with a modified form of an agent, wherein an unmodified form of the agent binds the microtarget in the first milieu and the microtarget in a second milieu about equally, and
b) selecting the modified form of the agent if it preferentially binds the microtarget in the first milieu relative to its binding of the microtarget in the second milieu,
wherein the selected modified form of the agent is the MDTA.

In another aspect, the present invention provides a method of identifying a MDTA comprising
a) manipulating a compartment, the compartment containing at least one microtarget and having a first milieu, the manipulation creating a second milieu in the compartment, the second milieu being different from the first milieu
b) contacting the at least one microtarget in the first milieu with a modified form of an agent, wherein an unmodified form of the agent binds the at least one microtarget in the first milieu and the microtarget in the second milieu about equally, and
c) selecting the modified form of the agent if it preferentially binds the at least one-microtarget in the first milieu relative to its binding of the at least one microtarget in the second milieu,
wherein the selected modified form of the agent is the MDTA.

In another aspect, the present invention provides a method of identifying a MDTA comprising
a) contacting a microtarget in a first milieu with a modified form of an agent, wherein an unmodified form of the agent preferentially binds the microtarget in the first milieu relative to binding the microtarget in a second milieu, and
b) selecting the modified form of the agent if its preference for binding the microtarget in the first milieu relative to its binding of the microtarget in the second milieu is greater than the preference of the unmodifed form of the agent for binding the microtarget in the first milieu relative to its binding the microtarget in the second milieu,
wherein the selected modified form of the agent is the MDTA.

In another aspect, the present invention provides a method of identifying a MDTA comprising
a) manipulating a compartment, the compartment containing at least one microtarget and having a first milieu, the manipulation creating a second milieu in the compartment, the second milieu being different from the first milieu
b) contacting the at least one microtarget in the first milieu with a modified form of an agent, wherein an unmodified form of the agent preferentially binds the at least one microtarget in the first milieu relative to binding the at least one microtarget in the second milieu, and
c) selecting the modified form of the agent if its preference for binding the at least one microtarget in the first milieu relative to its binding of the at least one microtarget in the second milieu is greater than the preference of the unmodifed form of the agent for binding the at least one microtarget in the first milieu relative to its binding the at least one microtarget in the second milieu, wherein the selected modified form of the agent is the MDTA.

In one embodiment, the MDTA is or comprises a peptide, a polypeptide, a protein, a fusion protein, an antibody, an antibody fragment, an ADEPT molecule, a targeted enzyme prodrug therapy (TEPT) molecule, a conjugate comprising a small molecule or a small molecule having an activity (e.g., a biological activity, a pharmaceutical activity, an enzymatic activity, an enzyme inhibiting activity, an enzyme activating activity, a protease activity, a toxic activity, a growth factor activity, a hormone activity, an antibiotic activity, an antiviral activity, a narcotic activity, a radioactive activity, or an analgesic activity). In another embodiment, the MDTA comprises a detectable label (e.g., a radioactive label, a fluorescent label, a light-emitting label, a colorimetric label, a magnetic label or a detectable epitope).

In another embodiment, the target is a cell, tissue or organ, a diseased cell, tissue or organ, an infected cell, tissue or organ, an injured cell, tissue or organ, a tumor cell, a protein, a glycoprotein, a polypeptide, a peptide, a cell-surface protein, or a cell-surface receptor.

In another embodiment, the microtarget comprises all or a part of the target, or a plurality of parts of the target. In a more particularly defined embodiment, the microtarget is all or part of an epitope, a protein, a glycoprotein, a polypeptide, a peptide, a peptide sequence within a protein or polypeptide, a cell-surface protein, a cell-surface receptor, a carbohydrate, a lipid or a tumor antigen (e.g., a carcinoembryonic antigen, p97, A33, or MUC-1).

In another embodiment, the milieu is the reaction conditions in which the MDTA contacts, binds or is bound to the microtarget. In a more particularly defined embodiment, the milieu is a solvent, solution or-buffer, the cytoplasm of a cell, the intraorganellar space of a cell (wherein the organelle is, e.g., an endosome, the nucleus, the endoplasmic reticulum, the Golgi apparatus, a secretory vesicle, a mitochondrion or a chloroplast), the extracellular environment of a cell (e.g., the extracellular matrix, the periplasmic space or the cell wall), or the cellular context of the cell (e.g., the tissue or organ in which the cell resides).

In another embodiment, a first and a second milieu differ from each other in a way that allows an MDTA to preferentially bind a microtarget in the first milieu relative to the second milieu, e.g., under conditions that allow binding fo the microtarget in the first milieu, but not in the second milieu. The difference between the first and second milieu that allows the MDTA to preferentially bind the microtarget in the first milieu relative to the second milieu can be known or unknown to the operator; that is, the mechanism for differential binding need not be known. In a more particularly defined embodiment, the difference between the first and second milieu is a difference in pH, the partial pressure of a gas (e.g., O₂ or CO₂), temperature, osmolarity, salt concentration, concentration of a solute (e.g., lactic acid, sugars or other organic or inorganic molecules), ionic strength or light.

All references are incorporated herein by reference in their entireties for all purposes.

### BRIEF DESCRIPTION OF THE FIGURES

FIG. 1 presents a schematic diagram of plasmid pADEPT06. P lac=lac promoter, Pel B leader sequence=signal seq, L49VH=Heavy chain, L49VL=Light chain, 218 linker=linker region between heavy and light chains, β-lactamase=β-lactamase gene, L49sFv-bl=scFv-BLA fusion, CAT=chloramphenicol resistance gene.

FIG. 2 presents the amino acid sequence of a β-lactamase.

FIG. 3 presents a graph demonstrating how the timing of the administration of an MDTA and of a prodrug to a subject can be manipulated to improve the specificity of the treatment for a target tissue.

FIG. 4 illustrates an embodiment of the invention for selecting pH-dependent binding sequences.

FIG. 5 shows results of a secondary screening of 21 mutants in quadruplicates. The x-axis shows variant designation and the y-axis shows the performance index. A ratio of bound activity at T₁ vs. T₀ was calculated for each mutant, and an index was calculated by dividing the ratio of mutant over parent, as shown in Table 3.

FIG. 6 present details related to plasmid pME27.1 FIG. 6A presents a schematic diagram of plasmid pME27.1. P lac=lac promoter, Pel B leader sequence=signal seq, CAB1scFN-single chain antibody, BLA=β-lactamase gene, CAT=chloramphenicol resistance gene, T7 terminator=terminator. FIG. 6B presents shows the sequence of CAB1-scFv, the CDRs and mutations chosen for combinatorial mutagenesis. FIG. 6C presents and nucleotide sequence of pME27.1 FIG. 6D shows the amino acid sequence of CAB1 which shows, for example, the sequence of the heavy chain, the sequence of the linker, the sequence of the light chain and the sequence of BLA.

FIG. 7 shows binding assays and SDS page results. Specifically, FIG. 7A shows the binding of variants from library NA05: FIG. 7B displays and SDS PAGE of stable CAB1-BLA variants of the NA05 library; FIG. 7C shows binding of various isolates from NA06 to CEA.

FIG. 8 shows a comparison of vH and vL sequences of CAB1-scFv with a published frequency analysis of human antibodies. Specifically, FIG. 8A shows the observed frequencies of the five most abundant amino acids in alignment of human sequence in the heavy chain; FIG. 8B shows the observed frequencies of the five most abundant amino acids in alignment of human sequence in the light chain.

FIG. 9 shows screening results of NA08 library. The x-axis shows binding at pH 7.4, and the Y-axis shows binding at pH 6.5. Clones that were chosen are represented by a square.

FIG. 10 shows positions that were chosen for combinatorial mutagenesis.

FIG. 11 shows pH-dependent binding of NA08 variants to immobilized carcinoembryonic antigen.

### DETAILED DESCRIPTION OF THE INVENTION

The present invention provides methods and compositions relating to milieu-dependent targeted agents (MDTAs), i.e., agents that preferentially bind to a microtarget (e.g., a molecule or part of a molecule) when it is present in one context, relative to when it is present in another context. Examples of MDTAs include, e.g., diagnostic or therapeutic molecules that preferentially bind to a microtarget present on one type of cell, tissue or organ over binding to the microtarget when it is present on a different cell, tissue or organ.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are used as described below.

A “milieu-dependent targeted agent” (MDTA) is a targeted agent that preferentially binds to a microtarget present on a target relative to binding of the microtarget present on a non-target. The difference in binding can be caused by any difference between the target and non-target such as, for example, a difference in pH, oxygen pressure, concentration of solutes or analytes (e.g., lactic acid, sugars or other organic or inorganic molecules), temperature, light or ionic strength.

A “target” is a composition or surface comprising at least one microtarget.

A “microtarget” is the chemical structure or surface that a targeted agent binds to, including, for example, all or part of, or multiple parts of, one or more molecules.

A “tumor antigen” is a microtarget that is expressed in higher abundance in tumor tissue as compared to most other tissues.

A “compartment” is a region containing a target or a region or system affecting the target, even though a compartment may be physically separate from the compartment. Compartments may include, for example, without limitation, the CNS, organs, the lymphatic system or specific drainage regions of the lymphatic system, the cardiopulmonary system, the skeletal network or distinct regions of the skeletal region, endocrine regions or specific regions of the endocrine regions and/or other molecular networks or activation cascades for inflammatory mediators, cytokines, growth factors and/or hydrolases.

A “targeted agent” is a chemical entity that binds selectively to a microtarget of interest. Examples of targeted agents are antibodies, peptides and inhibitors. Of particular interest are fusion proteins between antibodies or antibody fragments and enzymes. Also of interest are targeted enzymes that have a desired catalytic activity and that can bind to one or more target structures with high affinity and selectivity. Targeted enzymes retain at least most of their activity while bound to a target.

A “binding moiety” is a part of a targeted agent that binds a microtarget. A binding moiety can comprise more than one region, either contiguous or non-contiguous, of the MDTA.

An “active moiety” is a part of a MDTA that confers a functionality to the MDTA. An active moiety can comprise more than one region, either contiguous or non-contiguous, of the MDTA.

37 Manipulate” and “manipulating” shall mean an external imposition, as opposed to intrinsic, upon conditions that modify (e.g., augment or diminish) a target. Conditions that constitute an imposition on a compartment may include, for example, alterations in pH, interstitial tonicity, blood flow, interstitial fluid flow, temperature or other physical or chemical alterations or additions to augment or diminish a target or antitarget compartment.

“Physiological conditions” are conditions that are identical to, similar to, or compatible with the conditions found inside a living organism, e.g., a human being. The physiological condition can be associated with a healthy or normal state, or with a diseased, injured or infected state. Examples of physiological conditions include those found in the bloodstream, in the interstitial spaces within or between tissues, organs or cells (e.g., the interstitial spaces of a tumor), and inside of cells.

Unless otherwise noted, the term “protein” is used interchangeably here with the terms “peptide” and “polypeptide,” and refers to a molecule comprising two or more amino acid residues joined by a peptide bond.

The terms “cell”, “cell line”, and “cell culture” can be used interchangeably and all such designations include progeny. Thus, the words “tansformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants. The cells can be prokaryotic or eukaryotic.

The term “control sequences” refers to DNA sequences necessary for the expression of an operably linked coding sequence in a particular host organism. The control sequences that are suitable for procaryotes, for example, include a promoter, optionally an operator sequence, a ribosome binding site, positive retroregulatory elements (see, e.g., U.S. Pat. No. 4,666,848, incorporated herein by reference), and possibly other sequences. Eucaryotic cells are known to utilize promoters, polyadenylation signals, and enhancers.

The term “expression clone” refers to DNA sequences containing a desired coding sequence and control sequences in operable linkage, so that hosts transformed with these sequences are capable of producing the encoded proteins. The term “expression system” refers to a host transformed with an expression clone. To effect transformation, the expression clone may be included on a vector; however, the relevant DNA may also be integrated into the host chromosome.

The term “gene” refers to a DNA sequence that comprises control and coding sequences necessary for the production of a protein, polypeptide or precursor.

The term “operably linked” refers to the positioning of the coding sequence such that control sequences will function to drive expression of the protein encoded by the coding sequence. Thus, a coding sequence “operably linked” to control sequences refers to a configuration wherein the coding sequences can be expressed under the direction of a control sequence.

The term “oligonucleotide” as used herein is defined as a molecule comprised of two or more deoxyribonucleotides or ribonucleotides. The exact size will depend on many factors, which in turn depends on the ultimate function or use of the oligonucleotide. Oligonucleotides can be prepared by any suitable method, including, for example, cloning and restriction of appropriate sequences and direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066, each incorporated herein by reference. A review of synthesis methods is provided in Goodchild, 1990, Bioconjugate Chemistry 1(3):165-187, incorporated herein by reference.

The term “primer” as used herein refers to an oligonucleotide which is capable of acting as a point of initiation of synthesis when placed under conditions in which primer extension is initiated. Synthesis of a primer extension product that is complementary to a nucleic acid strand is initiated in the presence of the requisite four different nucleoside triphosphates and a DNA polymerase in an appropriate buffer at a suitable temperature. A “buffer” includes cofactors (such as divalent metal ions) and salt (to provide the appropriate ionic strength), adjusted to the desired pH.

A primer that hybridizes to the non-coding strand of a gene sequence (equivalently, is a subsequence of the coding strand) is referred to herein as an “upstream” or “forward” primer. A primer that hybridizes to the coding strand of a gene sequence is referred to herein as an “downstream” or “reverse” primer.

The terms “restriction endonucleases” and “restriction enzymes” refer to enzymes, typically bacterial in origin, which cut double-stranded DNA at or near a specific nucleotide sequence.

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine), nonpolar side chains (e.g., alanine, valine, leucine, isoleucine, proline, phenylalanine, methionine, tryptophan, cysteine, glycine), beta-branched side chains (e.g., threonine, valine, isoleucine) and aromatic side chains (e.g., tyrosine, phenylalanine, tryptophan, histidine). Standard three-letter or one-letter amino acid abbreviations are used herein.

The peptides, poly,peptides and proteins of the invention can comprise one or more non-classical amino acids. Non-classical amino acids include but are not limited to the D-isomers of the common amino acids, a-amino isobutyric acid, 4-aminobutyric acid (4-Abu), 2-aminobutyric acid (2-Abu), 6-amino hexanoic acid (Ahx), 2-amino isobutyric acid (2-Aib), 3-amino propionoic acid, ornithine, norleucine, nonraline, hydroxyproline, sarcosine, citrulline, cysteic acid, t-butylglycine, t-butylalanine, phenylglycine, cyclohexylalanine, B-alanine, fluoro-amino acids, designer amino acids such as B-methyl amino acids, Ca-methyl amino acids, Nα-methyl amino acids, and amino acid analogs in general.

As used herein, a “point mutation” in an amino acid sequence refers to either a single amino acid substitution, a single amino acid insertion or single amino acid deletion. A point mutation preferably is introduced into an amino acid sequence by a suitable codon change in the encoding DNA. Individual amino acids in a sequence are represented herein as AN, wherein A is the standard one letter symbol for the amino acid in the sequence, and N is the position in the sequence. Mutations within an amino acid sequence are represented herein as A₁ NA₂, wherein A₁ is the standard one letter symbol for the amino acid in the unmutated protein sequence, A₂ is the standard one letter symbol for the amino acid in the mutated protein sequence, and N is the position in the amino acid sequence. For example, a G46D mutation represents a change from glycine to aspartic acid at amino acid position 46. The amino acid positions are numbered based on the full-length sequence of the protein from which the region encompassing the mutation is derived. Representations of nucleotides and point mutations in DNA sequences are analogous.

As used herein, a “chimeric” protein refers to a protein whose amino acid sequence represents a fusion product of subsequences of the amino acid sequences from at least two distinct proteins. A chimeric protein preferably is not produced by direct manipulation of amino acid sequences, but, rather, is expressed from a “chimeric” gene that encodes the chimeric amino acid sequence.

The term “Ab” or “antibody” refers to polyclonal, monoclonal antibodies, chimeric antibodies, humanized antibodies, human antibodies, immunoglobulins, or antibody or functional fragment of an antibody that binds to a target antigen. Examples of such functional entities include complete antibody molecules, antibody fragments, such as Fv, single chain Fv, complementarity determining regions (CDRs), V_{L} (light chain variable region), V_{H} (heavy chain variable region), and any combination of those or any other functional portion of an immunoglobulin peptide capable of binding to target antigen.

The terms “dox” and “doxorubicin” refer to the drug commonly known by that name and any derivative thereof Derivatives may be made for a variety of purposes including, but not limited to, conjugating to a linker or pro-part of a prodrug, increased efficacy, increased binding, decreased toxicity, etc. The CAS Registry Number for Doxorubicin is 25316409. The molecular formula is C₂₇H₂₉NO₁₁.HCl and its molecular weight is 580 Daltons.

The term “PEG” and polyethylene glycol” refer to the compounds commonly known by the name and comprising the general chemical formula (C₂H₄O)ₙ.H₂O. The CAS Number for PEG is 25322-68-3. As is well known in the art, PEG is typically provided in mixtures of differing molecular weights. For example, PEG-8000 is a mixture of polyethylene glycols that have an average molecular weight of 8,000 Daltons.

The term “prodrug” refers to a compound that is converted via one or more enzymatically catalyzed steps into an active compound that has an increased pharmacological activity relative to the prodrug. A prodrug can comprise a pro-part or inactive moiety and a drug or active drug. Optionally, the prodrug also contains a linker. For example, the prodrug can be cleaved by an enzyme to release an active drug. In a more specific example, prodrug cleavage by the targeted enzyme releases the active drug into the vicinity of the target bound to the targeted enzyme. “Pro-part” and “inactive moiety” refer to the inactive portion of the prodrug after it has been converted. For example, if a prodrug comprises PEG molecule linked by a peptide to an active drug, the pro-part is the PEG moiety with or without a portion of the peptide linker. “Linker” refers to the means connecting the pro-part of a prodrug to the active drug of a prodrug. Typically, but not essentially, the linker is a peptide cleavable by the targeted enzyme, however, it can be any moiety that joins the drug to the propart. The term “drug” and “active drug” refer to the active moieties of a prodrug. After cleavage by a targeted enzyme, the active drug acts therapeutically upon the targeted tumor, cell, infectious agent or other agent of disease. In another example, the prodrug is chemically modified by the activating enzyme, for example, by oxidation, reduction, phosphorylation, dephosphorylation, the addition of a moiety, or the like. In another example, the prodrug is converted into an intermediate compound by the enzyme. The intermediate compound is converted to the active compound either spontaneously, through contact with other proteins or molecules in the subject, through contact with one or more enzymes native to the subject, or through contact with one or more additional activating enzymes administered to the subject.

The term “serum albumin” refers to the commonly known blood protein of the same name. “BSA” refers to bovine serum albumin and “HSA” refers to human serum albumin.

The term “% sequence homology” is used interchangeably herein with the terms “% homology,” “% sequence identity” and “% identity” and refers to the level of amino acid sequence identity between two or more peptide sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 60, 70, 80, 85, 90, 95, 98 or 99% or more sequence identity to a given sequence

Exemplary computer programs which can be used to determine identity between two sequences include, but are not limited to, the suite of BLAST programs, e.g., BLASTN, BLASTX, and TBLASTX, BLASTP and TBLASTN, which are well-known to one skilled and the art and may publicly available on the Internet at http://www.ncbi.nim.nib.pov/BLAST/”. See also Altschul et al., 1990, J. Mol. Biol. 215: 403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See Altschul, et al., 1997.

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

In one aspect the present invention provides a milieu-dependent targeted agent (MDTA). An MDTA is a targeted agent that preferentially binds to a microtarget present on a target relative to binding of the microtarget present on a non-target. The difference in binding can be caused by any difference between the target and non-target such as, for example, a difference in pH, oxygen pressure, concentration of solutes or analytes (e.g., lactic acid, sugars or other organic or inorganic molecules), temperature, light or ionic strength. From the following, it will be clear to one of skill in the art that the MDTAs of this invention have many uses. For example, they can be used to bind to a microtarget under a desired set of conditions, identify a target in vitro, ex vivo, in situ or in vivo (e.g., a target tissue in a subject), kill a target cell or tissue, convert a prodrug into an active drug in or near a target tissue. They also can be used as surface catalysts, for example, a targeted laccase. Other uses include, e.g., targeted generation of a compound (e.g., H₂O₂ from glucose) and the targeted destruction of compounds (e.g., a metabolite or signalling molecule from a particular tissue).

In one embodiment, the MDTA is selected, made or modified using an affinity maturation method. e.g., as described in U.S. Pat. App. Ser. No. 60/388,386 (attorney docket no. 9342-040-999), filed concurrently with the present application, incorporated herein by reference in its entirety.

In another embodiment, the MDTA is selected, made or modified using a loop-grafting method, e.g., as described in U.S. Pat. App. Ser. No. 60/279,609 (attorney docket no. 9342-014-999), filed concurrently with the present application, incorporated herein by reference in its entirety.

In another embodiment, the MDTA is a multifunctional polypeptide, e.g., as described in U.S. Pat. App. Ser. No. 10/170,729 (attorney docket no. 9342-043-999), filed concurrently with the present application, incorporated herein by reference in its entirety.

Binding of MDTA to Microtarget

MDTAs of the invention include MDTAs that bind to a microtarget better under one set of reaction conditions than under another set of reaction conditions. For example, the present invention provides an MDTA that binds a microtarget under one set of reaction conditions with about 2-fold, 3-fold, 5-fold, 10-fold, 20-fold, 10²-fold, 10³-fold, 10⁴-fold, 10⁵-fold, 10⁶-fold or higher affinity than under another set of reaction conditions. Under conditions allowing the MDTA to bind to the microtarget, the MDTA can, for example, bind to the microtarget with a K_{d} of about 100 μM or less, 10 μM or less, 1 μM or less, 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 5 nM or less, about 1 nM or less or about 0.1 nM or less.

Size of MDTA

In one aspect, the MDTAs of the invention are used for diagnostic or therapeutic administration. It is known that macromolecules with molecular weights below about 45,000 Daltons are rapidly cleared from the circulation by glomerular filtration of the kidney. See also Greenwald et al., Crit Rev Ther Drug Carrier Syst 17:101 (2000). In one embodiment, therefore, the present invention provides a MDTA that has a molecular weight that allows its removal from the circulation of a mammalian host via glomerular filtration. It is noted that in addition to having a shorter half-life in the circulation, smaller MDTAs diffuse more quickly into certain types of targets, e.g., a tumor mass. For in vivo applications, MDTAs also are preferred that have a relatively small size, preferably smaller than about 45 kD, and that are subject to minimal interference in the treated subject from, for example, inhibitors, enzyme substrates, or endogenous enzyme systems.

In other aspects, the MDTA has a molecular weight greater than 5 kD but less than 10 kD, 15 kD, 20 kD, 25 kD, 30 kD, 35 kD, 40 kD, 45 kD, 50 kD, 55 kD or 60 kD, 75 kD, 100 kD, 150 kD, 200 kD, 250 kD, 300 kD, 350 kD, 400 kD, 450 kD or 500 kD.

Binding Moiety of MDTA

In another aspect, the present invention provides an MDTA comprising a binding moiety that preferentially binds to a microtarget under one set of reaction conditions relative to its binding to the microtarget under a second set of reaction conditions. The binding moiety can comprise any type of molecule. Examples of types of molecules that the binding moiety can comprise include, for example, a peptide, polypeptides, proteins, antibodies, antibody fragments, single chain antibody variable region fragment (scFv), ligand-binding peptides, polypeptides or proteins, receptor-binding peptides, polypeptides or proteins, organic molecules (e.g., sugars, amino acids, nucleotides or small organic molecules) or inorganic molecules. The binding moiety can be, for example, the scFv molecules SGN17, CAB or TAG-72. In one embodiment, the binding moiety is not identical to a binding domain found in a naturally-occurring protein (e.g., the neonatal Fc receptor/immunoglobulin G binding binding domains, see Raghaven et al., 1995, Biochemistry 34:14649-57, incorporated herein by reference in its entirety). The binding moiety of the MDTA can comprise more than one molecule or portion of the MDTA, for example, two or more non-contiguous peptide sequences within the MDTA can comprise the binding moiety, e.g., as in a TEPT molecule, as described below.

In one embodiment, the binding moiety of an MDTA is derived from a prototype binding moiety that binds the microtarget under a first and a second set of reactions conditions. Variants of the prototype binding moiety are screened for variants with improved binding for the microtarget under the first set of reaction conditions and/or reduced binding for the microtarget under the second set of reaction conditions, such that the variant preferentially binds to the microtarget under the first set of reaction conditions as compared to binding to the microtarget under the second set of reaction conditions.

In one embodiment, the binding moiety of the MDTA preferentially binds a microtarget at one pH relative to another. For example, in one embodiment, the MDTA preferentially binds a microtarget at a lower pH than at a higher pH. In another embodiment, the lower pH is the pH of the interstitial spaces of a cancerous tissue and the higher pH is the interstitial spaces of a healthy tissue. As the difference between these pH values can be small (typically representing a difference in proton concentration of 10-fold or less), the binding moiety for this embodiment is sensitive to small changes in pH. Thus, in another embodiment, the binding moiety has a binding affinity that changes at least 5 or 10-fold in response to a pH change of 0.7 units or less. A binding moiety can be made sensitive to pH by, for example, incorporating one or more ionizable groups. In one embodiment, the ionizable groups are ionizable groups with pK values that are similar to the pH in a milieu of interest. In another embodiment, the ionizable groups are ionizable groups with pK values between the pH value of the target's milieu and the pH value of the non-target's milieu. In one embodiment, the group comprises one or more negative charges that can interact with the ionizable groups of the microtarget.

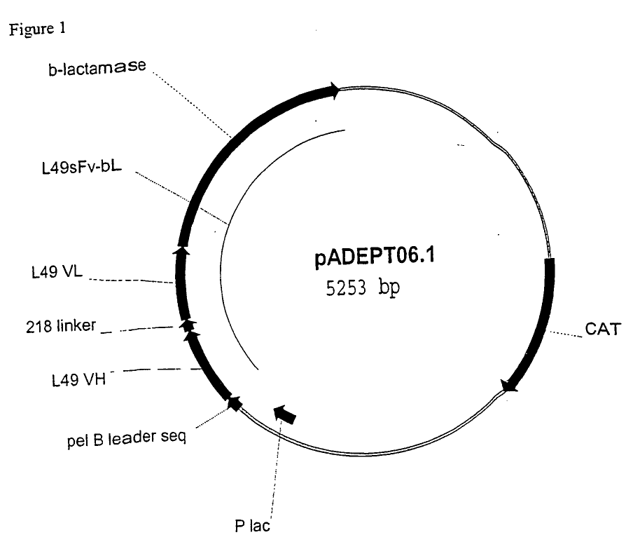

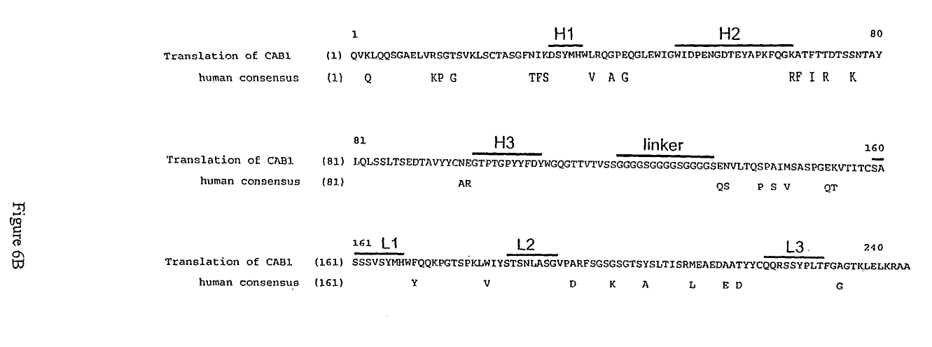

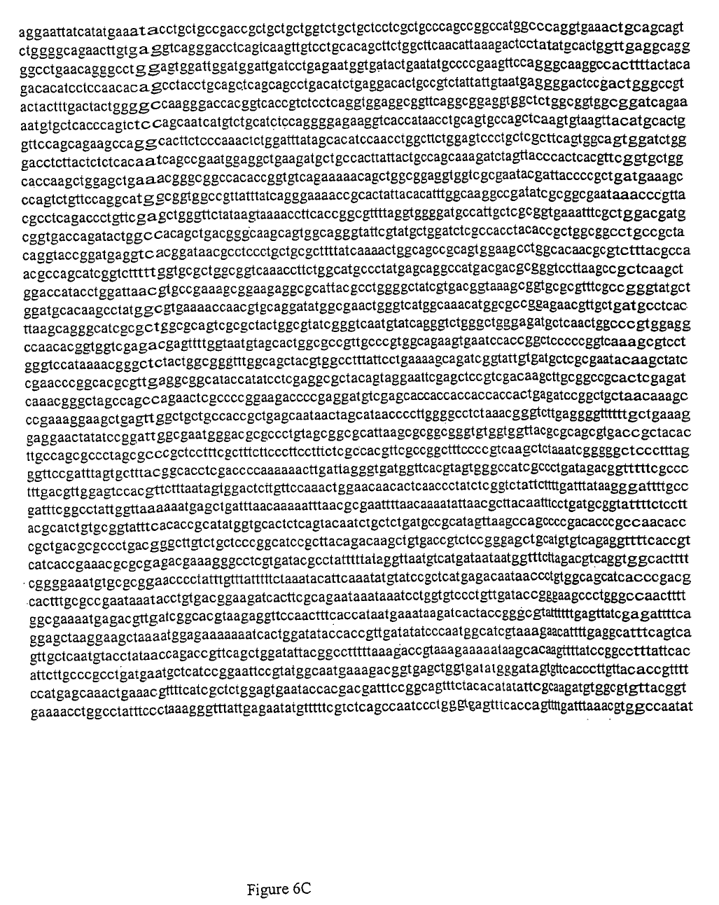

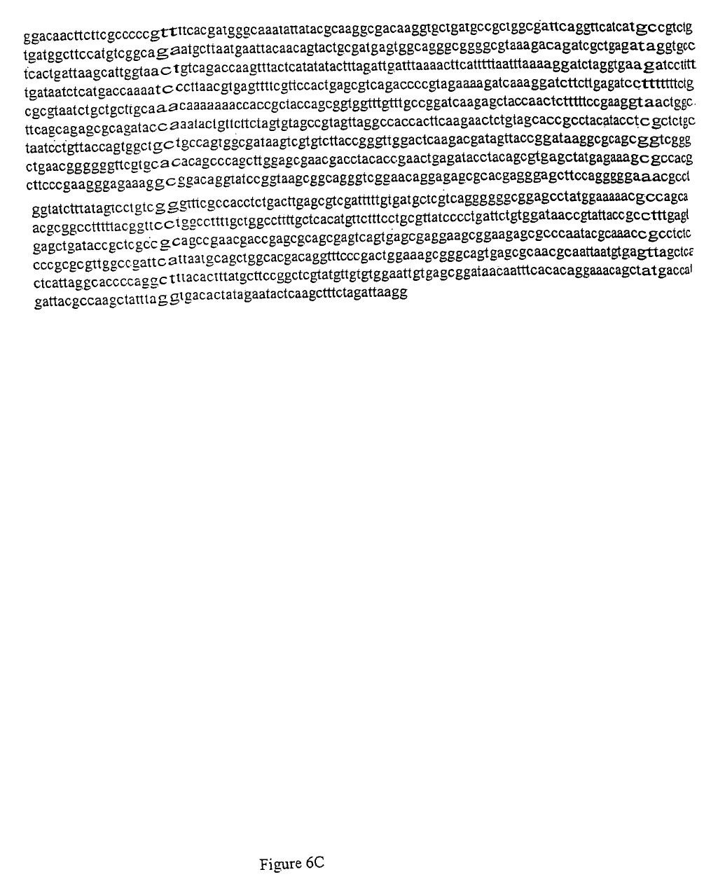

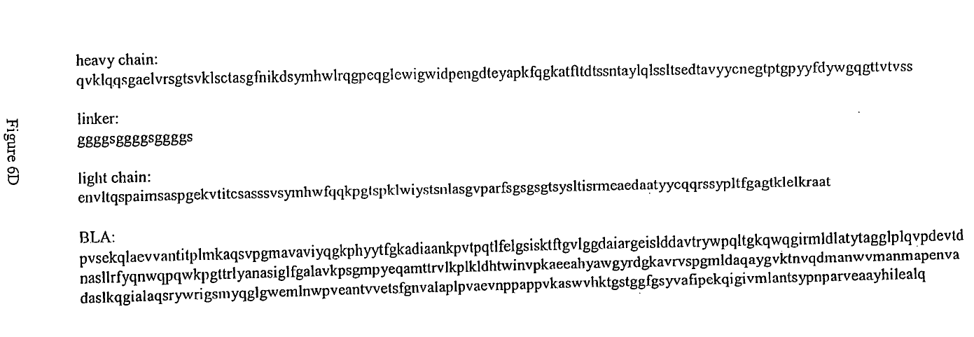

In one embodiment, the binding molecule comprises any of the amino acid sequences of FIG. 1 or FIG. 6. In another embodiment, the binding molecule is at least 50%, 60%, 70%, 80%, 90%, 95%, 98% or 99% or more identical to the sequence depicted in FIG. 1 or FIG. 6.

In another aspect, the MDTA comprises more than one binding moiety, for example, two or more identical binding moieties, two or more different binding moieties that each preferentially binds the same microtarget under the same reaction conditions, two or more different binding moieties that each preferentially binds the same microtarget under different reaction conditions, two or more different binding moieties that each preferentially binds a different microtarget under the same reaction conditions, or two or more binding moieties that each preferentially binds a different microtarget under different reaction conditions.

By combining in one MDTA more than one identical binding moieties, or different binding moieties that each binds to the same microtarget under the same reaction conditions, the MDTA can be more precisely targeted. By combining two or more different binding moieties that each binds the same microtarget under different reaction conditions, an MDTA can be designed that binds two different targets, e.g., the same microtarget in two or more different tissues from a subject. By combining in one MDTA two or more different binding moieties that each binds a different microtarget under the same reaction conditions, an MDTA can be designed that, for example, simultaneously binds two different microtargets on the same target, thereby increasing the specificity of binding of the MDTA. Alternatively, each of the microtargets can be on a different target, e.g., on two different tissues from a subject. By combining in one MDTA two different binding moieties that each binds a different microtarget under different conditions, an MDTA can be designed that binds one target under one set of reaction conditions and another target under another set of reaction conditions.

Active Moiety

In one aspect, the present invention provides an MDTA comprising an active moiety. The active moiety can be a molecule, or a part of a molecule, that has an activity. The activity can be any activity. Examples of types of activities that the active moiety can have include, for example, a detectable activity, an enzymatic activity, a therapeutic activity, a diagnostic activity, a toxic activity, or a binding activity. The active moiety can be a discrete part of the MDTA, for example, an enzyme that is fused or conjugated to the binding moiety, or it can be an integral part of the MDTA, for example, binding of the MDTA to the microtarget can activate or inhibit an activity of the microtarget or the target, or the MDTA can be a targeted enzyme of the type discussed below and in copending U.S. patent application Ser. Nos. 10/022,073 and 10/022,097, incorporated herein by reference in their entireties.

In one embodiment, the MDTA comprises an active moiety and a binding moeity that are not part of the same naturally-occurring protein.

In one embodiment, the active moiety of an MDTA is more active in a first milieu than in a second milieu, wherein the MDTA preferentially binds to a microtarget in the first milieu over binding to the microtarget in the second milieu. Such a combination of a milieu-dependent binding moiety and a milieu-dependent active moiety allows for the design of an MDTA with an increased specificity for its target. In one embodiment, the MDTA comprises multimerization domains that allow it to form aggregates in dependence of the milieu and thus aggregate at the target. The aggregation can be further enhanced by interactions of the MDTA with its microtarget.

In one embodiment, the active moiety is a detectable moiety. Detection can be either direct or indirect. Examples of ways in which the detectable moiety can be detected include, for example, being bound by an antibody, being bound by a protein, being bound by another molecule (e.g., a His tag that is bound by a nickel column, a nucleic acid that hybridizes to a complementary sequence), emitting light, fluorescing or emitting radioactivity.

In another embodiment, the active moiety is a toxic moiety. The toxic moiety can any toxic molecule. Examples of toxic moieties include radioactive groups, ricin, diphtheria toxin, Pseudomonas exotoxin, gelonin, and doxorubicin.

In another embodiment, the active moiety comprises a nucleic acid to be delivered to a target cell, e.g., for the purposes of gene therapy, antisense therapy or ribozyme therapy. The nucleic acid can be any type of nucleic acid and have any sequence of nucleotides. The nucleic acid-can be, for example, DNA, RNA, or a synthetic or artificial nucleic acid, such as a peptide nucleic acid, or a combination of any types of nucleic acid. The nucleic acid can, for example, encode a protein or peptide (e.g., that provides an enzymatic activity to the cell, or kills the cell), or it can be an antisense RNA a ribozyme structure. The nucleic acid can be, for example, incorporated into the target cell's genome (e.g., through homologous or non-homologous recombination).

In another embodiment the active moiety exhibits enzymatic activity, e.g., it is an enzyme or an active fragment or derivative of an enzyme. The enzyme can be any enzyme. Examples of enzymes that can be used include an enzyme that is active in diseased cells with altered physiological states, for example, in cancer cells with lowered pH. Of particular interest are enzymes that can be used to activate a prodrug in a therapeutic setting. A large number of enzymes with different catalytic modes of action have been used to activate prodrugs. See, e.g., Melton & Knox Enzyme-prodrug strategies for cancer therapy (1999) and Bagshawe et al., Curr Opin. Immunol 11:579 (1999). These enzymes can be modified to incorporate milieu-dependent targeting capability into the protein (e.g., by fusing or conjugating the enzyme to a MDTA, or by creating a milieu-dependent targeted enzyme using the methods described herein and in co-pending U.S. patent application Ser. Nos. 10/022,073 and 10/022,097, incorporated herein by reference in their entireties), while retaining the ability of these enzymes to activate a prodrug.

Examples of types of enzymes that can be used to make the MDTAs of the present invention include, but are not limited to, proteases, carboxypeptidases, β-lactamases, asparaginases, oxidases, hydrolases, lyases, lipases, cellulases, amylases, aldolases, phosphatases, kinases, tranferases, polymerases, nucleases, nucleotidases, laccases, reductases, and the like. See, e.g., co-pending U.S. patent application Ser. No. 09/954,385, filed Sep. 12, 2001, incorporated herein by reference in its entirety. As such, MDTAs of the invention can, for example, exhibit protease, carboxypeptidase, β-lactamase, asparaginase, oxidase, hydrolase, lyase, lipase, cellulase, amylase, aldolase, phospatase, kinase, tranferase, polymerase, nuclease, nucleotidase, laccase or reductase activity, or the like. Examples of enzymes that can be used are those that can activate a prodrug, discussed below, and those that can produce a toxic agent from a metabolite, e.g., hydrogen peroxide from glucose. See Christofidou-Solomidou et al, 2000, Am J Physiol Lung Cell Mol Physiol 278:L794.

Examples of specific enzymes that can be used to make the MDTAs of the present invention include, but are not limited to, Class A, B, C, or D β-lactamase, β-galactosidase, see Benito et al., FEMS Microbiol. Lett. 123:107 (1994), fibronectin, glucose oxidase, glutathione S-transferase, see Napolitano et al., Chem. Biol. 3:359 (1996) and tissue plasminogen activator, see Smith et al., J. Biol. Chem. 270:30486 (1995).

In one embodiment, an MDTA for use in a human subject comprises an enzyme from a non-human source. In another embodiment, the enzyme is not immunogenic in a human subject.

As described in more detail below, in one aspect the present invention provides a method of treating a subject comprising administering to a subject an MDTA and a prodrug that is a substrate of the MDTA. Enzymes that are useful in this aspect of the invention include, but are not limited to, an alkaline phosphatase useful for converting phosphate-containing prodrugs into free drugs, an arylsulfatase useful for converting sulfate-containing prodrugs into free drugs, a cytosine deaminase useful for converting non-toxic 5-fluorocytosine into the anti-cancer drug 5-fluorouracil, a protease, such as a serine protease, a thermolysin, a subtilisin, a carboxypeptidase and a cathepsin (such as cathepsins B and L), that are useful for converting peptide-containing prodrugs into free drugs, a D-alanylcarboxypeptidase, useful for converting prodrugs that contain D-amino acid substituents, a carbohydrate-cleaving enzyme such as β-galactosidase and a neuraminidase useful for converting glycosylated prodrugs into free drugs, a β-lactamase useful for converting drugs derivatized with β-lactams into free drugs, and a penicillin amidase, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as abzymes, can be used to convert the prodrugs into free active drugs (see, e.g., R. J. Massey, Nature, 328, pp. 457-458 (1987)).

Described in detail below are particular representative, non-limiting classes of enzyme-comprising MDTAs of the invention. Following the teaching provided herein, any other enzyme or enzyme class of interest also can be utilized in a similar fashion to produce an MDTA of the invention.

β-lactamases

In one embodiment, the present invention provides an MDTA comprising β-lactamase (“MDTA-BLA”). In another embodiment, the MDTA-BLA is a targeted enzyme as described in co-pending U.S. patent application Ser. Nos. 10/022,073 and 10/022,097, incorporated herein by reference in their entirety.

In another embodiment, the MDTA-BLA has a specific activity greater than about 0.01 U/pmol against nitrocefin using the assay described in U.S. patent application Ser. No. 10/022,097. In another embodiment, the specific activity is greater than about 0.1 U/pmol. In another embodiment, the specific activity is greater than about 1 U/pmol. Preferably, these specific activities refer to the specific activity of the 4DTA-BLA when it is bound to a microtarget.

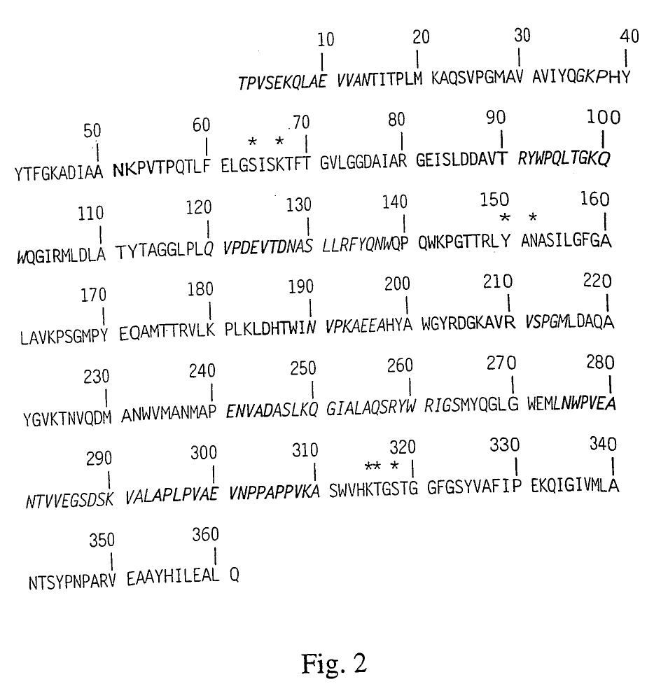

BLA enzymes are widely distributed in both gram-negative and gram-positive bacteria. BLA sequences are well known. A representative example of a BLA sequence is depicted in FIG. 2. BLA enzymes vary in specificity, but have in common that they hydrolyze β-lactams, producing substituted β-amino acids. Thus, they confer resistance to antibiotics containing β-lactams. Because BLA enzymes are not endogenous to mammals, they are subject to minimal interference from inhibitors, enzyme substrates, or endogenous enzyme systems (unlike proteases; see below), and therefore are particularly well-suited for therapeutic administration. BLA enzymes are further well-suited to the therapeutic methods of the present invention because of their small size (BLA from E. cloacae is a monomer of 39 kD; BLA from E. coli is a monomer of 30 kD) and because they have a high specific activity against their substrates and have optimal activity at 37° C. See Melton et al., Enzyme-Prodrug Strategies for Cancer Therapy, Kluwer Academic/Plenum Publishers, New York (1999).

The β-lactamases have been divided into four classes based on their sequences. See Thomson et al., 2000, Microbes and Infection 2:1225-35. The serine β-lactamases are subdivided into three classes: A (penicillinases), C (cephalosporinases) and D (oxacillnases). Class B β-lactamases are the zinc-containing or metallo β-lactamases. Any class of BLA can be utilized to generate an MDTA of the invention.

In one embodiment, the present invention provides a MDTA-BLA that comprises the sequence YXN at its substrate recognition site (throughout, “X” refers to any amino acid residue). In another embodiment, the MDTA-BLA comprises the sequence RLYANASI at its active site. In another embodiment, the MDTA-BLA comprises a sequence at its active site that differs from the sequence RLYANASI by one, two or three amino acid residues. Preferably, the differences are the substitution of conservative amino acid residues. However, insertions, deletions and non-conservative amino acid substitutions also are included.

In another embodiment, the present invention provides a DTA-BLA that comprises the sequence KTXS at its substrate recognition site. In another embodiment, the MDTA-BLA comprises the sequence VHKTGSTG at its active site. In another embodiment, the MDTA-BLA comprises a sequence at its active site that differs from the sequence VHKTGSTG by one, two or three amino acid residues. Preferably, the differences are the substitution of conservative amino acid residues. However, insertions, deletions and non-conservative amino acid substitutions also are included.

In another embodiment, the present invention provides a MDTA-BLA that comprises the sequences YXN and KTXS at its substrate recognition site. In another embodiment, the MDTA-BLA comprises the sequences VHKTGSTG and RLYANASI at its active site. In another embodiment, the MDTA-BLA comprises sequences at its active site that differ from the sequences RLYANASI and VHKTGSTG by one, two or three amino acid residues. Preferably, the differences are the substitution of conservative amino acid residues. However, insertions, deletions and non-conservative amino acid substitutions also are included.

In one embodiment, the BLA enzyme in the MDTA-BLA comprises the amino acid sequence of FIG. 2. In another embodiment, the BLA enzyme in the MDTA-BLA is at least 50%, 60%, 70%, 80%, 90%, 95%, 98% or 99% or more identical to the sequence depicted in FIG. 2.

In another embodiment, a nucleic acid encoding the the BLA enzyme in the MDTA-BLA hybridizes to a nucleic acid complementary to a nucleic acid encoding the amino acid sequence of FIG. 2 under highly stringent conditions. The highly stringent conditions can be, for example, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel et al., eds., 1989, Current Protocols in Moleculat Biology, Vol. I, Green Publishing Associates, Inc., and John Wiley & Sons, Inc., New York, at p. 2.10.3). Other highly stringent conditions can be found in, for example, Current Protocols in Molecular Biology, at pages 2.10.1-16 and Molecular Cloning: A Laboratory Manual, 2d ed., Sambrook et al. (eds.), Cold Spring Harbor Laboratory Press, 1989, pages 9.47-57. In another embodiment, a nucleic acid encoding the BLA enzyme in the MDTA-BLA hybridizes to a nucleic acid complementary to a nucleic acid encoding the amino acid sequence of FIG. 2 under moderately stringent conditions. The moderately stringent conditions can be, for example, washing in 0.2×SSC/0.1% SDS at 42° C. (Ausubel et al., 1989, supra). Other moderately stringent conditions can be found in, for example, Current Protocols in Molecular Biology, Vol. I, Ausubel et al. (eds.), Green Publishing Associates, Inc., and John Wiley & Sons, Inc., 1989, pages 2.10.1-16 and Molecular Cloning: A Laboratory Manual, 2d ed., Sambrook et al. (eds.), Cold Spring Harbor Laboratory Press, 1989, pages 9.47-57.

In another embodiment the invention provides a method of treating a subject by administering to the subject a MDTA-BLA and a prodrug that is converted by the BLA into an active drug. Examples of suitable prodrugs for this embodiment are provided in, e.g., Melton et al., Enzyme-Prodrug Strategies for Cancer Therapy, Kluwer Academic/Plenum Publishers, New York (1999), Bagshaw et al., Current Opinion in Immunology 11:579-83 (1999) and Kerr et al., Bioconjugate Chem. 9:255-59 (1998).

Proteases

In another embodiment, the MDTA comprises a protease. An advantage of proteases is that a peptide can be used as a prodrug. In another embodiment, the protease is human trypsin. Because the enzyme is human, it will not elicit an immune response. It is also smaller than 45,000 Daltons, thus allowing construction of an MDTA-trypsin that is cleared from the circulation by glomular filtration. Optionally, the trypsin is modified so that it does not act on its native substrate. Thus, systemic administration is possible.

It has been reported that a peptide-drug conjugate was specifically cleaved by prostate specific antigen (PSA) at a tumor site. See DeFeo-Jones et al., Nat Med 6:1248 (2000). This report shows the activation of peptide prodrugs at the tumor site is an efficient way to increase the selectivity of an anticancer agent. However, this approach is limited to the treatment of tumors and other diseases where a specific protease is already present in the diseased tissue at concentrations higher than found in other tissues. The present invention allows the addition of exogenous targeted proteases or other enzymes that can recognize and bind to tumor or other target. Consequently, one can decorate the target with a protease or other enzyme that selectively activates a prodrug. This approach allows one to choose an enzyme with suitable kinetic properties instead of relying on the properties of the native endogenous enzyme.

In order to make a MDTA comprising a protease two obstacles should be overcome: the enzyme should not be irreversibly inactivated by compounds in the blood or other relevant tissues, and the enzyme should be selective enough to cause minimal damage to peptides or proteins in the blood or other relevant tissues. In most applications, the MDTA is administered into and subsequently distributed through the circulation to the target tissue. Blood is known to contain numerous protease inhibitors. See Travis & Salvesen, Annu. Rev Biochem 52:655 (1983). Therefore, modified enzymes that remain active in the presence of protease inhibitors located in blood or in the diseased tissue can be used. One important inhibitor in the blood is α2-macroglobulin. This serum protein inhibits proteases, regardless of their mechanisms of action, that are able to cleave the so-called bait region of the inhibitor. For example, see Sottrup-Jensen et al., J Biol Chem 264:15781 (1989). However, there is at least one exception—an extremely selective protease from tobacco etch virus does not cleave α2-macroglobulin and consequently is not inhibited by it. Thus, in one embodiment, an MDTA comprises a catalytic site identical or similar to that of the tobacco etch viral protease. Alternatively, other enzymes with catalytic sites similar to the site of the tobacco etch viral protease can be utilized.

Proteases have been used as therapeutics for acute life-threatening diseases. For example, tissue plasminogen activator (TPA) is a naturally occurring protease that forms a complex with fibrin, the “structural” component of blood clots, that converts plasminogen to plasmin which degrades the fibrin network and dissolves the clot. Since the increase in plasmin concentration occurs acutely and mainly at the clot rather than in the circulation, systemic side effects are reduced. In the case of streptokinase, a bacterial protease administration results in an immunological response which may lead to increased risk of anaphylactic reaction or reduced thrombolytic efficacy on repeat administration.

One embodiment of the present invention relates to a therapeutic MDTA-protease system that: a) evades the circulatory system's protease inhibitors and b) selectively delivers the protease to a target of interest including, e.g., tumor cells, cells infected with a pathogen, or cells undergoing an inflammatory response.

Targeted delivery of a cytotoxic enzyme using an enzyme inhibitor that is released upon entry into the cytosol of a targeted cell or tissue specific cell type bypasses the physiological defense mechanism of protease inhibitors in the blood and allows administration of a useful therapeutic. In one embodiment, this targeting inhibitor, at the same time, binds enzyme to target or has it taken up by the cell. The flexibility of the present therapeutic system can be formatted to be effective at nanomolar doses or less due to the catalytic nature of the released enzyme. Furthermore, this modular approach can be applied to deliver other cytotoxic enzymes that would be detrimental if expressed in blood directly.

In contrast to mammalian proteases, whose small N-terminal zymogen peptides simply prevent premature activation, extracellular bacterial proteases are synthesized with a N-terminal pro region (Pro) that is required for proper folding of the mature protease domain. Because Pro acts as a folding catalyst, a cytotoxic bacterial protease can be selectively delivered to any site of action in the body by first administering a cell specific targeting domain fused to the Pro. After clearance from the blood or other tissues of the Pro-target conjugate, an additional administration of unfolded protease (mature) domain leads to selective folding and activation at the target site. This system overcomes a significant roadblock in the normal application of proteases by administration in human blood since the normal protease inhibitor functions are not activated by the unfolded protease. Furthermore, the enzyme activity can be enhanced by a number of well known techniques that generate sequence diversity leading to altered function and performance profiles such as lowered immunogenicity, increased folding rate, see Wang et al., Biochemistry 37:3165 (1998), or altered substrate specificity. These techniques include, for example, site-directed mutagenesis, random mutagenesis, regiospecific mutagenesis, DNA shuffling techniques, and any combination thereof.

To minimize the hydrolysis of peptides or proteins in the blood or tissues of a patient, the protease of the MDTA can be modified to increase its selectivity towards the prodrug and decrease its selectivity towards endogenous proteins. An example of this embodiment is the use of substrate assisted catalysis described below.

Targets

The targets bound by the MDTA of the present invention can be any substance or composition to which a molecule can be made to bind.

In one aspect, the target is a surface. In one embodiment, the surface is a biological surface. In another embodiment, the biological surface is a surface of an organ. In another embodiment, the biological surface is a surface of a tissue. In another embodiment, the biological surface is a surface of a cell. In another embodiment, the biological surface is a surface of a diseased organ, tissue or cell. In another embodiment, the biological surface is a surface of a normal or healthy organ, tissue or cell. In another embodiment, the surface is a macromolecule in the interstitial space of a tissue. In another embodiment, the biological surface is the surface of a virus or pathogen. In another embodiment, the surface is a non-biological surface. In another embodiment, the non-biological surface is a surface of a medical device. In another embodiment, the medical device is a therapeutic device. In another embodiment, the therapeutic device is an implanted therapeutic device. In another embodiment, the medical device is a diagnostic device. In another embodiment, the diagnostic device is a well or tray.

Sources of cells or tissues include human, animal, bacterial, fungal, viral and plant. Tissues are complex targets and refer to a single cell type, a collection of cell types or an aggregate of cells generally of a particular kind. Tissue may be intact or modified. General classes of tissue in humans include but are not limited to epithelial tissue, connective tissue, nerve tissue, and muscle tissue.

In another aspect, the target is a cancer-related target. The cancer-related target can be any target that a composition of the invention binds to as part of the diagnosis, detection or treatment of a cancer or cancer-associated condition in a subject, for example, a cancerous cell, tissue or organ, a molecule associated with a cancerous cell, tissue or organ, or a molecule, cell, tissue or organ that is associated with a cancerous cell, tissue or organ (e.g., a tumor-bound diagnostic or therapeutic molecule administered to a subject or to a biopsy taken from a subject, or a healthy tissue, such as vasculature, that is associated with cancerous tissue). Examples of cancer-related targets are provided in U.S. Pat. No. 6,261,535, which is incorporated herein by reference in its entirety.

The cancer-related target can be related to any cancer or cancer-associated condition. Examples of types of cancers include carcinomas, sarcomas, myelomas, leukemias, lymphomas and mixed type cancers.

In one embodiment, the cancer is a bone cancer, for example, Ewing's sarcoma, osteosarcoma and rhabdomyosarcoma and other soft-tissue sarcomas. In another embodiment, the cancer is a brain tumor, for example, oligodendroglioma, ependymoma, menengioma, lymplioma, schwannoma or medulloblastoma. In another embodiment, the cancer is breast cancer, for example, ductal carcinoma in situ of the breast. In another embodiment, the cancer is an endocrine system cancer, for example, adrenal, pancreatic, parathyroid, pituitary and thyroid cancers. In another embodiment, the cancer is a gastrointestinal cancer, for example, anal, colorectal, esophogeal, gallbladder, gastric, liver, pancreatic, and small intestine cancers. In another embodiment, the cancer is a gynecological cancer, for example, cervical, endometrial, uterine, fallopian tube, gestational trophoblastic disease, choriocarcinoma, ovarian, vaginal, and vulvar cancers. In another embodiment, the cancer is a head and neck cancer, for example, laryngeal, oropharyngeal, parathryroid or thyroid cancer. In another embodiment, the cancer is a leukemic cancer, for example, acute lymphocytic leukemia, acute myelogenous leukemia, chronic lymphocytic leukemia, chronic myelogenous leukemia, hairy cell leukemia, or a myeloproliferative disorder. In another embodiment, the cancer is a lung cancer, for example, a mesothelioma, non-small cell small cell lung cancer. In another embodiment, the cancer is a lymphoma, for example, AIDS-related lymphoma, cutaneous T cell lymphoma/mucosis fungoides, Hodgkin's disease, or non-Hodgkin's disease. In another embodiment, the cancer is metastatic cancer. In another embodiment, the cancer is a myeloma, for example, a multiple myeloma. In another embodiment, the cancer is a pediatric cancer, for example, a brain tumor, Ewing's sarcoma, leukemia (e.g., acute lymphocytic leukemia or acute myelogenous leukemia), liver cancer, a lymphoma (e.g., Hodgkin's lymphoma or non-Hodgkin's lymphoma), neuroblastoma, retinoblastoma, a sarcoma (e.g., osteosarcoma or rhabdomyosarcoma), or Wilms' Tumor. In another embodiment, the cancer is penile cancer. In another embodiment, the cancer is prostate cancer. In another embodiment, the cancer is a sarcoma, for example, Ewing's sarcoma, osteosarcoma, rhabdomyosarcoma and other soft-tissue sarcomas. In another embodiment, the cancer is a skin cancer, for example, cutaneous T cell lymphoma, mycosis fungoides, Kaposi's sarcoma or melanoma. In another embodiment, the cancer is testicular cancer. In another embodiment, the cancer is thyroid cancer, for example, papillary, follicular, medullary, or anaplastic or undifferentiated thyroid carcinoma. In another embodiment, the cancer is urinary tract cancers, for example, bladder, kidney or urethral cancers. In another embodiment, the cancer or cancer-related condition is ataxia-telangiectasia, carcinoma of unknown primary origin, Li-Fraumeni syndrome, or thymoma.

In another aspect, the cancer-related target is a molecule associated with a cancerous cell or tissue. In one embodiment, the molecule is a tumor or tumor stroma antigen, for example, CD20, CD19, CD30, CD3, GD2, Lewis-Y, 72 kd glycoprotein (gp72, decay-accelerating factor, CD55, DAF, C3/C5 convertases), CO17-1A (EpCAM, 17-1A, EGP-40), TAG-72, CSAg-P (CSAp), 45 kd glycoprotein, HT-29 ag, NG2, A33 (431 kd gp), 38 kd gp, MUC-1, CEA, EGFR (HER1), HER2, HER3, HER4, HN-1 ligand, CA125, syndecan-1, Lewis X, PgP, FAP stromal Ag (fibroblast activation protein), EDG receptors (endoglin receptors), ED-B, laminin-5 (gamma2), cox-2 (+LN-5), PgP (P-glycoprotein), alphaVbeta3 integrin, alphaVbeta5, integrin, uPAR (urokinase plasminogen activator receptor), endoglin (CD105), folate receptor osteopontin (EDG 1,3), p97 (melanotransferrin), farnesyl transferase or a molecule in an apoptotic pathway (e.g., a death receptor, fas, caspase or bcl-2) or a lectin.

In another aspect, the target is a hematopoietic cell. Hematopoietic cells encompass hematopoietic stem cells (HSCs), erythrocytes, neutrophils, monocytes, platelets, mast cells, eosinophils, basophils, B and T cells, macrophages, and natural killer cells. In one embodiment, the HSC has a surface antigen expression profile of CD34⁺Thy-1⁺, and preferably CD34⁺Thy-1⁺Lin⁻. Lin⁻ refers to a cell population selected on the basis of the lack of expression of at least one lineage specific marker. Methods for isolating and selecting HSCs are well known in the art and reference is made to U.S. Pat. Nos. 5,061,620, 5,677,136, and 5,750,397, each of which is incorporated herein in its entirety.

In another aspect, the target is a molecule. In one embodiment, the molecule is an organic molecule. In another embodiment, the molecule is a biological molecule. In another embodiment, the biological molecule is a cell-associated molecule. In another embodiment, the cell-associated molecule is associated with the outer surface of a cell. In another embodiment, the cell-associated molecule is part of the extracellular matrix. In another embodiment, the cell-associated molecule is associated with the outer surface of a cell is a protein. In another embodiment, the protein is a receptor. In another embodiment, the cell-associated molecule is specific to a type of cell in a subject. In another embodiment, the type of cell is a diseased cell. In another embodiment, the diseased cell is a cancer cell. In another embodiment, the diseased cell is an infected cell. Other molecules that can serve as targets according to the invention include, but are not limited to, proteins, peptides, nucleic acids, carbohydrates, lipids, polysaccharides, glycoproteins, hormones, receptors, antigens, antibodies, toxic substances, metabolites, inhibitors, drugs, dyes, nutrients and growth factors.

Non-limiting examples of protein and chemical targets encompassed by the invention include chemokines and cytokines and their receptors. Cytokines as used herein refer to any one of the numerous factors that exert a variety of effects on cells, for example inducing growth or proliferation. Non-limiting examples include interleukins (IL), IL-2, IL-3, IL-4 IL-6, IL-10, IL-12, IL-13, IL-14 and IL-16; soluble IL-2 receptor; soluble IL-6 receptor; erythropoietin (EPO); thrombopoietin (TPO); granulocyte macrophage colony stimulating factor (GM-CSF); stem cell factor (SCF); leukemia inhibitory factor (LIF); interferons; oncostatin M (OM); the immunoglobulin superfamily; tumor necrosis factor (TNF) family, particularly TNF-α; TGFβ; and IL-1α; and vascular endothelial growth factor (VEGF) family, particularly VEGF (also referred to in the art as VEGF-A), VEGF-B, VEGF-C, VEGF-D and placental growth factor (PLGF). Cytokines are commercially available from several vendors including Amgen (Thousand Oaks, Calif.), Immunex (Seattle, Wash.) and Genentech (South San Francisco, Calif.). Particularly preferred are VEGF and TNF-α. Antibodies against TNF-α show that blocking interaction of the TNF-α with its receptor is useful in modulating over-expression of TNF-α in several disease states such as septic shock, rheumatoid arthritis, or other inflammatory processes. VEGF is an angiogenic inducer, a mediator of vascular permeability, and an endothelial cell specific mitogen. VEGF has also been implicated in tumors. Targeting members of the VEGF family and their receptors may have significant therapeutic applications, for example blocking VEGF may have therapeutic value in ovarian hyper stimulation syndrome (OHSS). Reference is made to N. Ferrara et al. (1999) Nat. Med. 5:1359 and Gerber et al., (1999) Nat. Med. 5:623. Other preferred targets include cell-surface receptors, such as T-cell receptors.

Chemokines are a family of small proteins that play an important role in cell trafficking and inflammation. Members of the chemokine family include, but are not limited to, IL-8, stomal-derived factor-1(SDF-1), platelet factor 4, neutrophil activating protein-2 (NAP-2) and monocle chemo attractant protein-1 (MCP-1).

Other protein and chemical targets include, but are not limited to: immunoregulation modulating proteins, such as soluble human leukocyte antigen (HLA, class I and/or class II, and non-classical class I HLA (E, F and G)); surface proteins, such as soluble T or B cell surface proteins, human serum albumin; arachadonic acid metabolites, such as prostaglandins, leukotrienes, thromboxane and prostacyclin; IgE, auto or alloantibodies for autoimmunity or allo- or xenoimmunity. Ig Fc receptors or Fc receptor binding factors; G-protein coupled receptors; cell-surface carbohydrates; angiogenesis factors; adhesion molecules; ions, such as calcium, potassium, magnesium, aluminum, and iron; fibril proteins, such as prions and tubulin; enzymes, such as proteases, aminopeptidases, kinases, phosphatases, DNAses, RNAases, lipases, esterases, dehydrogenases, oxidases, hydrolases, sulphatases, cyclases, transferases, transaminases, carboxylases, decarboxylases, superoxide dismutase, and their natural substrates or analogs; hormones and their corresponding receptors, such as follicle stimulating hormone (FSH), leutinizing hormone (LH), thyroxine (T4 and T3), apolipoproteins, low density lipoprotein (LDL), very low density lipoprotein (VLDL), cortisol, aldosterone, estriol, estradiol, progesterone, testosterone, dehydroepiandrosterone (DHBA) and its sulfate (DHEA-S); peptide hormones, such as renin, insulin, calcitonin, parathyroid hormone (PTH), human growth hormone (hGH), vasopressin and antidiuretic hormone (AD), prolactin, adrenocorticotropic hormone (ACTH), LHRH, thyrotropin-releasing hormone (THRH), vasoactive intestinal peptide (JIP), bradykinin and corresponding prohormones; catechcolamines such as adrenaline and metabolites; cofactors including atrionatriutic factor (AdF), vitamins A, B, C, D, E and K, and serotonin; coagulation factors, such as prothrombin, thrombin, fibrin, fibrinogen, Factor VIII, Factor IX, Factor XI, and von Willebrand factor; plasminogen factors, such as plasmin, complement activation factors, LDL and ligands thereof, and uric acid; compounds regulating coagulation, such as hirudin, hirulog, hementin, hepurin, and tissue plasminigen activator (TPA); nucleic acids for gene therapy; compounds which are enzyme antagonists; and compounds binding ligands, such as inflammation factors; and receptors and other proteins that bind to one or more of the preceding molecules.

Non-human derived targets include without limitation drugs, especially drugs subject to abuse, such as cannabis, heroin and other opiates, phencyclidine (PCP), barbiturates, cocaine and its derivatives, and benzadiazepine; toxins, such as heavy metals like mercury and lead, arsenic, and radioactive compounds; chemotherapeutic agents, such as paracetamol, digoxin, and free radicals; bacterial toxins, such as lipopolysaccharides (LPS) and other gram negative toxins, Staphylococcus toxins, Toxin A, Tetanus toxins, Diphtheria toxin and Pertussis toxins; plant and marine toxins; snake and other venoms, virulence factors, such as aerobactins, or pathogenic microbes; infectious viruses, such as hepatitis, cytomegalovirus (CMV), herpes simplex virus (HSV types 1, 2 and 6), Epstein-Barr virus (EBV), varicella zoster virus (VZV), human immunodeficiency virus (HIV-1, -2) and other retroviruses, adenovirus, rotavirus, influenzae, rhinovirus, parvovirus, rubella, measles, polio, pararyxovirus, papovavirus, poxvirus and picomavirus, prions, plasmodia tissue factor, protozoans, such as Entamoeba histolitica, Filaria, Giardia, Kalaazar, and toxoplasma; bacteria, gram-negative bacteria responsible for sepsis and nosocomial infections such as E. coli, Acynetobacter, Pseudomonas, Proteus and Klebsiella, also gram-positive bacteria such as Staphylococcus, Streptococcus, Meningococcus and Llycobacteria, Chlamydiae Legionnella and Anaerobes; fungi such as Candida, Pneumocystis, Aspergillus, and Mycoplasma.

In one aspect the target includes an enzyme such as proteases, aminopeptidases, kinases, phosphatases, DNAses, RNAases, lipases, esterases, dehydrogenases, oxidases, hydrolases, sulphatases, cellulases, cyclases, transferases, transaminases, carboxylases, decarboxylases, superoxide dismutase, and their natural substrates or analogs. Particularly preferred enzymes include hydrolases, particularly alpha/beta hydrolases; serine proteases, such as subtilisins, and chymotrypsin serine proteases; cellulases; and lipases.

In another embodiment, the target is a non-biological material. In another embodiment, the non-biological material is a fabric. In another embodiment, the fabric is a natural fabric. In another embodiment, the fabric is cotton. In another embodiment, the fabric is silk. In another embodiment, the fabric is wool. In another embodiment, the fabric is a non-natural fabric. In another embodiment, the fabric is nylon. In another embodiment, the fabric is rayon. In another embodiment, the fabric is polyester. In another embodiment, the non-biological material is a plastic. In another embodiment, the non-biological material is a ceramic. In another embodiment, the non-biological material is a metal. In another embodiment, the non-biological material is rubber. In another embodiment, the non-biological material is wood.

In another embodiment the target is a microcircuit. This circuit can be in its finished form or in any stage of circuit manufacturing. Binding of the MDTA to the microcircuit can be dependent on any milieu condition, for example, light. The MDTA can be used to remove or deposit a compound onto the circuit, for example, an n-type dopant (e.g., arsenic, phosphorus, antimony, titanium or other donor atom species) or a p-type dopant (e.g., boron, aluminum, gallium, indium or other acceptor atom species). See, e.g., van Zant, 2000, Microchip Fabrication, McGraw-Hill, New York, incorporated herein by reference in its entirety.

In another embodiment, the target is not an antibody (e.g., a polyclonal antibody, a monoclonal antibody, an scFv, or another antigen-binding fragment of an antibody).

Microtargets

The microtarget is the portion or portions of the target bound by the binding moiety. The microtarget can comprise any kind of molecule, or a portion of a molecule, or a plurality of molecules or portions of molecules, for example, all or part of any of the targets discussed above. The microtarget can be known or unknown to the operator. Examples of types of microtargets include peptides, polypeptides or proteins (e.g., antibodies, antibody fragments (for example, single chain antibody variable region fragment (scFv), ligand-binding peptides, polypeptides or proteins, receptor-binding peptides, polypeptides or proteins or an epitope), organic molecules (e.g., sugars, lipids, amino acids, nucleotides or small organic molecules) or inorganic molecules. In one embodiment, the microtarget is associated with a cell, for example, a cell surface marker. In a more particularly defined embodiment, the microtarget associated with a cell is a tumor antigen (e.g., a carcinoembryonic antigen, p97, A33, or MUC-1).

Milieu and Reaction Conditions

A milieu is the molecular environment in which a microtarget encounters an MDTA, and can be characterized by the presence, concentration, magnitude or amount of, for example, a solvent (e.g., an aqueous solvent), temperature, light, one or more ions or molecules (e.g., metals, gases, salts, sugars, amino acids, nucleotides, peptides, polypeptides, proteins, nucleic acids, oligonucleotides, polynucleotides or metabolites), or anything else that can affect the binding of one molecule to another.

Any aspect of the reaction conditions can affect the binding affinity of the MDTA for its target. The aspect of the reaction conditions that affects binding affinity of the MDTA for its target need not be known. Examples of aspects of the reaction conditions that can affect binding affinity of the MDTA for its target include, but are not limited to, pH, partial pressure of a gas (e.g., O₂ or CO₂), concentration of a solute (e.g., lactic acid, a sugar, or another organic or inorganic molecule), temperature, light or ionic strength.

In one embodiment, the target is a cancer cell or a tumor comprising a microtarget in a milieu that allows an MDTA to bind to the microtarget better than the MDTA binds to the microtarget on a target having a different milieu. Any difference between a cancer cell or tumor and another cell or tissue, e.g., a healthy cell or tissue, can be exploited to make an MDTA that preferentially binds a microtarget in the cancer cell or tumor over the microtarget in the other cell or tissue. It has been well documented that most tumor tissues, regardless of the type of cancer involved, have a lower pH in their interstitial compartments as compared to most healthy tissues. See Griffiths et al., 2001, Novartis Found Symp. 240: 46-62. Thus, in one embodiment, the MDTA binds a microtarget present on a cancer cell or tumor at a lower pH but not at a higher pH. The low pH in tumors can be further enhanced by a variety of treatments, for example, elevated glucose levels or the administration of mitochondrial inhibitors and others. See Kuin et al., 1999, Br J Cancer 79:793-801, Evelhoch, 2001 Novartis Found Symp 240: 68-84. In one embodiment, an MDTA for targeting a cancer cell or tissue comprises a pH sensitive binding moiety and an active moiety that is more active at a lower pH than at a higher pH. See, e.g., Bellnier et al., 1999, Photochem Photobiollo, 70: 630-36; Arano et al., 1994, J Nucl Med 35, 326-33; Boyer et al., 1993, Br J Cancer 67:81-87; Prokof'eva et al., 1990, Izv Akad Nauk SSSR Biol 338-42; Jensen, 1994, Cancer Res 54:2959-63; Amtmann, 2001, Cancer Chemoother Pharmacol 47, 461-66; Adams et al., 2000, Cancer Chemother Pharmacol 46:263-71.

Method of Making MDTAs

In another aspect the invention provides methods of making MDTAs. Any method for making a binding moiety that preferentially binds a microtarget in a first milieu over binding of the microtarget in a second milieu can be used.

In one embodiment, a library of variants of a peptide sequence is tested for milieu-dependent binding. Any peptide sequence can be used. In another embodiment, the peptide sequence is one that binds to a microtarget, and variants are identified that bind to the microtarget in a milieu-dependent fasion. Of particular interest is the derivatization of residues in a targeted molecule that are known to contact the microtarget. For instance, in an antibody or scFv one would mutate the CDRs as mutations in these regions are particularly likely to yield milieu-dependent variants. In another embodiment, the peptide sequence binds to the microtarget in a milieu-dependent fashion, and variants of the peptide sequence are screened to identify variants that show increased milieu-dependent binding to the microtarget. In another embodiment, an iterative process is used whereby a variant identified as exhibiting milieu-dependent binding to a microtarget in a previous round is the peptide sequence that is derivatized to generate the library of the subsequent round.

A number of methods have been described that allow one to enrich and identify molecules with desired binding properties from a large library of variants. Examples of such methods are phage display, ribosomal display and cell display. These methods can be adapted to enrich mutants showing milieu-dependent binding.

In another embodiment, a library of variants can be contacted with a microtarget under a first set of conditions. Variants that show weak or no interaction can be removed. The remaining variants can be identified using mass spectrometry. The process can be repeated with the same library under a second set of conditions. Comparison of the amino acid sequence and abundance of variants obtained by both processes will reveal variants that bind to the target in a milieu-dependent way.

In another embodiment, a strategy is employed to generate a library with a large fraction of milieu-dependent binding molecules. For example, one can generate a library of variants of a peptide that binds to a microtarget by focusing the mutagenesis on positions in the peptide known to contact the microtarget, e.g., by completely randomizing one or more such positions by site saturation mutagenesis using the random codon NNS wherein N is a mixture of G, C, T and A nucleotides and S is a mixture of G and C nucleotides. See Olins et al., 1995, J Biol Chem 270:23754-60. In another embodiment, one can use a randomization scheme that leads to the introduction of charged amino acid residues. For example, use of the random codon VAC, wherein V is a mixture of C, A and G nucleotides and M is a mixture of C and A nucleotides, leads to the introduction of histidine, asparagine and aspartate residues. Use of random codon SAT, where S=G or C nucleotides, leads to the introduction of a histidine or asparagines. Use of the random codon VAM, wherein V is as defined above and M is a mixture of C and A nucleotides, leads to the introduction of histidine, asparagine, aspartate, glutamine, lysine, and glutamine. Other randomization schemes can be used as well. In another embodiment, site directed mutations are introduced into the region of the binding moiety that is in contact with or in proximity to the microtarget, for example, mutations that introduce one or more charged residues (e.g., histidine, aspartate, glutamate, lysine or arginine). In another embodiment, one or more surfaced-exposed residues of the MDTA are replaced with other amino acids, e.g., with charged amino acids such as histidine, aspartate, glutamate, lysine or arginine.

Any method of identifying or detecting a MDTA or candidate MDTA bound to a target can be used. For example, the MDTA can be detectably labeled, e.g., with a labeling moiety that is radioactive, light-emitting, flourescent, or with a moiety that has a detectable activity, e.g., a detectable enzymatic activity. The moiety used to detect bound MDTA can be non-covalently bound to the MDTA, e.g., using an anti-MDTA antibody that is detectably labeled, as in an ELISA reaction, covalently bound to the MDTA, e.g. directly covalently linked to the MDTA, or through one or more covalent linking molecules. Alternatively, an affinity maturation approach can be used, e.g., as discussed in copending U.S. Pat. App. Ser. No. 60/388,387 (attorney docket no. 9342-0040-999), filed concurrently with the present application, incorporated herein by reference in its entirety. Other methods of detecting an MDTA bound to a target are provided in copending U.S. Pat. App. Ser. No. 60/279,609 (attorney docket no. 9342-041-999) and U.S. Ser. No. 10/170,387 (attorney docket no. 9342-043-999), filed concurrently with the present application, incorporated herein by reference in their entireties. Alternatively, phage display may be used, as shown, for example, in U.S. Pat. No. 5,837,500, incorporated herein by reference in their entireties.

Alternatively, MDTAs can be isolated from random libraries of prototype targeted agents by phage display or similar method that links a binding moiety to an identifiable tag (see, for example U.S. patent application No. 5,837,500, incorporated by reference in its entirety, including any drawings. One can contact the library with the microtarget under one set of conditions and then elute bound molecules by changing the milieu (e.g., one can elute under acidic conditions). By this process, MDTAs can be enriched from the random population of prototype variants.

Nucleic Acids and Methods of Making MDTAs

In another aspect, the present invention provides a nucleic acid encoding a polypeptide comprising all or part of an MDTA. The nucleic acid can be, for example, a DNA or an RNA. The present invention also provides a plasmid comprising a nucleic acid encoding a polypeptide comprising all or part of an MDTA. The plasmid can be, for example, an expression plasmid that allows expression of the polypeptide in a host cell or organism, or ill vitro. The expression vector can allow expression of the polypeptide in, for example, a bacterial cell. The bacterial cell can be, for example, an E. coli cell.

Because of the redundancy in the genetic code, typically a large number of DNA sequences encode any given amino acid sequence and are, in this sense, equivalent. As described below, it may be desirable to select one or another equivalent DNA sequences for use in a expression vector, based on the preferred codon usage of the host cell into which the expression vector will be inserted. The present invention is intended to encompass all DNA sequences that encode the MDTA.

Production of the MDTA of the invention can be carried out using a recombinant expression clone. The construction of the recombinant expression clone, the transformation of a host cell with the expression clone, and the culture of the transformed host cell under conditions which promote expression, can be carried out in a variety of ways using techniques of molecular biology well understood in the art. Methods for each of these steps are described in general below. Preferred methods are described in detail in the examples.

An operable expression clone is constructed by placing the coding sequence in operable linkage with a suitable control sequences in an expression vector. The vector can be designed to replicate autonomously in the host cell or to integrate into the chromosomal DNA of the host cell. The resulting clone is used to transform a suitable host, and the transformed host is cultured under conditions suitable for expression of the coding sequence. The expressed MDTA is isolated from the medium or from the cells, although recovery and purification of the MDTA may not be necessary in some instances.

Construction of suitable clones containing the coding sequence and a suitable control sequence employs standard ligation and restriction techniques that are well understood in the art. In general, isolated plasmids, DNA sequences, or synthesized oligonucleotides are cleaved, modified, and religated in the form desired. Suitable restriction sites can, if not normally available, be added to the ends of the coding sequence so as to facilitate construction of an expression clone.

Site-specific DNA cleavage is performed by treating with a suitable restriction enzyme (or enzymes) under conditions that are generally understood in the art and specified by the manufacturers of commercially available restriction enzymes. See, e.g., product catalogs from Amersham (Arlington Heights, Ill.), Roche Molecular Biochemicals (Indianapolis, Ind.), and New England Biolabs (Beverly, Mass.). In general, about 1 μg of plasmid or other DNA is cleaved by one unit of enzyme in about 20 μl of buffer solution; in the examples below, an excess of restriction enzyme is generally used to ensure complete digestion of the DNA. Incubation times of about one to two hours at a temperature which is optimal for the particular enzyme are typical. After each incubation, protein is removed by extraction with phenol and chloroform; this extraction can be followed by ether extraction and recovery of the DNA from aqueous fractions by precipitation with ethanol. If desired, size separation of the cleaved fragments may be performed by polyacrylamide gel or agarose gel electrophoresis using standard techniques. See, e.g., Maxam et al., 1980, Methods in Enzymology 65:499-560.

Restriction enzyme-cleaved DNA fragments with single-strand “overhanging” termini can be made blunt-ended (double-strand ends) by, for example, treating with the large fragment of E. coli_DNA polymerase I (Klenow) in the presence of the four deoxynucleoside triphosphates (dNTPs) using incubation times of about 15 to 25 minutes at 20° C. to 25° C. in 50 mM Tris, pH 7.6, 50 mM NaCl, 10 mM MgCl₂, 10 mM DTT, and 5 to 10 μM dNTPs. The Klenow fragment fills in at 5′ protruding ends, but chews back protruding 3′ single strands, even though the four dNTPs are present. If desired, selective repair can be performed by supplying one or more selected dNTPs, within the limitations dictated by the nature of the protruding ends. After treatment with Klenow, the mixture is extracted with phenol/chloroform and ethanol precipitated. Similar results can be achieved using S1 nuclease, because treatment under appropriate conditions with S1 nuclease results in hydrolysis of any single-stranded portion of a nucleic acid.

Ligations can be performed, for example, in 15-30 μl volumes under the following standard conditions and temperatures: 20 mM Tris-Cl, pH 7.5, 10 mM MgCl₂; 10 mM DTT, 33 μg/ml BSA 10-50 mM NaCl, and either 40 μM ATP and 0.01-0.02 (Weiss) units T4 DNA ligase at 0° C. (for ligation of fragments with complementary single-stranded ends) or 1 mM ATP and 0.3-0.6 units T4 DNA ligase at 14° C. (for “blunt end” ligation). Intermolecular ligations of fragments with complementary ends are usually performed at 33-100 μg/ml total DNA concentrations (5-100 nM total ends concentration). Intermolecular blunt end ligations (usually employing a 20-30 fold molar excess of linkers, optionally) are performed at 1 μM total ends concentration.

In vector construction, the vector fragment is commonly treated with bacterial or calf intestinal alkaline phosphatase (BAP or CIAP) to remove the 5′ phosphate and prevent religation and reconstruction of the vector. BAP and CIAP digestion conditions are well known in the art, and published protocols usually accompany the commercially available BAP and CIAP enzymes. To recover the nucleic acid fragments, the preparation is extracted with phenol-chloroform and ethanol precipitated to remove the phosphatase and purify the DNA. Alternatively, religation of unwanted vector fragments can be prevented by restriction enzyme digestion before or after ligation, if appropriate restriction sites are available.

Correct ligations for plasmid construction can be confirmed using any suitable method known in the art. For example, correct ligations for plasmid construction can be confirmed by first transforming a suitable host, such as E. coli strain DG101 (ATCC 47043) or E. coli strain DG116 (ATCC 53606), with the ligation mixture. Successful transformants are selected by ampicillin, tetracycline or other antibiotic resistance or sensitivity or by using other markers, depending on the mode of plasmid construction, as is understood in the art. Plasmids from the transformants are then prepared according to the method of Clewell et al., 1969, Proc. Natl. Acad. Sci. USA 62:1159, optionally following chloramphenicol amplification. See Clewell, 1972, J. Bacteriol. 110:667. Alternatively, plasmid DNA can be prepared using the “Base-Acid” extraction method at page 11 of the Bethesda Research Laboratories publication Focus 5 (2), and very pure plasmid DNA can be obtained by replacing steps 12 through 17 of the protocol with CsCl/ethidium bromide ultracentrifugation of the DNA. As another alternative, a commercially available plasmid DNA isolation kit, e.g., HISPEED™, QIAFILTER™ and QIAGEN® plasmid DNA isolation kits (Qiagen, Valencia Calif.) can be employed following the protocols supplied by the vendor. The isolated DNA can be analyzed by, for example, restriction enzyme digestion and/or sequenced by the dideoxy method of Sanger et al., 1977, Proc. Natl. Acad. Sci. USA 74:5463, as further described by Messing et al., 1981, Nuc. Acids Res. 9:309, or by the method of Maxam et al., 1980, Methods in Enzymology 65:499.

The control sequences, expression vectors, and transformation methods are dependent on the type of host cell used to express the gene. Generally, procaryotic, yeast, insect, or mammalian cells are used as hosts. Procaryotic hosts are in general the most efficient and convenient for the production of recombinant proteins and are therefore preferred for the expression of the protein.

The procaryote most frequently used to express recombinant proteins is E. coli. However, microbial strains other than E. coli can also be used, such as bacilli, for example Bacillus subtilis, various species of Pseudomonas and Salmonella, and other bacterial strains. In such procaryotic systems, plasmid vectors that contain replication sites and control sequences derived from the host or a species compatible with the host are typically used.

For expression of constructions under control of most bacterial promoters, E. coli K12 strain MM4294. obtained from the E. coli Genetic Stock Center under GCSC #6135, can be used as the host. For expression vectors with the P_{L}N_{RBS} or P_{L} T⁷_{RBS} control sequence, E. coli K12 strain MC1000 lambda lysogen, N₇N₅₃cI857 SusP₈₀, ATCC 39531, may be used. E. coli DG116. which was deposited with the ATCC (ATCC 53606) on Apr. 7, 1987, and E. coli KB2, which was deposited with the ATCC (ATCC 53075) on Mar. 29, 1985, are also useful host cells. For M13 phage recombinants, E. coli strains susceptible to phage infection, such as E. coli K12 strain DG98 (ATCC 39768), are employed. The DG98 strain was deposited with the ATCC on Jul. 13, 1984.

For example, E. coli is typically transformed using derivatives of pBR322, described by Bolivar et al., 1977, Gene 2:95. Plasmid pBR322 contains genes for ampicillin and tetracycline resistance. These drug resistance markers can be either retained or destroyed in constructine the desired vector and so help to detect the presence of a desired recombinant. Commonly used prokaryotic control sequences, i.e., a promoter for transcription initiation, optionally with an operator, along with a ribosome binding site sequence, include the β-lactamase (penicillinase) and lactose (lac) promoter systems, see Chang et al., 1977, Nature 198:1056, the tryptophan (trp) promoter system, see Goeddel et al., 1980, Nuc. Acids Res. 8:4057, and the lambda-derived P_{L} promoter, see Shimatake et al., 1981, Nature 292:128, and gene N ribosome binding site (N_{RBS}). A portable control system cassette is set forth in U.S. Pat. No. 4,711,845, issued Dec. 8, 1987. This cassette comprises a P_{L} promoter operably linked to the N_{RBS} in turn positioned upstream of a third DNA sequence having at least one restriction site that permits cleavage within six base pairs 3′ of the N_{RBS} sequence. Also useful is the phosphatase A (phoA) system described by Chang et al., in European Patent Publication No. 196,864, published Oct. 8, 1986. However, any available promoter system compatible with procaryotes can be used to construct a expression vector of the invention.

In addition to bacteria, eucaryotic microbes, such as yeast, can also be used as recombinant host cells. Laboratory strains of Saccharomyces cerevisiae, Baker's yeast, are most often used, although a number of other strains are commonly available. While vectors employing the two micron origin of replication are common, see Broach, 1983, Meth. Enz. 101:307, other plasmid vectors suitable for yeast expression are known. See, e.g., Stinchcomb et al., 1979, Nature 282:39; Tschempe et al., 1980, Gene 10:157; and Clarke et al., 1983, Meth. Enz. 101:300. Control sequences for yeast vectors include promoters for the synthesis of glycolytic enzymes. See Hess et al., 1968, J. Adv. Enzyme Reg. 7:149; Holland et al., 1978, Biotechnology 17:4900; and Holland et al., 1981, J. Biol. Chem. 256:1385. Additional promoters known in the art include the promoter for 3-phosphoglycerate kinase, see Hitzeman et al., 1980, J. Biol. Chem. 255:2073, and those for other glycolytic enzymes, such as glyceraldehyde 3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other. promoters that have the additional advantage of transcription controlled by growth conditions are the promoter regions for alcohol dehydrogenase 2, isocytochrome C, acid phosphatase, degradative enzymes associated with nitrogen metabolism, and enzymes responsible for maltose and galactose utilization (Holland, supra).

Terminator sequences may also be used to enhance expression when placed at the 3′ end of the coding sequence. Such terminators are found in the 3′ untranslated region following the coding sequences in yeast-derived genes. Any vector containing a yeast-compatible promoter, origin of replication, and other control sequences is suitable for use in constructing yeast expression vectors.

The coding sequence can also be expressed in eucaryotic host cell cultures derived from multicellular organisms. See, e.g., Tissue Culture, Academic Press, Cruz and Patterson, editors (1973). Useful host cell lines include COS-7, COS-A2, CV-1, murine cells such as murine myelomas N51 and VERO, HeLa cells, and Chinese hamster ovary (CHO) cells. Expression vectors for such cells ordinarily include promoters and control sequences compatible with mammalian cells such as, for example, the commonly used early and late promoters from Simian Virus 40 (SV 40), see Fiers et al., 1978, Nature 273:113, or other viral promoters such as those derived from polyoma, adenovirus 2, bovine papilloma virus (BPV), or avian sarcoma viruses, or immunoglobulin promoters and heat shock promoters. A system for expressing DNA in mammalian systems using a BPV vector system is disclosed in U.S. Pat. No. 4,419,446. A modification of this system is described in U.S. Pat. No. 4,601,978. General aspects of mammalian cell host system transformations have been described by Axel, U.S. Pat. No. 4,399,216. “Enhancer” regions are also important in optimizing expression; these are, generally, sequences found upstream of the promoter region. Origins of replication may be obtained, if needed, from viral sources. However, integration into the chromosome is a common mechanism for DNA replication in eucaryotes.

Plant cells can also be used as hosts, and control sequences compatible with plant cells, such as the nopaline synthase promoter and polyadenylation signal sequences, see Depicker et al., 1982, J. Mol. Appl. Gen. 1:561, are available. Expression systems employing insect cells utilizing the control systems provided by baculovirus vectors have also been described. See Miller et al., in Genetic Engineering (1986), Setlow et al., eds., Plenum Publishing, Vol. 8, pp. 277-97. Insect cell-based expression can be accomplished in Spodoptera frugipeida. These systems are also successful in producing recombinant enzymes.

Depending on the host cell used, transformation is done using standard techniques appropriate to such cells. The calcium treatment employing calcium chloride, as described by Cohen, 1972, Proc. Natl. Acad. Sci. USA 69:2110 is used for procaryotes or other cells that contain substantial cell wall barriers. Infection with Agrobacterium tumefaciens, see Shaw et al., 1983, Gene 23:315, is used for certain plant cells. For mammalian cells, the calcium phosphate precipitation method of Graham et al., 1978, Virology 52:546 is preferred. Transformations into yeast are carried out according to the method of Van Solingen et al., 1977, J. Bact. 130:946, and Hsiao et al., 1979, Proc. Natl. Acad. Sci. USA 76:3829.

It may be desirable to modify the sequence of a DNA encoding a polypeptide comprising all or part of an MDTA of the invention to provide, for example, a sequence more compatible with the codon usage of the host cell without modifying the amino acid sequence of the encoded protein. Such modifications to the initial 5-6 codons may improve expression efficiency. DNA sequences which have been modified to improve expression efficiency, but which encode the same amino acid sequence, are considered to be equivalent and encompassed by the present invention.

A variety of site-specific primer-directed mutagenesis methods are available and well-known in the art. See, e.g., Sambrook et al., Molecular Cloning: A Laboratory Manual, Cold Spring Harbor, 1989, second edition, chapter 15.51, “Oligonucleotide-mediated mutagenesis,” which is incorporated herein by reference. The polymerase chain reaction (PCR) can be used to perform site-specific mutagenesis. In another technique now standard in the art, a synthetic oligonucleotide encoding the desired mutation is used as a primer to direct synthesis of a complementary nucleic acid sequence contained in a single-stranded vector, such as pBSM13+ derivatives, that serves as a template for construction of the extension product of the mutagenizing primer. The mutagenized DNA is transformed into a host bacterium, and cultures of the transformed bacteria are plated and identified. The identification of modified vectors may involve transfer of the DNA of selected transformants to a nitrocellulose filter or other membrane and the “lifts” hybridized with kinased synthetic mutagenic primer at a temperature that permits hybridization of an exact match to the modified sequence but prevents hybridization with the original unmutagenized strand. Transformants that contain DNA that hybridizes with the probe are then cultured (the sequence of the DNA is generally confirmed by sequence analysis) and serve as a reservoir of the modified DNA.

Once the polypeptide has been expressed in a recombinant host cell, purification of the polypeptide may be desired. A variety of purification procedures can be used.

For long-term stability, the purified polypeptide can be stored in a buffer that contains one or more non-ionic polymeric detergents. Such detergents are generally those that have a molecular weight in the range of approximately 100 to 250,00 preferably about 4,000 to 200,000 daltons and stabilize the enzyme at a pH of from about 3.5 to about 9.5, preferably from about 4 to 8.5. Examples of such detergents include those specified on pages 295-298 of McCutcheon's Emulsifiers & Detergents, North American edition (1983), published by the McCutcheon Division of MC Publishing Co., 175 Rock Road, Glen Rock, N.J. (USA), the entire disclosure of which is incorporated herein by reference. Preferably, the detergents are selected from the group comprising ethoxylated fatty alcohol ethers and lauryl ethers, ethoxylated alkyl phenols, octylphenoxy polyethoxy ethanol compounds, modified oxyethylated and or oxypropylated straight-chain alcohols, polyethylene glycol monooleate compounds, polysorbate compounds, and phenolic fatty alcohol ethers. More particularly preferred are Tween 20™, a polyoxyethylated (20) sorbitan monolaurate from ICI Americas Inc. (Wilmington, Del.). and Iconol™ NP-40; an ethoxylated alkyl phenol (nonyl) from BASF Wyandotte Corp. (Parsippany, N.J.).

MDTA Prodrug Therapy

In one aspect the present invention provides a method of treating a subject by administering a MDTA and a prodrug, wherein the MDTA is specifically localized to a portion of the subject's body where it converts the prodrug into an active drug. In one embodiment, the MDTA is an ADEPT molecule, see. e.g., Xu et al., 2001, Clin. Cancer Res. 11:3314-24; Denny, 2001, Eur. J. Med. Chem 36:577-95, or a targeted enzyme, for example, as described in U.S. patent application Ser. Nos. 10/022,073 and 10/022,097, incorporated herein by reference in their entireties.

The prodrug therapy methods of the present invention provide advantages over previously available methods. Previously available targeted delivery approaches require an intrinsic overexpression of a microtarget on a target tissue (e.g., tumor tissue) compared to a non-target tissue (e.g., normal cells), yet most target antigens are present in significant amounts on non-target tissues. The MDTAs of the invention can preferentially bind to a microtarget on a target tissue, even if it is found in significant amounts on a non-target tissue.

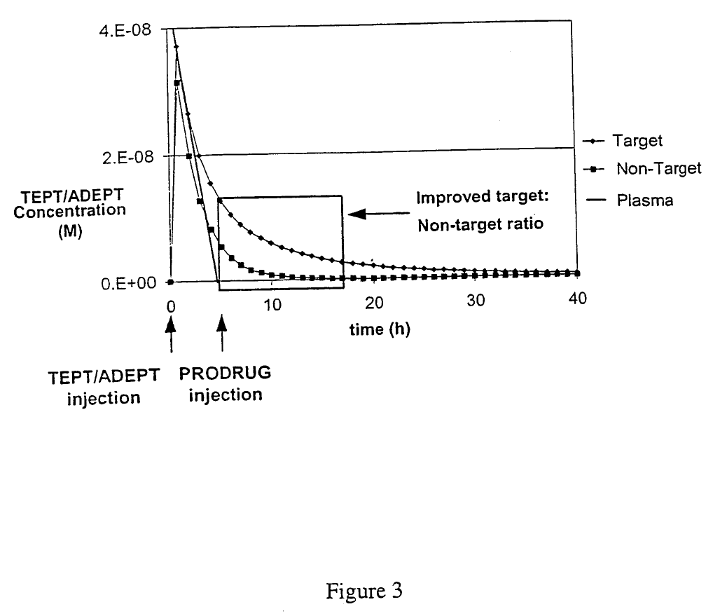

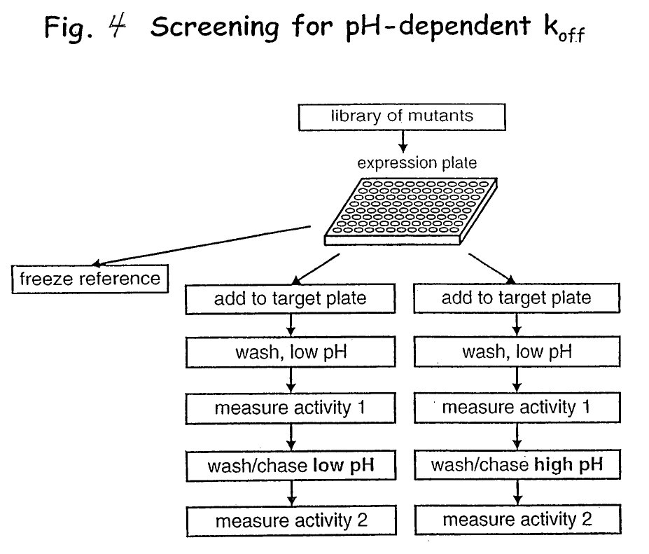

In another embodiment, a dosing strategy is used to broaden the therapeutic window provided by the intrinsic ratio of expression of the microtarget on target versus non-target tissue. For example, as shown in FIG. 3, administration of the prodrug can be delayed to a time after TEPT or ADEPT MDTA dosing where the target to non-target ratio of MDTA is increased and the concentration of the MDTA at the target is adequate for efficacy. This dosing strategy is not available to the conventional immunotoxins.

Examples of enzyme/prodrug/active drug combinations are found in, e.g., Bagshawe et al., Current Opinions in Immunology, 11:579-83 (1999); Wilman, “Prodrugs In Cancer Chemotherapy,” Biochem Society Transactions, 14, pp. 375-82 (615th Meeting, Belfast 1986) and V. J. Stella et al., “Prodrugs: A Chemical Approach To Targeted Drug Delivery,” Directed Drug Delivery, R. Borchardt et al. (ed), pp.247-67 (Humana Press 1985). In one embodiment, the prodrug is a peptide. Examples of peptides as prodrugs can be found in Trouet et al., Proc Natl Acad Sci USA 79:626 (1982), and Umemoto et al., Int J Cancer 43:677 (1989). These and other reports show that peptides are sufficiently stable in blood. Another advantage of peptide-derived prodrugs is their amino acid sequences can be chosen to confer suitable pharmacological properties like half-life, tissue distribution, and low toxicity to the active drugs. Most reports of peptide-derived prodrugs relied on relatively nonspecific activation of the prodrug by, for instance, lysosomal enzymes. Recently, it was reported that a peptide-drug conjugate was specifically cleaved by prostate specific antigen (PSA) at a tumour site. See DeFeo-Jones et al., Nat Med 6:1248 (2000). This report shows the activation of peptide prodrugs at the tumor site is an efficient way to increase the selectivity of an anticancer agent.

The prodrug can be one that is converted to an active drug in more than one step. For example, the prodrug can be converted to a precursor of an active drug by the MDTA. The precursor can be converted into the active drug by, for example, the catalytic activity of one or more additional MDTAs, the catalytic activities of one or more other enzymes administered to the subject, the catalytic activity of one or more enzymes naturally present in the subject or at the target site in the subject (e.g., a protease, a phosphatase, a kinase or a polymerase), by a drug that is administered to the subject, or by a chemical process that is not enzymatically catalyzed (e.g., oxidation, hydrolysis, isomerization, epimerization).

Drugs

Most studies involving prodrugs are generated after programs with existing drugs are found to be problematic. In particular anticancer drugs were generally characterized by a very low therapeutic index. By converting these drugs into prodrugs with reduced toxicity and then selectively activating them in the diseased tissue, the therapeutic index of the drug was significantly increased. See, e.g., Melton et al., Enzyme-prodrug strategies for cancer therapy (1999), and Niculescu-Duvaz et al., Anticancer Drug Des 14:517 (1999).

The literature describes many methods to alter the substrate specificity of enzymes by protein engineering, or directed evolution. Thus one skilled in the art is able to evolve the specificity of an enzyme to accommodate even structures that would be poor substrates for naturally-occurring enzymes. Accordingly, prodrugs can be designed even though the drugs were otherwise not amenable to a prodrug strategy.

Curnis et al., Nat Biotechnol 18:1185 (2000) showed the cytokine TNFα, when selectively targeted towards tumor vasculature, exhibited a strong antitumor effect. Otherwise, systemic delivery of TNFα is hampered by its toxicity. Other cytokines are likely to have similar limitations. The present invention enables the design of cytokine-based prodrugs that are selectively activated in diseased tissue by a MDTA.

A number of studies have been performed with toxins coupled to targeting agents (usually antibodies or antibody fragments). See, e.g., Torchilin, Eur J Pharm Sci 11 Suppl 2:S81 (2000) and Frankel et al., Clin Cancer Res 6:326 (2000). An alternative to the above is to convert these toxins into prodrugs and then selectively release them in the diseased tissue.

Prodrugs

The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide containing prodrugs, 5-fluorocytosine and other 5-fluorouridine prodrugs which can be converted by the enzyme of the conjugate into the more active cytotoxic free drug. Examples of cytotoxic drugs that can be derivatized into a prodrug form for use in this invention include, but are not limited to, etoposide, temposide, adriamycin, daunomycin, carminomycin, aminopterin, dactinomycin, mitomycins, cis-platinum and cis-platinum analogues, bleomycins, esperamicins (see U.S. Pat. No. 4,675,187), 5-fluorouracil, melphalan, other related nitrogen mustards, and derivatives thereof. See, e.g., U.S. Pat. No. 4,975,278.

In one embodiment of the invention, the MDTA comprises an alkaline phosphatase (AP) that converts a 4′-phosphate derivative of the epipodophyl-lotoxin glucosides into an active anti-cancer drug. Such derivatives include etoposide-4′-phosphate, etoposide-4′-thiophosphate and teniposide-4′-phosphate. Other embodiments of the invention may include phosphate derivatives of these glucosides wherein the phosphate moiety is placed at other hydroxyl groups on the glucosides. According to another embodiment, however, the phosphate derivative used as a pro-drug in this invention is etoposide-4′-phosphate or etoposide-4′-thiophosphate. The targeted AP removes the phosphate group from the prodrug, releasing an active antitumor agent. The mitomycin phosphate prodrug of this embodiment may be an N⁷-C₁₋₈ alkyl phosphate derivative of mitomycin C or porfiromycin, or pharmaceutically acceptable salts thereof. N⁷ refers to the nitrogen atom attached to the 7-position of the mitosane nucleus of the parent drug. According to another embodiment, the derivative used is 7-(2′-aminoethylphosphate)mitomycin (“MOP”). Alternatively, the MOP compound may be termed, 9a-methoxy-7-[[(phos-phonooxy)ethyl]amino]mitosane disodium salt. Other embodiments of the invention may include the use pf N⁷-alkyl mitomycin phosphorothioates as prodrugs.

In still another embodiment of the invention, the MDTA comprises a penicillin amidase enzyme that converts a novel adriamycin prodrug into the active antitumor drug adriamycin. In another embodiment, the penicillin amidase is a penicillin amidase (“PVA”) isolated from Fusarium oxysporum that hydrolyzes phenoxyacetyl amide bonds. The prodrug utilized can be N-(p-hydroxyphenoxyacetyl)adriamycin (“APO”), which is hydrolyzed by the amidase to release the potent antitumor agent, adriamycin

The present invention also comprises, for example, the use of the adriamycin prodrug, N-(p-hydroxyphenoxyacetyl)adriamycin and other related adriamycin prodrugs that can be derivatized in substantially the same manner. For example, use of the prodrug N-(phenoxyacetyl) adriamycin is also within the scope of the invention. In addition, it is to be understood that the adriamycin prodrugs of this invention include other N-hydroxyphenoxyacetyl derivatives of adriamycin, e.g., substituted at different positions of the phenyl ring, as well as N-phenoxyacetyl derivatives containing substituents on the phenyl ring other than the hydroxyl group described herein.

Furthermore, the present embodiment encompasses the use of other amidases, such as penicillin G amidase, as part of the MDTA as well as other prodrugs correspondingly derivatized such that the particular amidase can hydrolyze that prodrug to an active antitumor form. For example, when the MDTA comprises penicillin G amidase, the prodrug should contain a phenylacetylamide group (as opposed to the phenoxyacetylamide group of APO) because penicillin G amidases hydrolyze this type of amide bond (see, e.g., A. L. Margolin et al., Biochim. Biophys Acta. 616, pp. 283-89 (1980)). Thus, other prodrugs of the invention include N-(p-hydroxyphenylacetyl)adriamycin, N-(phenylacetyl)adriamycin and other optionally substituted N-phenylacetyl derivatives of adriamycin.

It should also be understood that the present invention includes any prodrug derived by reacting the amine group of the parent drug with the carboxyl group of phenoxyacetic acid, phenylacetic acid or other related acids. Thus, prodrugs of anthracyclines other than adriamycin that are capable of being derivatized and acting in substantially the same manner as the adriamycin prodrugs described herein falls within the scope of this invention. For example, other prodrugs that can be produced and used in accordance with this invention include hydroxyphenoxyacetylamide derivatives, hydroxypbenylacetylamide derivatives, phenoxyacetylamide derivatives and phenylacetylamide derivatives of anthracyclines such as daunomycin and carminomycin. Other amine-containing drugs such as melphalan, mitomycin, aminopterin, bleomycin and dactinomycin can also be modified described herein to yield prodrugs of the invention.

Yet another embodiment of the invention involves a MDTA form of the enzyme cytosine deaminase (“CD”). The deaminase enzyme catalyzes the conversion of 5-fluorocytosine (“5-FC”), a compound lacking in antineoplastic activity, to the potent antitumor drug, 5-fluorouracil (“5-FU”).

Another embodiment of the method of this invention provides a method of combination chemotherapy using several prodrugs and a single MDTA. According to this embodiment, a number of prodrugs are used that are all substrates for the same MDTA. Thus, a particular MDTA converts a number of prodrugs into cytotoxic form, resulting in increased antitumor activity at the tumor site.

According to another embodiment, a number of different MDTAs are used. Each MDTA can be used to convert its respective prodrug or prodrugs into active form at the target tumor site.

Still another embodiment of this invention involves the use of a number of MDTAs wherein the target bound by the enzymes varies, i.e., a number of MDTAs are used, each one binding specifically to a different target of interest. The catalytic activities of the MDTAs may be the same or may vary. This embodiment may be especially useful in situations where, for example, the amounts of the various targets on the surface of a tumor is unknown and one wants to be certain that sufficient enzyme is targeted to the tumor site. The use of a number of MDTAs recognizing different targets on the tumor increases the likelihood of obtaining sufficient enzyme at the tumor site for conversion of a prodrug or series of prodrugs. Additionally, this embodiment is important for achieving a high degree of specificity for the tumor because the likelihood that normal tissue will possess all of the same tumor-associated antigens is small (cf, I. Hellstrom et al., “Monoclonal Antibodies To Two Determinants Of Melanoma-Antigen p97 Act Synergistically In Complement-Dependent Cytotoxicity”, J. Immunol, 127 (No. 1), pp. 157-160(1981)).

In another embodiment, a MDTA is used that binds to a plurality of targets on a diseased cell. In another embodiment, the MDTA comprises a plurality of targeting sites, each of which binds to a different target on the diseased cell. The MDTA binds relatively weakly to cells having fewer than all of the targets but relatively strongly to cells having all of the targets.

There is often a requirement for extending the blood circulation half-lives of pharmaceutical peptides, proteins, or small molecules. Typically short half-lives—lasting minutes to hours-require not only frequent, but also high, doses for therapeutic effect—often so high that initial peak doses cause side effects. Extending the half-life of such therapeutics permits lower, less frequent, and therefore potentially safer doses, which are cheaper to produce. Previously researchers have increased protein half-life by fusing them covalently to PEG, see U.S. Pat. No. 5,711,944, human blood serum albumin, see U.S. Pat. No. 5,766,883, or Fc fragments, see WO 00/24782. In addition, nonspecific targeting of drugs to human serum albumin has been accomplished by chemical coupling drugs in vivo. See U.S. Pat. No. 5,843,440. Furthermore, in the case of cancer drugs it has been proposed that high molecular weight drugs may localize in tumors due to enhanced permeability and retention. Therefore, improvement in the therapeutic index of a drug can be obtained by linking the drug to a protein or other high molecular weight polymer.

However, the prior art methods for stabilizing protein and peptide therapeutics or increasing the size of cancer therapeutics have several limitations. These methods suffer from the lack of specificity involved in chemical coupling. There is also an inherent limitation of C— and N-terminal fusions in the case of fusion peptides since only two sites of attachment are possible. In addition, protein production of HSA conjugates can be problematic on a large scale. There is little or no release of covalently fused therapeutics so the pharmacodynamic properites of the therapeutic construct are not easily controlled. In addition, all of these methods substantially increase the time and effort required to identify stable therapeutics since they are not modular in nature.

In one embodiment, the present invention provides a method to selectively stabilize a therapeutic peptide, protein, or small molecule by non-covalently targeting the therapeutic site specifically to human serum albumin (HSA). Using selective targeting methods, peptide sequences that selectively bind to serum albumin with high affinity and high selectivity could be identified. Briefly, HSA-depleted blood is incubated with a library of molecules, preferably peptides. Peptides that do not bind to HSA-depleted blood are then incubated with immobilized HSA, washed extensively, and HSA binding peptides are then identified. Using the methods described in the current invention one can make the interaction between HSA and the peptide milieu-dependent such that the peptide strongly interacts with HSA in most tissues but interacts only weakly with HSA in a target tissue. As a consequence, such an MDTA will be transported with high efficiency and long, slow clearance through the blood steam and will be selectively released in the target tissue. Of particular interest are MTDAs that bind to HSA with high affinity at normal pH (approximately 7.4) but with weaker affinity at lower pH. These peptides can be further optimized for use as a therapeutic, e.g., to limit their immunological response, proteolytic susceptiblity in the blood, or ease of manufacture. Incorporation of these small peptides into an MDTA can substantially increase the half-life or therapeutic index of the MDTA. Furthermore, protease clip sites can be introduced between the HSA targeting peptide and the active moiety or other part of the MDTA. When these HSA targeted MDTAs are administered in the blood, they selectively bind to HSA and could be released based upon the physically designed properties of the binding agent (kₒₙ & k_{off} in the blood) or by enzymatic cleavage or activation. MDTAs of this type are not limited to those that bind HSA; any component of blood, e.g., long lived blood proteins including Fc fragments, α2-macroglobulin, steroids, and erythrocytes, can be exploited similarly. See Patent Cooperation Treaty Application WO 01/45746 A2, incorporated herein by reference in its entirety.

The vasculature in cancer tissue exhibits a higher than normal diffusivity. See Yuan et al., Cancer Res 55:3752 (1995). Furthermore, the diffusivity of macromolecules in the interstitial space of tumors is relatively high compared to normal tissues. See Jain, Cancer Res 47:3039 (1987).

A recent review summarizes experimental results that demonstrate that the increased diffusivity of tumors can be exploited by designing macromolecular prodrugs in particular based an coupling to PEG. See Greenwald et al., Crit Rev Ther Drug Carrier Syst 17:101 (2000). However, these prodrugs rely for their activation either on chemical lability of the linker or on rather non-specific enzymes in the tumor site. This approach can be significantly enhanced by employing an MDTA that binds with high affinity to its carrier in normal tissues but it binds with low affinity to its carrier in the milieu of the diseased tissue.

In another embodiment the present invention provides a method of treating a condition in subject comprising administering to the subject a MDTA with β-lactamase activity and a prodrug. In another embodiment, the MDTA is targeted to cancerous cell, tissue, tumor or organ. In another embodiment, the cancer is a melanoma or a carcinoma. In another embodiment, the prodrug is converted by the MDTA into an active drug. In another embodiment, the active drug is an alkylating agent. In another embodiment, the prodrug is an anticancer nitrogen mustard prodrug. In another embodiment, the active drug is melphalan. In another embodiment, the prodrug is C-Mel. See Kerr et al., Bioconjugate Chem. 9:255-59 (1998). In another embodiment, the prodrug is vinca-cephalosporin or doxorubicin cephalosporin. See Bagshawe et al., Current Opinion in Immunology, 11:579-83 (1999). Other prodrug/enzyme combinations that can be used in the present invention include, but are not limited to, those found in U.S. Pat. No. 4,975,278 and Melton et al., Enzyme-Prodrug Strategies for Cancer Therapy Kluwer Academic/Plenum Publishers, New York (1999).

The list of candidates for the pro-part of the prodrugs is extensive and diverse, and many are well known to those of skill in the art.

Pharmaceutical Compositions

The MDTAs, nucleic acids encoding them, and, in certain embodiments, prodrugs (also referred to herein as “active compounds”) described herein can be incorporated into pharmaceutical compositions suitable for administration. Such compositions typically comprise the active compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a MDTA, prodrug (or its corresponding active drug) or nucleic acid of interest. Such methods comprise formulating a pharmaceutically acceptable carrier with an agent which modulates expression or activity of an active compound of interest. Such compositions can further include additional active agents. Thus, the invention further includes methods for preparing a pharmaceutical composition by formulating a pharmaceutically acceptable carrier with an agent that modulates expression or activity of a MDTA, prodrug (or its corresponding active drug) or nucleic acid of interest and one or more additional active compounds.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

As defined herein, a therapeutically effective amount of a MDTA (i.e., an effective dosage) is the amount of the MDTA that is administered to a subject to produce a desired therapeutic effect in the subject. In the case of MDTAs to be used as part of MDTA prodrug therapy applications, a therapeutically effective amount of the MDTA is an amount sufficient to convert enough prodrug to active drug that a symptom of the disorder being treated is ameliorated.

Typically, the amount of MDTA to be delivered to a subject will depend on a number of factors, including, for example, the route of administration, the activity of the MDTA, the degree to which it is specifically targeted to the desired cells, tissues or organs of the subject, the length of time required to clear the non-specifically bound MDTA from the subject, the desired therapeutic effect, the body mass of the subject, the age of the subject, the general health of the subject, the sex of the subject, the diet of the subject, the subject's immune response to the MDTA, other medications or treatments being administered to the subject, the severity of the disease and the previous or future anticipated course of treatment.

For applications in which a prodrug also is administered, other factors affecting the determination of a therapeutically effective dose will include, for example, the amount of prodrug administered, the activity of the prodrug and its corresponding active drug, and the side effects or toxicities of the prodrug and the active drug.

Examples of ranges of mass of MDTA/mass of subject include, for example, from about 0.001 to 30 mg/kg body weight, from about 0.01 to 25 mg/kg body weight, from about 0.1 to 20 mg/kg body weight, and from about 1 to 10 mg/kg, 2 to 9 mg/kg, 3 to 8 mg/kg, 4 to 7 mg/kg, or 5 to 6 mg/kg body weight.

In a particular example, a subject is treated with a MDTA in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of MDTA may increase or decrease over the course of a particular treatment, and that the treatment will continue, with or without modification, until a desired result is achieved or until the treatment is discontinued for another reason. Changes in dosage may result and become apparent from the results of diagnostic assays as described herein.

In one embodiment, administration of MDTA is systemic. In another embodiment, administration of MDTA is at or near the target to be bound.

In an embodiment of the present invention, a prodrug also is administered to the subject. It is understood that appropriate doses of prodrugs depend upon a number of factors within the ken of the ordinarily skilled physician, veterinarian, or researcher. The dose(s) of the prodrug will depend, for example, on the same factors provided above as factors affecting the effective dose of the MDTA. Exemplary doses include milligram or microgram amounts of the prodrug per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a prodrug depend upon the potency of the prodrug with respect to the desired therapeutic effect. When one or more of these prodrugs is to be administered to an animal (e.g., a human), a physician, veterinarian, or researcher may, for example, prescribe a relatively low dose at first, subsequently increasing the dose until an appropriate response is obtained.

The timing of administration of the prodrug is another important factor to be considered, as illustrated in FIG. 3. Preferably, the MDTA is administered to the subject, then the prodrug is administered. More preferably, the time between the administration of the MDTA and administration of the prodrug is sufficient to allow the MDTA to accumulate at its target site by binding to its target, and to allow unbound MDTA to be cleared from the non-targeted portions of the subject's body. Most preferably, the ratio of target-bound MDTA to unbound MDTA in the subject's body will be at or near its maximum when the prodrug is administered. The time necessary after administration of the MDTA to reach this point is called the clearing time. The clearing time can be determined or approximated in an experimental system by, for example, administering a detectable MDTA (e.g., a radiolabeled or fluorescently labeled MDTA) to a subject and simultaneously measuring the amount of enzyme at the target site and at a non-targeted control site at timed intervals. For some prodrugs, particularly those whose counterpart active drugs are highly toxic, it may be more important to ensure that the levels of unbound MDTA in the subject's system are below a certain threshold. This too can be determined experimentally, as described above.

In one embodiment, administration of the prodrug is systemic. In another embodiment, administration of the prodrug is at or near the target to be bound.

The pharmaceutical compositions can be included in a container, pack, dispenser or kit together with instructions for administration.

Manipulation of a Compartment

The present invention also provides manipulation of a compartment (e.g., target tissue, organ and/or tumor, among others) to facilitate better selectivity, binding and/or retention of a targeting moiety within the compartment for isolation and/or selection of a MDTA. When the compartment is manipulated, it can be made more or less responsive to effectors (e.g., molecules, environment and/or stressors) and facilitate better targeting of the preferred compartment by agents. Accordingly, these methods may also be used as part of MDTA therapeutic applications of the MDTA, such as cancer therapy or other disease pathologies.

In one embodiment, the permeability of the compartment can be manipulated to allow more efficient access of a targeting molecule into the compartment or to allow increased retention of the targeting molecule in the compartment. For example, the vascular permeability of the compartment may be increased thereby increasing the rate of diffusion of a targeting agent into the compartment; or, the rate of fluid or solute efflux from the compartment may be decreased, thereby decreasing the rate of release or egress of a targeting moiety from the compartment (see, Lammerts et. al., Interference with TGF-beta1 and -beta3 in tumor stroma lowers tumor interstitial fluid pressure independently of growth in experimental carcinoma, Int J Cancer. Dec. 10, 2002;102(5):453-62). Vasoactive agents that can cause vascular leak (Iversen V V, Reed R K., PGE1 induced transcapillary transport of 51Cr-EDTA in rat skin measured by microdialysis. Acta Physiol Scand. December 2002; 176(4):269-74.), vasodilitation, vasoconstriction, decreased vascular leakage, increased compartment blood flow, increased or decreased blood pressure or a combination can be used to manipulate the compartment (see, for example, The Human Cardiovascular System, J. T. Shepherd and P. M. Vanhoutte, Eds, Raven Press, 1979, pp 181-207).

In another embodiment, agents can be used to manipulate the milieu to affect other compartments (e.g., peripheral compartments) to minimize targeting moiety influx and/or residence time (the duration of time that a pharmacodynamically or biologically or functionally relevant amount of the MDTA is retained or remains in the preferred compartment) in the other compartments.

In another embodiment, manipulation includes altering target density, amount, distribution, turnover or subtype to allow increased or decreased MDTA binding or to allow increased MDTA residence time, on or in the compartment. For example, the compartment may be exposed or conditioned with molecules or environmental modifications that cause a redistribution or increased expression of the specific target, e.g., target redistributing from cytoplasm to cell surface (J Steroid Biochem Mol Biol April 2003;84(5):527-536 Myometrial effects of selective estrogen receptor modulators on estradiol-responsive gene expression are gene and cell-specific. Farnell Y Z, Ing N H.), target redistributing as a result of decreased cell surface internalization or shedding (Prete S P, Cappelletti D, Baier S, Nasuti P, Guadagni F, De Vecchis L, Greiner J W, Bonmassar E, Graziani G, Aquino A. Int Immunopharmacol April 2002;2(5):641-51) or target redistributing from one histological region less accessible to the MDTA to another histological region more accessible to the MDTA due to increased locoregional target density (Agonist-induced capping of adhesion proteins and microparticle shedding in cultures of human renal microvascular endothelial cells. Jy W, Jimenez J J, Mauro L M, Ahn Y S, Newton K R, Mendez A J, Arnold P I, Schultz D R.) or MDTA accessibility by, for example, agents that modulate the differentiation or tissue architecture or response of the preferred compartment to various effectors (e.g., retinoids, see Ohannesian D W, Lotan D, Lotan R. Cancer Res Nov. 15, 1994;54(22):5992-6000, butyrates and other related agents Toribara, N. W., Sack, T. L., Gumm, J. R., Ho, S. B., Shively, J. E., Wilson, J. K. V., and Kim, Y. Cancer Res. 49: 3321-3327, 1989). Target density could also be affected by induction of increased production of the target with various effectors. (e.g., upregulation of CEA expression by chemotherapeutics, Correale, P., Aquino, A., Giuliani, A., Pellegrini, M., Micheli, L., Cusi, M. G., Nencini, C., Petrioli, R., Trete, S., De Vecchis, L., Turriziani, M., Giorgi, G., Monmasser, E., and Francini, G. Int. J. Cancer, 104:437-445, 2003., cytokines—Kondo, H., Tanaka, N., Naomoto, Y., and Orita, K. Jpn J. Cancer Res. 78: 1258, 1987.Toth, C. A., and Thomas, P., J. Interferon Res. 10: 579-588, 1990.) and signal transduction effectors, particularly kinase effectors such as the tyrosine kinase inhibitor staurosporine (Prete S P, Cappelletti D, Baier S, Nasuti P, Guadagni F, De Vecchis L, Greiner J W, Bonmassar E, Graziani G, Aquino A. Int Immunopharmacol April 2002;2(5):641-51).

In another embodiment, compartment milieu can be manipulated to augment binding affinity and/or decrease dissociation rates, for example, by modulating the pH, tonicity and/or temperature, among other things, of the compartment. This could be accomplished by: changing the local temperature (Schaffer M, Krych M, Pachmann S, Abdel-Rahman S, Schaffer P M, Ertl-Wagner B, D hmke E, Issels R D., Feasibility and morbidity of combined hyperthermia and radiochemotherapy in recurrent rectal cancer), preliminary results isolating zones of perfusion (Radiother Oncol February 1999;50(2):215-23, Radiosensitization by intratumoral administration of cisplatin in a sustained-release drug delivery system. Ning S, Yu N, Brown D M, Kanekal S, Knox S J.), whole body exposure to altered atmospheric conditions (Am J Physiol Cell Physiol February 2000;278(2):C292-302 Hyperbaric oxygen downregulates ICAM-1 expression induced by hypoxia and hypoglycemia: the role of NOS. Buras J A, Stahl G L, Svoboda K K, Reenstra W R.) or parenteral or topical administration of agents that elicit a desired pH or tonicity shift selectively in the compartment milieu versus other compartments. One skilled in the art may be able to conceive of additional methods of manipulation intended to be within the scope of the present invention.

### EXAMPLE 1

### SGN17 His Scan Method

This example demonstrates that a non-milieu dependent targeting agent can be modified to generate a milieu-dependent targeting agent.

pADEPT06 DNA Template:

This plasmid is 5.2 kb and encodes a single chain antibody variable region fragment (scFv) fused to β-lactamase (BLA) with a pelB leader sequence, and is driven by a lac promoter (P lac) (FIG. 1). The plasmid also carries a chloramphenicol resistance gene (CAT) as a selectable marker. This particular SGN17 plasmid was made by a 3-piece ligation utilizing a linker. Two plasmids were used to make pADEPT06: pCB04 for the vector fragment with the pel B leader sequence, and pCR13 for the scFv-b1a gene. pCBO4 was digested with HindIII and DraIII (both from New England Biolabs, Beverly, Mass.) resulting in a 2.7 kb fragment with the pCB04 backbone. pCR13 was digested with NdeI (Roche Molecular Biochemicals, Indianapolis, Ind.) and DraIII resulting in the 2.4 kb fragment containing the fusion protein with the pelB leader sequence. Digests pCR13 were done in NEB2 buffer from NEB (50 nM NaCl, 10 mN Tris-HCl, 10 mM MgCl₂, 1 mM dithiothreitol (pH 7.9 at 25° C.). Both fragments were gel purified from 1% agarose gel using a Qiagen kit (Qiagen, Valencia, Calif.). A linker sequence with 5′ HindIII complementary ends and 3′ NdeI complementary ends was used to link the 2.7 kb fragment and the 2.4 kb fragment upstream of the pel B leader sequence. The pCB04 fragment was combined with the pCR13 fragment and the linker in a 1:1:10 molar ratio (respectively), using 17 μl DNA volume (95 ng total DNA) and 17 μl Takara ligase solution I (Panvera, Madison, Wis.) and incubated overnight at 16° C. in a PTC-200™ machine (MJ Research, Waltham, Mass.). Sequencing information shows that the linker region is repeated upstream of the leader sequence.

Mutagenic Primers:

Overlapping mutagenic primers were designed to replace certain amino acids with histidine residues in the CDR3 regions of both the heavy and light chains of the scFv portion of the scFv-BLA fusion. The wild-type codon to be mutated was changed to the codon CAT (encoding histidine) in a pair of primers. The mutated codon in each primer was flanked on each side by 17 nucleotides of wild-type sequence, unless the primer ended in a stretch of A residues; in this case, the flanking sequence was extended so that it ended with a G or C residue. Each primer was designed so that its mutant codon had the same number of nucleotides flanking it on each side. Each primer was named according to the mutation it was designed to create. For example, HCL100F is the forward primer for the heavy chain (HC) mutating the Leucine (L) in position 100. Its overlapping primer is called HCL100R.

The names and sequences of the mutagenic oligos are provided in Table 1.

TABLE 1
SGN17 His Scan Primers

| | |
|---|---|
| Heavy Chain | |
| HCK64F | ACTACAATCCATCTCTCCATAGTCGCATTTCCATCAC |
| HCK64R | GTGATGGAAATGCGACTATGGAGAGATGGATTGTAGT |
| HCR97F | GCCACATATTACTGTGCACATAGGACTCTGGCTACTTAC |
| HCR97R | GTAAGTAGCCAGAGTCCTATGTGCACAGTAATATGTGGC |
| HCR98F | CATATTACTGTGCAAGACATACTCTGGCTACTTACTA |
| HCR98R | TAGTAAGTAGCCAGAGTATGTCTTGCACAGTAATATG |
| HCT99F | ATTACTGTGCAAGAAGCCATCTGCCTACTTACTATGC |
| HCT99R | GCATAGTAAGTAGCCAGATGCCTTCTTGCACAGTAAT |
| HCL100F | ACTGTGCAAGAAGGACTCATGCTACTTACTATGCTAT |
| HCL100R | ATAGCATAGTAAGTAGCATGAGTCCTTCTTGCACAGT |
| HCA101F | GTGCAAGAAGGACTCTGCATACTTACTATGCTATGGA |
| HCA101R | TCCATAGCATAGTAAGTATGCAGAGTCCTTCTTGCAC |
| HCT102F | CAAGAAGGACTCTGGCTCATTACTATGCTATGGACTA |
| HCT102R | TAGTCCATAGCATAGTAATGAGCCAGAGTCCTTCTTG |
| HCY103F | GAAGGACTCTGGCTACTCATTATGCTATGGACTACTG |
| HCY103R | CAGTAGTCCATAGCATAATGAGTAGCCAGAGTCCTTC |
| HCY104F | GGACTCTGGCTACTTACCATGCTATCGACTACTGGGG |
| HCY104R | CCCCAGTACTCCATAGCATGCTAAGTAGCCAGAGTCC |
| HCA105F | CTCTGGCTACTTACTATCATATGGACTACTGGGGTCA |
| HCA105R | TGACCCCAGTAGTCCATATGATAGTAAGTAGCCAGAG |
| HCM106F | TGGCTACTTACTATGCTCATGACTACTGGGGTCAAGG |
| HCM106R | CCTTGACCCCAGTAGTCATGAGCATAGTAAGTAGCCA |
| HCD107F | CTACTTACTATGCTATGCATTACTGGCGTCAAGGAAC |
| HCD107R | GTTCCTTGACCCCAGTAATGCATAGCATAGTAAGTAG |
| HCY108F | CTTACTATGCTATGGACCATTGGGGTCAAGGAACCTC |
| HCY108R | GAGGTTCCTTGACCCCAATGGTCCATAGCATAGTAAG |
| HCW109F | ACTATGCTATGGACTACCATGGTCAAGGAACCTCTGT |
| HCW109R | ACAGAGGTTCCTTGACCATGGTAGTCCATAGCATAGT |
| Light Chain | |
| LCR54F | CAAAGCTCCTGATCTACCATGTTTCCAACCGATTTTC |
| LCR54R | GAAAATCGGTTGGAAACATGGTAGATCAGGAGCTTTG |
| LCR58F | GATTTTCTGGGGTCCCAGACCATTTCAGTGGCAGTGGATCAGG |
| LCR58R | CCTGATCCACTGCCACTGAAATGGTCTGGGACCCCAGAAAATC |
| LCQ94F | GAGTTTATTTCTGCTCTCATAGTACACATGTTCCTCC |
| LCQ94R | CGAGGAACATGTGTACTATGAGAGCAGAAATAAACTC |
| LCS95F | GTTTATTTCTGCTCTCAACATACACATGTTCCTCCGACG |
| LCS95R | CGTCGGAGGAACATGTGTATGTTGAGAGCAGAAATAAAC |
| LCT96F | GTTTATTTCTGCTCTCAAAGTCATCATCTTCCTCCGACGTTCGGT |
| LCT96R | ACCGAACGTCGGAGGAACATGATGACTTTGAGAGCAGAAATAAAC |
| LCH97F | TCTGCTCTCAAACTACACATGTTCCTCCGACGTTCGG |
| LCH97R | CCGAACGTCCGAGGAACATCTGTACTTTGAGAGCACA |
| LCV98F | GCTCTCAAAGTACACATCATCCTCCGACGTTCGGTGG |
| LCV98R | CCACCGAACGTCGGAGGATGATGTGTACTTTGAGAGC |
| LCP99F | CTCAAAGTACACATGTTCATCCGACCTTCGGTGGAGG |
| LCP99R | CCTCCACCGAACCTCGGATGAACATCTGTACTTTGAC |
| LCP100F | CAAACTACACATCTTCCTCATACGTTCGGTGGAGGCACC |
| LCP100R | GGTGCCTCCACCGAACGTATGAGGAACATGTGTACTTTG |
| LCT101F | AGTACACATCTTCCTCCGCATTTCGGTGGAGGCACCAAG |
| LCT101R | CTTGGTGCCTCCACCGAAATGCGGAGGAACATGTGTACT |

All sequences written 5′-3′. Mutagenic codon in bold and underlined.

A QUICKCHANGE™ site directed mutagenesis kit (Stratagene, La Jolla, Calif.) was used to set up PCR amplifications as follows:

| | | |
|---|---|---|
| H₂O | 39 | μl |
| 10x buffer | 5 | μl |
| dNTP mix | 1.5 | μl |
| Forward primer | 1 | μl (0.5 μM final concentration) |
| Reverse primer | 1 | μl (0.5 μM final concentration) |
| pfu polymerase | 1 | μl |
| Plasmid DNA | 1.5 | μl (150 ng) |
| Total | 50 | μl |

The buffer comprised 100 mM KCl, 100 mM (NH₄)SO₄, 200 mM Tris-HCl (pH 8.8), 20 mM MgSO₄, 1% Triton X-100, 1 mg/ml nuclease-free bovine serum albumin (BSA).

The following touchdown PCR program was used in a PTC-200T machine (MJ Research, Waltham, Mass.):
1) 95° C., 2 minutes
2) 95° C., 45 seconds
3) 60° C., 1 minute (Reduced by 1.0° C. per cycle)
4) 68° C. 11 minutes (i.e., 2 minutes per kb, 5 kb plasmid, plus an additional minute)
5) Go to step (2) for 9 cycles
6) 95° C., 45 seconds
7) 50° C., 1 minute
8) 68° C., 11 minutes
9) Go to step (6) for 5 cycles
10) Hold at4° C.

A negative control without primers was also set up and carried through all steps.

DpnI Digest:

DpnI is a restriction enzyme that cuts methylated and hemimethylated, but not unmethylated, double-stranded DNA. After PCR, 1 μl of DpnI was added to each reaction to digest template DNA, which is methylated, but not amplified DNA, most of which is unmethylated, thus reducing the background of wild-type sequence. A sample of the control was saved before digestion. Digests were incubated at 37° C. for 1.5 hrs, then each reaction was spiked with an additional 1 μl of DpnI and incubated another 1.5 hrs. Reactions were run on a gel after digests alongside the control amplification before and after DpnI digestion. All reactions appeared to work-, and, as expected, the control band was fully digested by DpnI.

Transformation:

1 μl of each reaction (not purified), including the digested control, were used to transform 50 μl of Top 10 electro-competent cells (Invitrogen, Carlsbad, Calif.) and 250 μl SOC medium (2% Bacto-Tryptone, 0.5% Bacto Yeast Extract, 10 mM NaCl, 2.5 mM KCl) was added. The cells were shaken at 37° C. for 45 min, then 30 μl of a 1 to 10 dilution was plated (i.e., one tenth of the total volume of each transformation was plated) on both 5 ppm chloramphenicol (CMP) and 5 ppm CMP+0.1 ppm cefotaxime (CTX) plates. Plates were incubated overnight at 37° C. Transformation results are provided in Table 2.

TABLE 2

| | CMP | CMP + CTX | % ACTIVE | |
|---|---|---|---|---|
| (control) | 0 | 0 | 0 | |
| ME43 | 14 | 5 | 36 | |
| ME44 | 120 | 34 | 28 | |
| ME45 | 784 | 236 | 32 | |
| ME46 | 440 | 159 | 36 | |
| ME47 | 516 | 184 | 36 | |
| ME48 | 268 | 62 | 23 | |
| ME49 | 30 | 10 | 33 | |
| ME50 | 488 | 61 | 12.5 | |
| ME51 | 316 | 57 | 18 | |
| ME52 | 380 | 192 | 50 | |
| ME53 | 440 | 80 | 18 | |
| ME54 | 968 | 308 | 32 | |
| ME55 | 356 | 148 | 42 | |
| ME56 | 90 | 17 | 19 | |
| ME57 | 424 | 112 | 26 | |
| ME58 | 38 | 10 | 26 | |
| ME59 | 141 | 53 | 38 | |
| ME60 | 212 | 144 | 68 | |
| ME61 | 90 | 27 | 30 | |
| ME62 | 268 | 87 | 32 | (WT codon) |
| ME63 | 296 | 88 | 30 | |
| ME64 | 196 | 112 | 57 | |
| ME65 | 168 | 128 | 76 | |
| ME66 | 236 | 76 | 32 | |

All bacteria transformed by and expressing a plasmid produced colonies on the CTX plate, and thus provided a measure of the efficiency of transformation. However, only bacteria transformed by plasmids containing a functional BLA grew on the CTX+CMP plates.

Clone names in Table 2 are listed in the same order as the primer pairs used to make them are listed in Table 1, e.g., ME43 was created using primer pair HCK64F/R, ME44 was created using primer pair HCR97F/R, and so on.

Four colonies were picked for each transformation (excluding LCH97 because this represents the wild-type sequence; pADEPT06 WT colonies were picked as a control). Picked colonies were first swirled into a 96 well plate with membrane bottom, each well containing 200 ul LB+5 ppm CMP, and then put into the corresponding well of another 96 well plate without filter, to be used as a stock plate.

The 96 well plates were incubated at 25° C. in a humidified box with shaking for 48 hrs. Glycerol was added to the stock plate to a final concentration of 10% and stored at −80° C.

Screening Mutants:

Target protein p97 was immobilized on a polystyrene plate by adding 100 μl of 1 μg/ml p97 in PBS and incubating the plate at 4° C. overnight. The plate was then washed with PBST (PBS+0.25% Tween 20) and blocked with 200 μl/well of 1% casein in PBS overnight at 4° C. On the day of screening, the plate was washed with PBST, then each well received 80 μl of 50 mM PBS pH7.4 and 20 μl of cell culture broth from each mutant. The plate was incubated at room temperature with gentle shaking to let SGN-17 bind to immobilized p97 on the plate. The amount of each mutant enzyme bound to p97 was determined at two time points. After 1 hour, the plate was washed with PBST, and 200 μl of the BLA substrate nitrogen in 50 mM PBS buffer pH7.4 or pH6.5 was added into each well. The amount of bound SGN-17 was measured by monitoring hydrolysis of nitrogen at wavelength 490 nm. This was the T₀ time point measurement. The plate was then incubated in each substrate buffer for one hour, providing an opportunity for bound mutant SGN-17 to dissociate, then quickly rinsed with PBST. The remaining bound SGN-17 was measured by again monitoring the hydrolysis of substrate nitrogen in each buffer. This was the T₁ time point measurement. A ratio of bound activity at T₁ vs. T₀ was calculated for each mutant, and an index was calculated by dividing the ratio of mutant over parent, as shown in Table 3.

TABLE 3

| Mutants | sequence | position | region | Index pH 7.4 | Index pH 6.5 |
|---|---|---|---|---|---|
| ME43 | K | HC62 | CDR2 | 0.61 | 0.65 |
| ME44 | R | HC94 | CDR3 | 0.24 | 0 |
| ME45 | R | HC95 | CDR3 | 0 | 0 |
| ME46 | T | HC96 | CDR3 | 0.38 | 0.09 |
| ME47 | L | HC97 | CDR3 | 0.24 | 0 |
| ME48 | A | HC98 | CDR3 | 0.49 | 0.33 |
| ME50 | Y | HC100 | CDR3 | 0.33 | 0 |
| ME51 | Y | HC101 | CDR3 | 0.26 | 0 |
| ME52 | A | HC102 | CDR3 | 0 | 0 |
| ME53 | M | HC103 | CDR3 | 0.97 | 0.8 |
| ME54 | D | HC104 | CDR3 | 0.41 | 0.7 |
| ME55 | Y | HC105 | CDR3 | 0.8 | 0.7 |
| ME56 | W | HC106 | CDR3 | 0.57 | 0.41 |
| ME58 | R | LC58 | CDR2 | 0.92 | 0.76 |
| ME59 | Q | LC94 | CDR3 | 0.28 | 0 |
| ME60 | S | LC95 | CDR3 | 1.04 | 1.09 |
| ME61 | T | LC96 | CDR3 | 0.82 | 0.81 |
| ME63 | V | LC98 | CDR3 | 0.21 | 0 |
| ME64 | P | LC99 | CDR3 | 0.35 | 0 |
| ME65 | P | LC100 | CDR3 | 0 | 0 |
| ME66 | T | LC101 | CDR3 | 1.36 | 1.73 |

A high index value for a mutant indicates that it has a slow k_{off}. An index value of 0 indicates that no binding was detected for the mutant at that pH.

These data illustrate that many residues in the CDR3s of SGN-17 can be replaced with His while retaining various degrees of binding affinity. Mutagenesis at position LC101 actually leads to an increase in binding affinity which is larger at pH 6.5 as compared to pH 7.4. Thus, the introduction of a His in position LC101 affects the pH-dependence of target binding of SGN-17. Comparing the index values at both pH values shows that several of the tested mutations affect pH-dependence of binding. Stronger effects can be achieved by adding further mutations, by testing substitutions other then His, by testing substitutions, insertions or deletions at more positions of the binding moiety, or by extending the mutagenesis to the BLA part of the fusion protein.

### EXAMPLE 2

### Affinity Maturation of an scFv by Site Saturation Scanning Mutagenesis

A. Generation of Site Saturation Libraries

64 site saturation mutagenesis libraries were generated. In each of these libraries, one codon, that codes for a CDR position (as defined by the Kabat nomenclature) in ME66.4-scFv, exactly the same as ME66, was randomized. The libraries were generated using the QuikChange protocol (Stratagene, La Jolla, Calif.) essentially as recommended by the manufacturer. Each reaction used two mutagenic oligonucleotides which had the following design: 17 perfect matches flanking the random codon on each side, NNS in place of the random codon. For example, library ME67 used the forward primer CTGGCGACTCCATCACCNNSGGTTACTGGAACTGGAT and the reverse primer ATCCAGTCCAGTAGTAACCSNNGGTGATGGAGTCGCCAG, where N represents a mixture of A, T, G, anti C and S represents a mixture of G and C. This approach allows for the generation of 32 different codons which encode all 20 amino acids. After the QuikChange reaction and Dpn I digest, which degrades parent plasmid, the reaction mixture was used to transform TOP10 cells (Invitrogen, Carlsbad, Calif.) by electroporation.

TABLE 4

oligonucleotides used to generate the 64 site
saturation libraries:

| | |
|---|---|
| Heavy | |
| Chain | |
| H31 | CTGGCGACTCCATCACCNNSGGTTACTGGAACTGGAT |
| H31 | ATCCAGTTCCAGTAACCSNNGGTGATGGAGTCGCCAG |
| H32 | GCGACTCCATCACCAGTNNSTACTGGAACTGGATCCG |
| H32 | CGGATCCAGTTCCAGTASNNACTGGTGATGGAGTCGC |
| H33 | ACTCCATCACCAGTGGTNNSTGGAACTGGATCCGGCA |
| H33 | TGCCGGATCCAGTTCCASNNACCACTGGTGATGGAGT |
| H34 | TCCATCACCAGTGGTTACNNSAACTGGATCCGGCAGTTC |
| H34 | GAACTGCCGGATCCAGTTSNNGTAACCACTGGTGATGGA |
| H50 | AACTTGAATATATGGGTNNSATAAGCGACAGTGGTAT |
| H50 | ATACCACTGTCGCTTATSNNACCCATATATTCAAGTT |
| H51 | TTGAATATATGGGTTACNNSAGCGACAGTGGTATCAC |
| H51 | GTGATACCACTGTCGCTSNNGTAACCCATATATTCAA |
| H52 | GAATATATGGGTTACATANNSGACAGTGGTATCACTTAC |
| H52 | GTAAGTGATACCACTGTCSNNTATGTAACCCATATATTC |
| H53 | TATATGGGTTACATAAGCNNSAGTGGTATCACTTACTAC |
| H53 | GTAGTAAGTGATACCACTSNNGCTTATGTAACCCATATA |
| H54 | ATGGGTTACATAAGCGACNNSGGTATCACTTACTACAAT |
| H54 | ATTGTAGTAAGTGATACCSNNGTCGCTTATGTAACCCAT |
| H55 | GTTACATAAGCGACAGTNNSATCACTTACTACAATCC |
| H55 | GGATTGTAGTAAGTGATSNNACTGTCGCTTATGTAAC |
| H56 | ACATAAGCGACAGTGGTNNSACTTACTACAATCCATC |
| H56 | GATGGATTGTAGTAAGTSNNACCACTGTCGCTTATGT |
| H57 | TAAGCGACAGTGGTATCNNSTACTACAATCCATCTCT |
| H57 | AGAGATGGATTGTAGTASNNGATACCACTGTCGCTTA |
| H58 | TAAGCGACAGTGGTATCACTNNSTACAATCCATCTCTCAAAAG |
| H58 | CTTTFGAGAGATGGATTGTASNNAGTGATACCACTGTCGCTTA |
| H59 | GACAGTGGTATCACTTACNNSAATCCATCTCTCAAAAGT |
| H59 | ACTTTTGAGAGATGGATTSNNGTAAGTGATACCACTGTC |
| H60 | GTGGTATCACTTACTACNNSCCATCTCTCAAAAGTCG |
| H60 | CGACTTTTGAGAGATGGSNNGTAGTAAGTGATACCAC |
| H61 | GTATCACTTACTACAATNNSTCTCTCAAAAGTCGCAT |
| H61 | ATGCGACTTTTGAGAGASNNATTGTAGTAAGTGATAC |
| H62 | TCACTTACTACAATCCANNSCTCAAAAGTCGCATTTC |
| H62 | GAAATGCGACTTTTGAGSNNTGGATTGTAGTAAGTGA |
| H63 | CTTACTACAATCCATCTNNSAAAAGTCGCATTTCCAT |
| H63 | CTTACTACAATCCATCTNNSAAAAGTCGCATTTCCAT |
| H64 | ACTACAATCCATCTCTCNNSAGTCGCATTTCCATCAC |
| H64 | GTGATGGAAATGCGACTSNNGAGAGATGGATTGTAGT |
| H65 | ACAATCCATCTCTCAAANNSCGCATTTCCATCACTCG |
| H65 | CGAGTGATGGAAATGCGSNNTTTGAGAGATGGATTGT |
| H97 | GCCACATATTACTGTGCANNSAGGACTCTGGCTACTTAC |
| H97 | GTAAGTAGCCAGAGTCCTSNNTGCACAGTAATATGTGGC |
| H98 | CATATTACTGTGCAAGANNSACTCTGGCTACTTACTA |
| H98 | TAGTAAGTAGCCAGAGTSNNTCTTGCACAGTAATATG |
| H99 | ATTACTGTGCAAGAAGGNNSCTGGCTACTTACTATGC |
| H99 | GCATAGTAAGTAGCCAGSNNCCTTCTTGCACAGTAAT |
| H100 | ACTGTGCAAGAAGGACTNNSGCTACTTACTATGCTAT |
| H100 | ATAGCATAGTAAGTAGCSNNAGTCCTTCTTGCACAGT |
| H101 | GTGCAAGAAGGACTCTGNNSACTTACTATGCTATGGA |
| H101 | TCCATAGCATAGTAAGTSNNCAGAGTCCTTCTTGCAC |
| H102 | CAAGAAGGACTCTGGCTNNSTACTATGCTATGGACTA |
| H102 | TAGTCCATAGCATAGTASNNAGCCAGAGTCCTTCTTG |
| H103 | GAAGGACTCTGGCTACTNNSTATGCTATGGACTACTG |
| H103 | CAGTAGTCCATAGCATASNNAGTAGCCAGAGTCCTTC |
| H104 | GGACTCTGGCTACTTACNNSGCTATGGACTACTGGGG |
| H104 | CCCCAGTAGTCCATAGCSNNGTAAGTAGCCAGAGTCC |
| H105 | CTCTGGCTACTTACTATNNSATGGACTACTGGGGTCA |
| H105 | TGACCCCAGTAGTCCATSNNATAGTAAGTAGCCAGAG |
| H106 | TGGCTACTTACTATGCTNNSGACTACTGGGGTCAAGG |
| H106 | CCTTGACCCCAGTAGTCSNNAGCATAGTAAGTAGCCA |
| H107 | CTACTTACTATGCTATGNNSTACTGGGGTCAAGGAAC |
| H107 | GTTCCTTGACCCCAGTASNNCATAGCATAGTAAGTAG |
| H108 | CTTACTATGCTATGGACNNSTGGGGTCAAGGAACCTC |
| H108 | GAGGTTCCTTGACCCCASNNGTCCATAGCATAGTAAG |
| H109 | ACTATGCTATGGACTACNNSGGTCAAGGAACCTCTGT |
| H109 | ACAGAGGTTCCTTGACCSNNGTAGTCCATAGCATAGT |
| Light | |
| Chain | |
| L24 | CCTCCATCTCTTGCAGGNNSAGTCAGAGCCTTGTACA |
| L24 | TGTACPAGGCTCTGACTSNNCCTGCAAGAGATGGAGG |
| L25 | CCATCTCTTGCAGGGCTNNSCAGAGCCTTGTACACAG |
| L25 | CTGTGTACAAGGCTCTGSNNAGCCCTGCAAGAGATGG |
| L26 | ATCTCTTGCAGGGCTAGTNNSAGCCTTGTACACAGTAAT |
| L26 | ATTACTGTGTACAAGGGTSNNACTAGCCCTGCAAGAGAT |
| L27 | CTTGCAGGGGTAGTCAGNNSCTTGTACACAGTAATGG |
| L27 | CCATTACTGTGTACAAGSNNCTGACTAGCCCTGCAAG |
| L28 | TGCAGGGCTAGTCAGAGCNNSGTACACAGTAATGGAAAC |
| L28 | GTTTCCATTACTGTGTACSNNGCTCTGACTAGCCCTGCA |
| L29 | GGGCTAGTCAGAGCCTTNNSCACAGTAATGGAAACAC |
| L29 | GTGTTTCCATTACTGTGSNNAAGGCTCTGACTAGCCC |
| L30 | CTAGTCAGAGCCTTGTANNSAGTAATGGAkACACCTA |
| L30 | TAGGTGTTTCCATTACTSNNTACAAGGCTCTGACTAG |
| L31 | TAGTCAGAGCCTTGTACACNNSAATGGAAACACCTATTTAC |
| L31 | GTAAATAGGTGTTTCCATTSNNGTGTACAAGGCTCTGACTA |
| L32 | AGAGCCTTGTACACAGTNNSGGAAACACCTATTTACA |
| L32 | TGTAAATAGGTGTTTCCSNNACTGTGTACAAGGCTCT |
| L33 | GCCTTGTACACAGTAATNNSAACACCTATTTACATTG |
| L33 | CAATGTAAATAGGTGTTSNNATTACTGTGTACAAGGC |
| L34 | TTGTACACAGTAATGGANNSACCTATTTACATTGGTA |
| L34 | TACCAATGTAAATAGGTSNNTCCATTACTGTGTACAA |
| L35 | TACACAGTAATGGAAACNNSTATTTACATTGGTACC |
| L35 | GGTACCAATGTAAATASNNGTTTCCATTACTGTGTA |
| L36 | ACAGTAATGGAAACACCNNSTTACATTGGTACCTGCA |
| L36 | TGCAGGTACCAATGTAASNNGGTGTTTCCATTACTGT |
| L37 | AGTAATGGAAACACCTATNNSCATTGGTACCTGCAGAAG |
| L37 | CTTCTGCAGGTACCAATGSMMATAGGTGTTTCCATTACT |
| L38 | ATGGAAACACCTATTTANNSTGGTACCTGCAGAAGCC |
| L38 | GGCTTCTGCAGGTACCASNNTAAATAGGTGTTTCCAT |
| L53 | CTCCAAAGCTCCTGATCNNSAGAGTTTCCAACCGATT |
| L53 | AATCGGTTGGAAACTCTSNNGATCAGGAGCTTTGGAG |
| L54 | CAAAGCTCCTGATCTACNNSGTTTCCAACCGATTTTC |
| L54 | GAAAATCGGTTGGAAACSNNGTAGATCAGGAGCTTTG |
| L55 | AGCTCCTGATCTACAGANNSTCCAACCGATTTTCTGG |
| L55 | CCAGAAAATCGGTTGGASNNTCTGTAGATCAGGAGCT |
| L56 | TCCTGATCTACAGAGTTNNSAACCGATTTTCTGGGGT |
| L56 | ACCCCAGAAAATCGGTTSNNAACTCTGTAGATCAGGA |
| L57 | TGATCTACAGAGTTTCCNNSCGATTTTCTGGGGTCCC |
| L57 | GGGACCCCAGAAAATCGSNNGGAAACTCTGTAGATCA |
| L58 | TCTACAGAGTTTCCAACNNSTTTTCTGGGGTCCCAGA |
| L58 | TCTGGGACCCCAGAAAASNNGTTGGAAACTCTGTAGA |
| L59 | ACAGAGTTTCCAACCGANNSTCTGGGGTCCCAGACAG |
| L59 | CTGTCTGGGACCCCAGASNNTCGGTTGGAAACTCTGT |
| L60 | GAGTTTCCAACCGATTTNNSGGGGTCCCAGACAGGTT |
| L60 | AACCTGTCTGGGACCCCSNNAAATCGGTTGGAAACTC |
| L94 | GAGTTTATTTCTGCTCTNNSAGTACACATGTTCCTCC |
| L94 | GGAGGAACATGTGTACTSNNAGAGCAGAAATAAACTC |
| L95 | GAGTTTATTTCTGCTCTCAANNSACACATGTTCCTCCGCATTT |
| L95 | AAATGCGGAGGAACATGTGTSNNTTGAGAGCAGAAATAAACTC |
| L96 | TATTTCTGCTCTCAAAGTNNSCATGTTCCTCCGCATTTC |
| L96 | GAAATGCGGAGGAACATGSNNACTTTGAGAGCAGAAATA |
| L97 | TCTGCTGTCAAAGTACANNSGTTCCTCCGCATTTCGG |
| L97 | CCGAAATGCGGAGGAACSNNTGTACTTTGAGAGCAGA |
| L98 | GCTCTCAAAGTACACATNNSCCTCCGCATTTCGGTGG |
| L98 | CCACCGAAATGCGGAGGSNNATGTGTACTTTGAGAGC |
| L99 | CTCAAAGTACACATGTTNNSCCGCATTTCGGTGGAGG |
| L99 | CCTCCACCGAAATGCGGSNNAACATGTGTACTTTGAG |
| L100 | CAAAGTACACATGTTCCTNNSCATTTCGGTGGAGGCACC |
| L100 | GGTGCCTCCACCGAAATGSNNAGGAACATGTGTACTTTG |
| L101 | AGTACACATGTTCCTCCGNNSTTCGGTGGAGGCACCAAG |
| L101 | CTTGGTGCCTCCACCGAASNNCGGAGGAACATGTGTACT |

B. Screen for Improved Binding

Libraries were plated onto agar plates containing LB medium and 5 mg/l chloramphenicol and 0.1 mg/l cefotaxime (Sigma). 88 colonies from each library and parent colonies were picked and inoculated into 384-well plates containing 80 ul LB containing 5 mg/l chloramphenicol and 0.1 mg/l cefotaxime. Plates were incubated at 25 C in humidified boxes with shaking for 48 hrs.

Target protein p97 (prepared as described in [Siemers, N. O., D. E. Kerr, S. Yarnold, M. R. Stebbins, V. M. Vrudhula, I. Hellrstrom, K. E. Hellstrom and P. D. Senter (1997) Bioconj Chem 8, 510-519, Construction, expression and activities of L49-sFv-beta-lactamase, a single chain antibody fusion protein for anticancer aging prodrug activation]) was immobilized in 384-well polystyrene plates by adding 40 ul of 1 ug/ml p97 in PBS and incubating the plate at 4 C overnight. The plates were then washed with PBST (PBS+0.1% Tween-20) and blocked with 200 ul/well of 1% Casein in PBS overnight at 4 C. On the day of screening, the plates were washed with PBST. Subsequently, 24 ul well of 50 mM PBS pH7.4 was first added into plate each well followed by 8 ul of cell culture broth from expression plates. The plate was incubated at room temperature with gentle shaking to let ME66-scFv to bind to immobilized P97 on the plate. After 1 hour, the plate was washed with PBST and 200 ul of BLA assay buffer containing 0.1 mg/ml nitrocefin (Oxoid, N.Y.) in 50 mM PBS buffer pH6.5 was added into each well, the bound ME66scFv was measured by monitoring hydrolysis of nitrocefin at wavelength 490 nm. The plate was then left incubated in substrate buffer to allow the bound ME66scFv-BLA to dissociate, after 1.5 hour the plate was quickly rinsed with PBST. The remaining bound ME66scFv-BLA was again measured by monitoring the hydrolysis of freshly added substrate nitrocefin. Dissociation of ME66-scFv from p97 was monitored again after 3-5 hours. A ratio of bound activity at time 1 vs. time 0 or time 2 vs. time 0 was calculated for each mutant from dissociation data, an index at each time point was further calculated by dividing ratio of mutant over parent, and winner mutants were chosen if they had a high index.

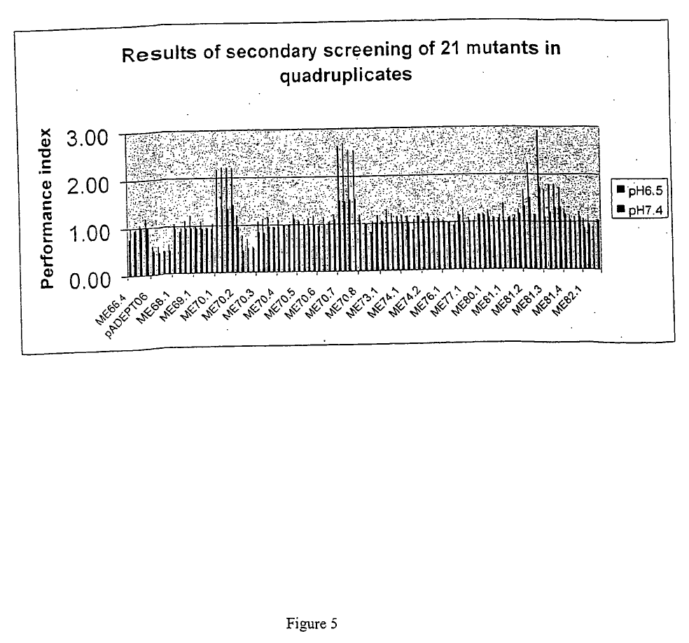

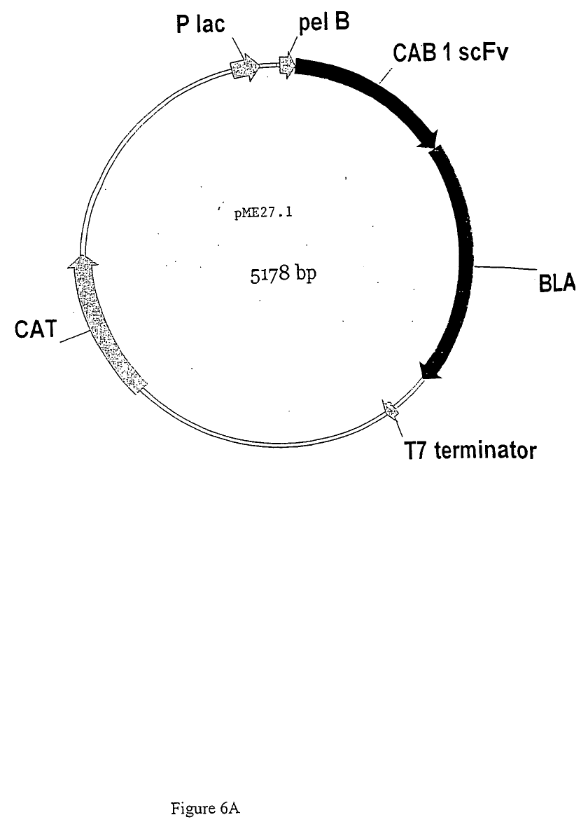

After the primary screening, 21 winners were chosen for repeat analysis in quadruplicates. Each winner was streaked out on LA agar containing 5 mg/l chworamphenicol, 4 colonies from each winner were transferred in 96 well plate containing 200 ul/well LB containing 5 mg/l chloramphenicol. Some wells were inoculated with ME66.4 as a reference. The plate was incubated at 25 C for 70 hours. Target protein p97 was bitotinylated and immobilized in 96 well neutravidin (Pierce, Rockford, Ill.) plate at a p97 concentration of 5 ug/ml of 100 ul/well, the plate was then blocked with 1% Casein. On the day of screening, 70 ul/well of PBS buffer pH7.4 was added into target plate, and 20 ul/well of culture broth was transferred from expression plate to target plate. The plates were incubated at room temperature for 1 hour, and were then washed with PBST. 200 ul of BLA substrate nitrocefin in 50 mM PBS buffer pH6.5 were added into each well, and the bound ME66scFv was measured by monitoring hydrolysis of nitrocefin at wavelength 490 nm. The plate was left incubated in substrate buffer for an additional 1.5 hour. After quick rinsing with PBST, the bound ME66scFv-BLA was again measured using nitrocefin. The dissociation of ME66scFv from p97 was again measured between 3-6 hours after the initial time point and a binding index was calculated at 2 time points. In parallel, the plate was screened under identical conditions but using 50 mM PBS buffer at pH 7.4. The normalized screening results measured at pH 6.5 and at pH 7.4 are shown in the FIG. 5.

Table 6, below, shows mutations that have been observed in the three best variants.

TABLE 4
Mutations in affinity matured variants

| Clone | mutation |
|---|---|
| ME70.1 | heavy chain, S65K |
| ME70.7 | heavy chain, S65P |
| ME81.3 | heavy chain, N60R |

### EXAMPLE 3

### Stabilization of an scFv

A. Construction of pME27.1

Plasmid pME27.1 was generated by inserting a Bgl I EcoRV fragment encoding a part of the pelB leader, the CAB1-scFv and a small part of BLA into plasmid the expression vector pME25. The insert, encoding for the CAB1-scFv, has been synthesized by Aptagen (Herndon, Va.) based on the sequence of the scFv FE-23 that was described in [Boehm, M. K., A. L. Corper, T. Wan, M. K. Sohi, B. J. Sutton, J. D. Thornton, P. A. Keep, K. A. Chester, R. H. Begent and S. J. Perkins (2000) Biochem J 346 Pt 2, 519-28, Crystal structure of the anti-(carcinoembryonic antigen) single-chain Fv antibody MFE-23 and a model for antigen binding based on intermolecular contacts]. Both the plasmid containing the synthetic gene (pPCR-GME1) and pME25 were digested with BglI and EcoRV, gel purified and ligated together with Takara ligase. Ligation was transformed into TOP10(Invitrogen, Carlsbad, Calif.) electrocompetent cells, plated on LA medium containing 5 mg/l chloramphenicol and 0.1 mg/l cefotaxime.

Plasmid pME27.1 contains the following features:

P lac: 4992-5113 bp

pel B leader: 13-78

CAB 1 scFv: 79-810

BLA: 811-1896

T7 term.: 2076-2122

CAT: 3253-3912

A schematic of plasmid pME27.1 can be found in FIG. 6A. The CAB1 sequence, indicating heavy and light chain domains, can be found in FIG. 6B; the amino acid sequence can also be found in FIG. 6D, with linker and BLA.

B. Choosing Mutations for Mutagenesis

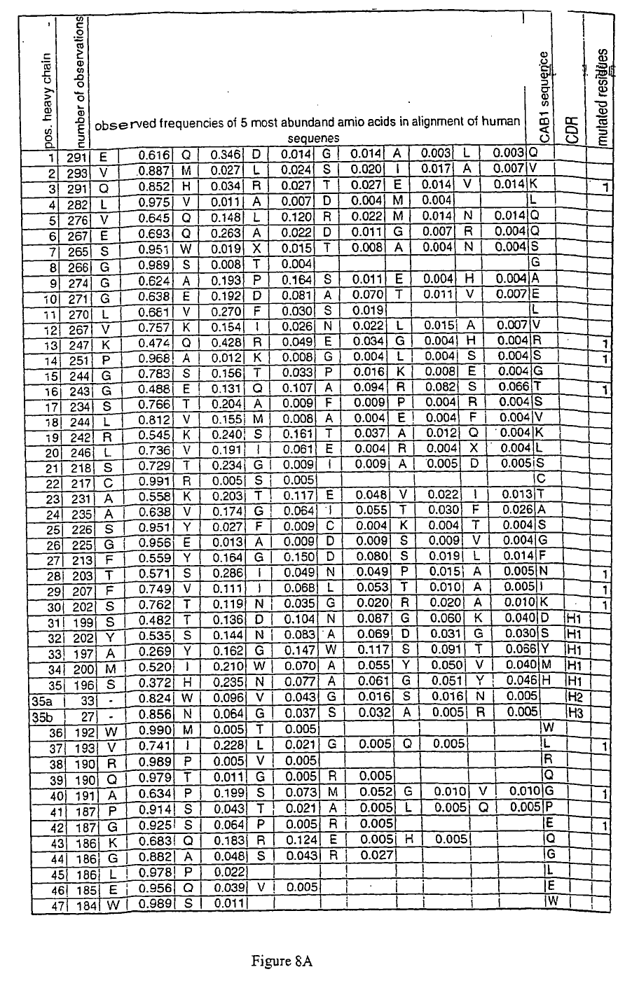

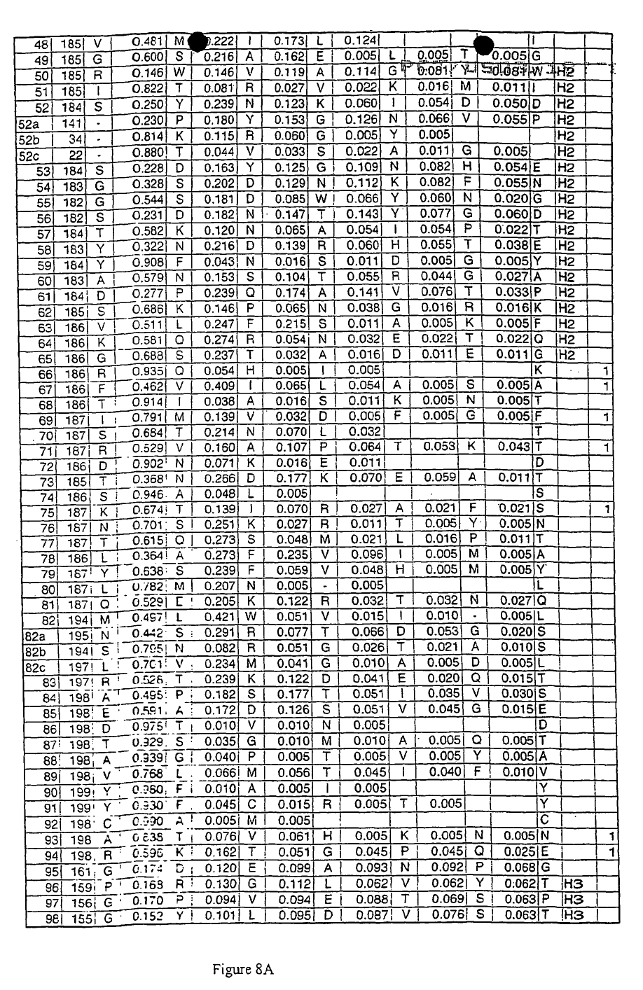

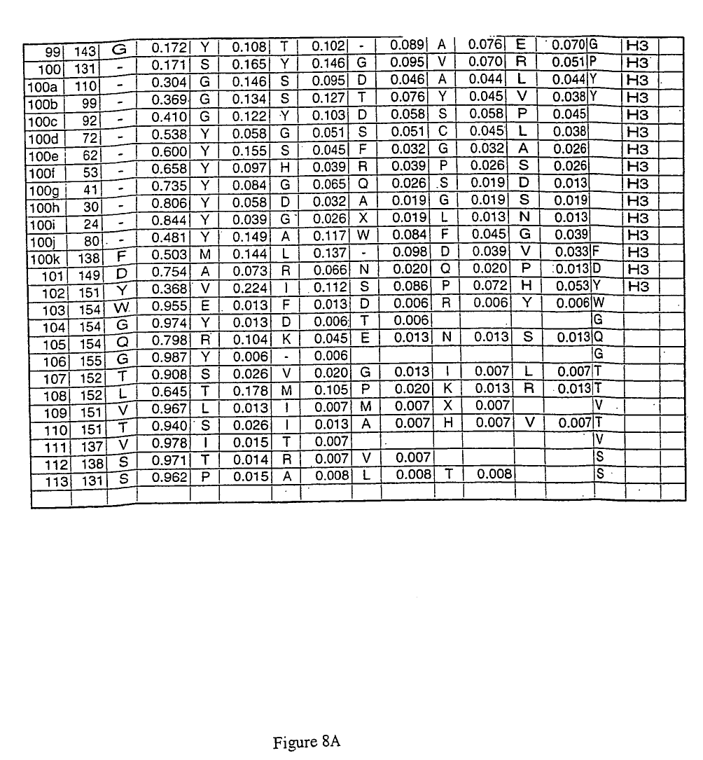

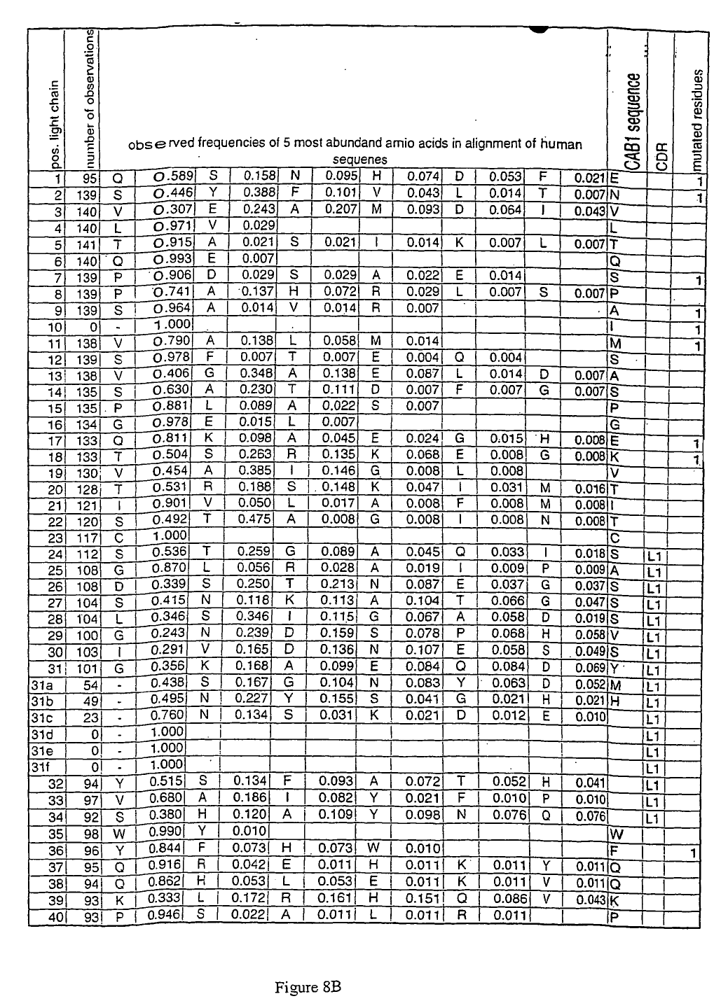

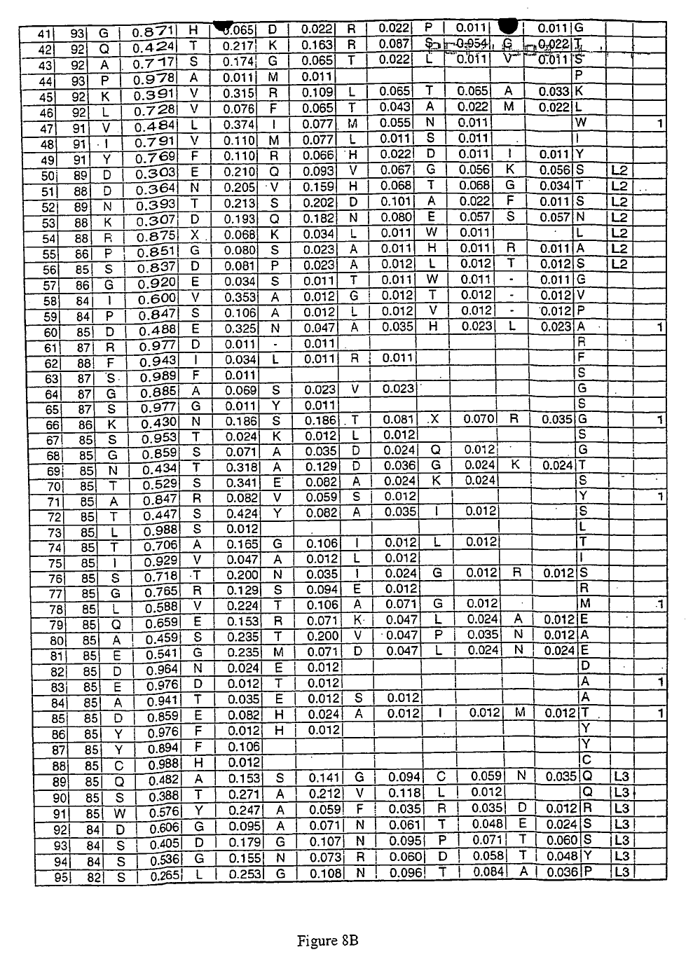

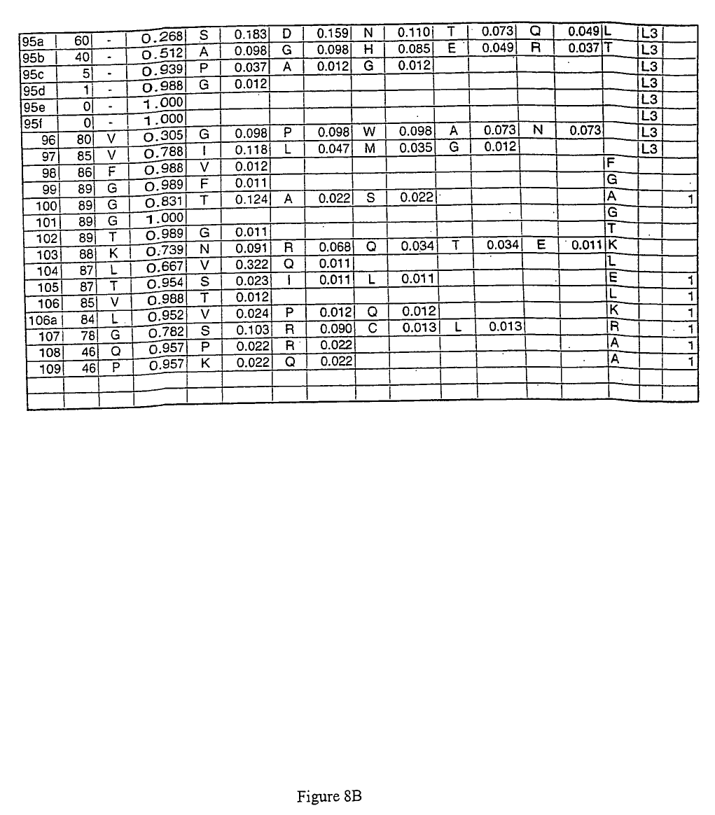

The sequence of the vH and vL sequences of CAB1-scFv were compared with a published frequency analysis of human antibodies (Boris Steipe (1998) Sequenzdatenanalyse. (“Sequence Data Analysis”, available in German only) in Bioanalytik eds. H. Zorbas und F. Lottspeich, Spektrum Akademischer Verlag. S. 233-241). The authors aligned sequences of variable segments of human antibodies as found in the Kabat data base and calculated the frequency of occurrence of each amino acid for each position. These alignment can be seen in FIG. 8. Specifically, FIG. 8A shows an alignment of the observed frequencies of the five most abundant amino acids in alignment of human sequences in the heavy chain. FIG. 8B shows an alignment of the observed frequencies of the five most abundant amino acids in alignment of human sequences in the light chain.

We compared these frequencies with the actual amino acid sequence of CAB1 and identified 33 positions that fulfilled the following criteria:
The position is not part of a CDR as defined by the Kabat nomenclature.
The amino acid found in CAB1-scFv is observed in the homologous position in less than 10% of human antibodies
The position is not one of the last 6 amino acids in the light chain of scFv.
   The resulting 33 positions were chosen for combinatorial mutagenesis.

Mutagenic oligonucleotides were synthesized for each of the 33 positions such that the targeted position would be changed from the amino acid in CAB1-scFv to the most abundant amino acid in the homologous position of a human antibody. FIG. 6B shows the sequence of CAB1-scFv, the CDRs, and the mutations that were chosen for combinatorial mutagenesis.

C. Constriction of Library NA05

Table 5 listing the sequences of 33 mutagenic oligonucleotides that were used to generate combinatorial library NA05:

TABLE 5

| pos. | | count | |
|---|---|---|---|
| (pME27) | | residues to | |
| MFE-23 | (VH) | be changed | QuikChange multi primer |
| 3K | Q | nsa147.1fp | CGGCCATGGCCCAGGTGCAGCTGCAGCAGTCTGGGGC |
| 13R | K | nsa147.2fp | CTGGGGCAGAACTTGTGAAATCAGGGACCTCAGTCAA |
| 14S | P | nsa147.3fp | GGGCAGAACTTGTGAGGCCGGGGACCTCAGTCAAGTT |
| 16T | G | nsa147.4fp | AACTTGTGAGGTCAGGGGGCTCAGTCAAGTTGTCCTG |
| 28N | T | nsa147.5fp | GCACAGCTTCTGGCTTCACCATTAAAGACTCCTATAT |
| 29I | F | nsa147.6fp | CAGCTTCTGGCTTCAACTTTAAAGACTCCTATATGCA |
| 30K | S | nsa147.7fp | CTTCTGGCTTCAACATTAGCGACTCCTATATGCACTG |
| 37L | V | nsa147.8fp | ACTCCTATATGCACTGGGTGAGGCAGGGGCCTGAACA |
| 40G | A | nsa147.9fp | TGCACTGGTTGAGGCAGGCGCCTGAACAGGGCCTGGA |
| 42E | G | nsa147.10fp | GGTTGAGGCAGGGGCCTGGCCAGGGCCTGGAGTGGAT |
| 67K | R | nsa147.11fp | CCCCGAAGTTCCAGGGCCGTGCCACTTTTACTACAGA |
| 68A | F | nsa147.12fp | CGAAGTTCCAGGGCAAGTTCACTTTTACTACAGACAC |
| 70F | I | nsa147.13fp | TCCAGGGCAAGGCCACTATTACTACAGACACATCCTC |
| 72T | R | nsa147.14fp | GCAAGGCCACTTTTACTCGCGACACATCCTCCAACAC |
| 76S | K | nsa147.15fp | TTACTACAGACACATCCAAAAACACAGCCTAGCTGCA |
| 97N | A | nsa147.16fp | CTGCCGTCTATTATTGTGCGGAGGGGACTCCGACTGG |
| 98E | R | nsa147.17fp | CCGTCTATTATTGTAATCGCGGGACTCCGACTGGGCC |
| 136E | Q | nsa147.18fp | CTGGCGGTGGCGGATCACAGAATGTGCTCACCCAGTC |
| 137N | S | nsa147.19fp | GCGGTGGCGGATCAGAAAGCGTGCTCACCCAGTCTCC |
| 142S | P | nsa147.20fp | GAAAATGTGCTCACCCAGCCGCCAGCAATCATGTCTGC |
| 144A | S | nsa147.21fp | TGCTCACCCAGTCTCCAAGCATCATGTCTGCATCTCC |
| 146M | V | nsa147.22fp | CCCAGTCTCCAGCAATCGTGTCTGCATCTCCAGGGGA |
| 152E | Q | nsa147.23fp | TGTCTGCATCTCCAGGGCAGAAGGTCACCATAACCTG |
| 153K | T | nsa147.24fp | CTGCATCTCCAGGGGAGACCGTCACCATAACCTGCAG |
| 170F | Y | nsa147.25fp | TAAGTTACATGCACTGGTACCAGCAGAAGCCAGGCAC |
| 181W | V | nsa147.26fp | GCACTTCTCCCAAACTCGTGATTTATAGCACATCCAA |
| 194A | D | nsa147.27fp | TGGCTTCTGGAGTCCCTGATCGCTTCAGTGGCAGTGG |
| 200G | K | nsa147.28fp | CTCGCTTCAGTGGCAGTAAATCTGGGACCTCTTACTC |
| 205Y | A | nsa147.29fp | GTGGATCTGGGACCTCTGCGTCTCTCACAATCAGCCG |
| 212M | L | nsa147.30fp | CTCTCACAATCAGCCGACTGGAGGCTGAAGATGCTGC |
| 217A | E | nsa147.31fp | GAATGGAGGCTGAAGATGAAGCCACTTATTACTGCCA |
| 219T | D | nsa147.32fp | AGGCTGAAGATGCTGCCGATTATTACTGCCAGCAAAG |
| 234A | G | nsa147.33fp | ACCCACTCACGTTCGGTGGCGGCACCAAGCTGGAGCT |

The QuikChange multi site-directed mutagenesis kit (QCMS; Stratagene Catalog # 200514) was used to construct the combinatorial library NA05 using 33 mutagenic primers. The primers were designed so that they had 17 bases flanking each side of the codon of interest based on the template plasmid pME27.1. The codon of interest was changed to encode the appropriate consensus amino acid using an E.coli codon usage table. All primers were designed to anneal to the same strand of the template DNA (i.e., all were forward primers in this case). The QCMS reaction was carried out as described in the QCMS manual with the exception of the primer concentration used, which ecommends using 50 ng of each primer in the reaction whereas we used around 3 ng of each primer. Other primer amounts may be used. In particular, the reaction contained 50-100 ng template plasmid (pME27.1; 5178 bp), 1 μl of primers mix (10 μM stock of all primers combined containing 0.3 μM each primer), 1 μl dNTPs (QCMS kit), 2.5 μl 10× QCMS reaction buffer, 18.5 μl deoinized water, and 1 μl enzyme blend (QCMS kit), for a total volume of 25 μl. The thermocycling program was 1 cycle at 95° for 1 min., followed by 30 cycles of 95° C. for 1 min., 55° C. for 1 min., and 65° C. for 10 minutes. DpnI digestion was performed by adding 1 μl DpnI (provided in the QCMS kit) incubation at 37° C. for 2 hours, addition of another 1 μl DpnI, and incubation at 37° C. for an additional 2 hours. 1 μl of the reaction was transformed into 50 μl of TOP10 electrocompetent cells from Invitrogen. 250 μl of SOC was added after electroporation, followed by a 1 hr incubation with shaking at 37° C. Thereafter, 10-50 μl of the tranformation mix was plated on LA plates with 5 ppm chloramphenicol (CMP) or LA plates with 5 ppm CMP and 0.1 ppm of cefotaxime (CTX) for selection of active BLA clones. The active BLA clones from the CMP+CTX plates were used for screening, whereas the random library clones from the CMP plates were sequenced to assess the quality of the library.

16 randomly chosen clones were sequenced. The clones contained different combinations of 1 to 7 mutations.

D. Screen for Improved Expression

We found that when TOP10/pME27.1 is cultured in LB medium at 37 C then the concentration of intact fusion protein peaks after one day and most of the fusion protein is degraded by host proteases after 3 days of culture. Degradation seems to occur mainly in the scFv portion of the CAB1 fusion protein as the cultures contain significant amounts of free BLA after 3 days, which can be detected by Western blotting, or nitrocefin (Oxoid, N.Y.) activity assay. Thus we applied a screen to library NA05 that was able to detect variants of CAB1-scFv that would resist degradation by host proteases over 3 days of culture at 37 C.

Library NA05 was plated onto agar plates with LA medium containing 5 mg/l chloramphenicol and 0.1 mg/l cefotaxime (Sigma). 910 colonies were transferred into a total of 10 96-well plates containing 100 ul/well of LA medium containing 5 mg/l chloramphenicol and 0.1 mg/l cefotaxime. Four wells in each plate were inoculated with TOP10/pME27.1 as control and one well per plate was left as a blank. The plates were grown overnight at 37 C. The next day the cultures were used to inoculate fresh plates (production plates) containing 100 ul of the same medium using a transfer stamping tool and glycerol was added to the master plates which were stored at −70 C. The production plates were incubated in a humidified shaker at 37 C for 3 days. 100 ul of BPER (Pierce, Rockford, Ill.) per well was added to the production plate to release protein from the cells. The production plate was diluted 100-fold in PBST (PBS containing 0.125% Tween-20) and BLA activity was measured by transferring 20 ul diluted lysate into 180 ul of nitrocephin assay buffer (0.1 mg/ml nitrocephin in 50 mM PBS buffer containing 0.125% octylglucopyranoside (Sigma)) and the BLA activity was determined at 490 nm using a Spectramax plus plate reader (Molecular Devices. Sunnyvale, Calif.).

Binding to CEA (carcinoembryonic antigen, Biodesign Intl., Saco, Me.) was measured using the following procedure: 96-well plates were coated with 100 ul per well of 5 ug/ml of CEA in 50 mM carbonate buffer pH 9.6 overnight. The plates were washed with PBST and blocked for 1-2 hours with 300 ul of casein (Pierce, Rockford, Ill.). 100 ul of sample from the production plate diluted 100-1000 fold was added to the CEA coated plate and the plates were incubated for 2 h at room temperature. Subsequently, the plates were washed four times with PBST and 200 ul nitrocefin assay buffer was added, and the BLA activity was measured as described above.

The BLA activity that was determined by the CEA-binding assay and the total BLA activity found in the lysate plates were compared and variants were identified which showed high levels of total BLA activity and high levels of CEA-binding activities.

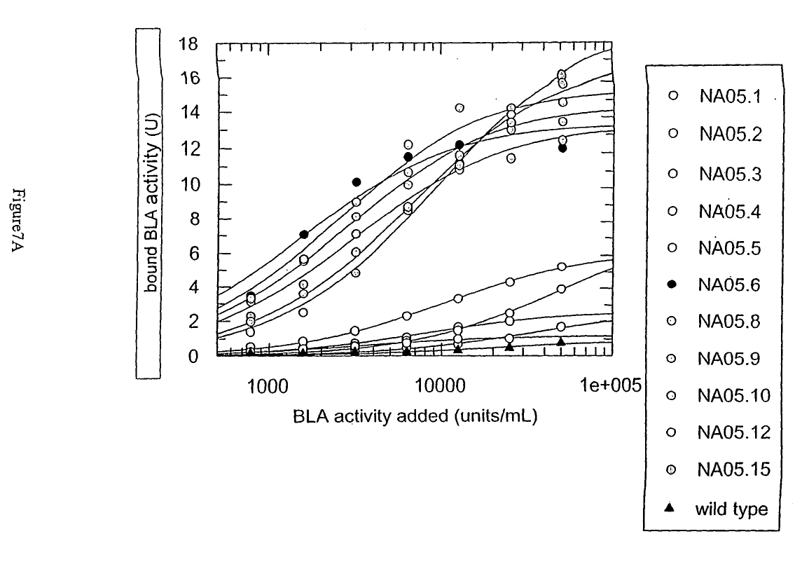

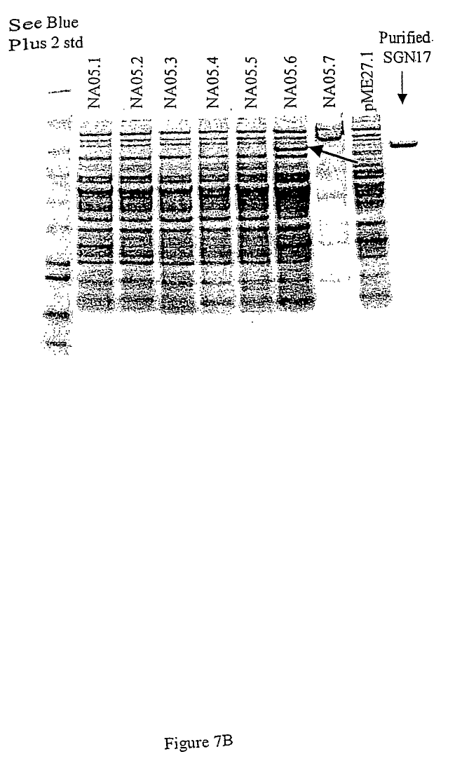

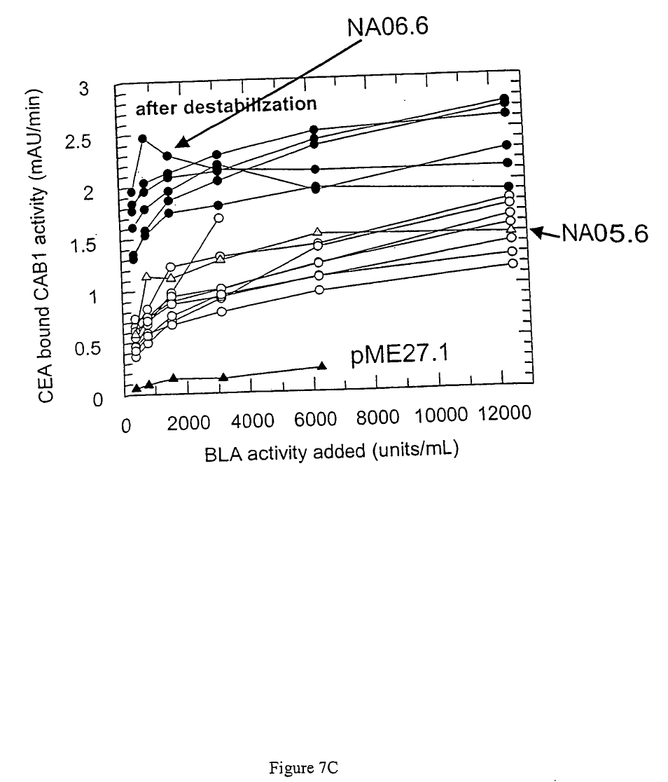

The winners were confirmed in 4 replicates using a similar protocol: the winners were cultured in 2 ml of LB containing 5 mg/l chloramphenicol and 0.1 mg/l cefotaxime for 3 days. Protein was released from the cells using BPER reagent. The binding assay was performed as described above but different dilutions of culture lysate were tested for each variant. Thus one can generate a binding curve which provides a measure of the binding affinity of the variant for the target CEA. FIG. 7A shows binding curves. Culture supernatants were also analyzed by SDS polyacrylamid electrophoresis. FIG. 7B shows the electropherogram of 7 variants from NA05. The band of the fusion protein is labeled for variant NA05.6. Table 6 shows a ranking of 6 variants. The data were normalized and a performance index was calculated as described in Example 1. The data clearly show that NA05.6 produces significantly larger quantities of fusion protein compared to the fusion construct pME27.1. Table 6 showing the sequence of 6 variants with the largest improvement in stability:

| clone | mutations |
|---|---|
| NA05.6 | R13K, T16G, W181V |
| NA05.8 | R13K, F170Y, A234G |
| NA05.9 | K3Q, S14P, L37V, E42G, E136Q, M146V, |
| | W181V, A234G |
| NA05.10 | K3Q, L37V, P170Y, W181V |
| NA05.12 | K3Q, S14P, L37V, M146V |
| NA05.15 | M146V, F170Y, A194D |

E. Construction of Library NA06

Clone NA05.6 was chosen as the best variant and was used as the template for a second round of combinatorial mutagenesis. We used a subset of the same mutagenic primers that had been used to generate library NA05 to generate combinatorial variants with the following mutations: K3Q, L37V, E42G, E136Q, M146V, F170Y, A194D, A234G which had been identified in other winners from library NA05. We did not use the primer encoding mutation S14P as its sequence overlapped with mutations R13K and T16G that are present in NA05.6. A combinatorial library was constructed using QuikChange Multisite as described above and was called NA06. Template was pNA05.6 and 1 μl of primers mix (10 μM stock of all primers combined containing 1.25 μM each primer) were used.

F. Screening of Library NA06

The screen was performed as described above with the following modifications: 291 variants were screened on 3 96-well plates. 10 μl sample from the lysate plates was added to 180 μl of 10 μg/ml thermolysin (Sigma) in 50 mM imidazole buffer pH 7.0 containing 0.005% Tween-20 and 10 mM calcium chloride. This mixture was incubated for 1 h at 37 C to hydrolyze unstable variants of NA05.6. This protease-treated sample was used to perform the CEA-binding assay as described above.

Promising variants were cultured in 2 ml medium as described above and binding curves were obtained for samples after thermolysin treatments. FIG. 7C shows binding curves for selected clones. It can be seen that a number of variants retain much more binding activity after thermolysin incubation than the parent NA05.6. Table 7 showing 6 variants which are significantly more protease resistant than NA05.6:

| Clone | mutations |
|---|---|
| NA06.2 | R13K, T16G, W181V, L37V, E42G, A194D |
| NA06.4 | R13K, T16G, W181V, L37V, M146V |
| NA06.6 | R13K, T16G, W181V, L37V, M146V, K3Q |
| NA06.10 | R13K, T16G, W181V, L37V, M146V, |
| | A194D |
| NA06.11 | R13K, T16G, W181V, L37V, K3Q, A194D |
| NA06.12 | R13K, T16G, W181V, L37V, E136Q |

All 6 variants have the mutation L37V which was rare in randomly chosen clones from the same library. Further testing showed that variant NA06.6 had the highest level of total BLA activity and the highest protease resistance of all variants.

### EXAMPLE 4

### Generation of an scFV that has pH-Dependent Binding

G. Choosing Positions for Mutagenesis

The 3D structure of the scFv portion of NA06.6 was modeled based on the published crystal structure of a close homologue, MFE-23 [Boehm, M. K., A. L. Corper, T. Wan, M. K. Sohi, B. J. Sutton, J. D. Thornton, P. A. Keep, K. A. Chester, R. H. Begent and S. J. Perkins (2000) Biochem J 346 Pt 2, 519-28, Crystal structure of the anti-(carcinoembryonic antigen) single-chain Fv antibody MFE-23 and a model for antigen binding based on intermolecular contacts] using the software pack-age MOE (Chemical Computing Group, Montreal, Canada) and using default parameters. A space filling model of the structure was visually inspected. Side chains in the CDRs were ranked as follows: 0=burried; 1=partially exposed; 2=completely exposed. Side chain distance to CDR3 was ranked as: 0=side chain is in CDR3; 1=side chain is one amino acid away from CDR3; 2=side chain is two amino acids away from CDR3. In a few cases, residues flanking the CDRs were included if they fit the distance and exposure criteria.

Based on this ranking, the following side chains were targeted for mutagenesis:

a) exposure=2 and distance=2 or smaller

b) exposure=1 and distance<2 40 positions in the CDRs matched these criteria.

FIG. 10 shows the CDRs and the residues that were chosen for mutagenesis.

The table below shows the criteria and position of the 40 sites that were chosen for mutagenesis.

H. Construction of Library NA08

A combinatorial library was constructed where the 40 selected positions were randomly replaced with aspartate or histidine. The substitutions were chosen as it has been reported that ionic interactions between histidine side chains and carboxyl groups form the structural basis for the pH-dependence of the interaction between IgG molecules and the Fc receptor [Vaughn, D. E. and P. J. Bjorkman (1998) Structure 6, 63-73., Structural basis of pH-dependent antibody binding by the neonatal Fc receptor].

The QuikChange multi site-directed mutagenesis kit (QCMS; Stratagene Catalog # 200514) was used to construct the combinatorial library NA08 using 40 mutagenic primers. The primers were designed so that they had 17 bases flanking each side of the codon of interest based on the template plasmid NA06.6. The codon of interest was changed to the degenerate codon SAT to encode for aspartate and histidine. All primers were designed to anneal to the same strand of the template DNA (i.e., all were forward primers in this case). The QCMS reaction was carried out as described in the QCMS manual with the exception of the primer concentration used; the manual recommends using 50-100 ng of each primer in the reaction, whereas significantly lower amounts of each primer were used in this library as this results in a lower parent template background. In particular, 0.4 μM of all primers together were used. The individual degenerate primer concentration in the final reaction was 0.01 μM (approximately 2.5 ng).

The QCMS reaction contained 50-100 ng template plasmid (NA06.6, 5178 bp), 1 μl of primers mix (10 μM stock of all primers to give the desired primer concentration mentioned above), 1 μl dNTPs (QCMS kit), 2.5 μl 10× QCMS reaction buffer, 18.5 μl deoinized water, and 1 μl enzyme blend (QCMS kit), for a total volume of 25 μl. The thermocycling program was 1 cycle at 95° for 1 min., followed by 30 cycles of 95° C. for 1 min., 55° C. for 1 min., and 65° C. for 10 minutes. Dpnl digestion was performed by adding 1 μl DpnI (provided in the QCMS kit), incubation at 37° C. for 2 hours, addition of 0.5 μl DpnI, and incubation at 37° C. for an additional 2 hours. 1 μl of each reaction was transformed into 50 μl of TOP10 electrocompetent cells from Invitrogen. 250 μl of SOC was added after electroporation, followed by a 1 hr incubation with shaking at 37° C. Thereafter, 10-50 μl of the transformation mix was plated on LA plates with 5 ppm chloramphenicol (CMN) or LA plates with 5 ppm CMP and 0.1 ppm of cefotaxime (CTX) for selection of active BLA clones. The number of colonies obtained on both types of plates was comparable (652 on the CMP plate and 596 colonies on the CMP+CTX plate for 10 μl of the transformation mix plated). Active BLA clones from the CMP+CTX plates were used for screening, whereas random library clones from the CMP plates were sequenced to assess the quality of the library.

Primers for the reaction are shown in Table 8.

TABLE 8
Primers for CDRs:

| | | | | |
|---|---|---|---|---|
| | | position | distance | |
| residue | CDRs | exposure | to CDR3 | primer sequence |

| | | | | |
|---|---|---|---|---|
| K | | 30 | 2 | 2 cttctggcttcaacattsatgactcctatatgcactg |
| D | H1 | 31 | 2 | 1 ctggcttcaacattaaasattcctatatgcactgggt |
| S | H1 | 32 | 1 | 1 gcttcaacattaaagacsattatatgcactgggtgag |
| Y | H1 | 33 | 2 | 1 tcaacattaaagactccsatatgcactgggtgaggca |
| H | H1 | 35 | 1 | 1 ttaaagactcctatatgsattgggtgaggcaggggcc |
| W | H2 | 50 | 2 | 1 gcctggagtggattggasatattgatcctgagaatgg |
| D | H2 | 52 | 2 | 2 agtggattggatggattsatcctgagaatggtgatac |
| E | H2 | 54 | 2 | 2 ttggatggattgatcctsataatggtgatactgaata |
| N | H2 | 55 | 2 | 2 gatggattgatcctgagsatggtgatactgaatatgc |
| D | H2 | 57 | 2 | 1 ttgatcctgagaatggtsatactgaatatgccccgaa |
| T | H2 | 58 | 1 | 1 atcctgagaatggtgatsatgaatatgccccgaagtt |
| E | H2 | 59 | 2 | 1 ctgagaatggtgatactsattatgccccgaagttcca |
| P | H2 | 62 | 2 | 1 gtgatactgaatatgccsataagttccagggcaaggc |
| K | H2 | 63 | 2 | 3 atactgaatatgccccgsatttccagggcaaggccac |
| Q | H2 | 65 | 2 | 2 aatatgccccgaagttcsatggcaaggccacttttac |
| E | | 98 | 1 | 0 ccgtctattattgtaatsatgggactccgactgggcc |
| G | | 99 | 1 | 0 tctattattgtaatgagsatactccgactgggccgta |
| T | H3 | 100 | 2 | 0 attattgtaatgaggggsatccgactgggccgtacta |
| P | H3 | 101 | 2 | 0 attgtaatgaggggactsatactgggccgtactactt |
| T | H3 | 102 | 2 | 0 gtaatgaggggactccgsatgggccgtactactttga |
| G | H3 | 103 | 2 | 0 atgaggggactccgactsatccgtactactttgacta |
| P | H3 | 104 | 2 | 0 aggggactccgactgggsattactactttgactactg |
| Y | H3 | 106 | 2 | 0 ctccgactgggccgtacsattttgactactggggcca |
| S | L1 | 162 | 2 | 2 taacctgcagtgccagcsatagtgtaagttacatgca |
| S | L1 | 163 | 2 | 1 cctgcagtgccagctcasatgtaagttacatgcactg |
| V | L1 | 164 | 1 | 1 gcagtgccagctcaagtsatagttacatgcactggtt |
| S | L1 | 165 | 2 | 1 gtgccagctcaagtgtasattacatgcactggttcca |
| Y | L1 | 166 | 2 | 1 ccagctcaagtgtaagtsatatgcactggttccagca |
| Y | | 183 | 1 | 0 ctcccaaactcgtgattsatagcacatccaacctggc |
| S | L2 | 184 | 2 | 0 ccaaactcgtgatttatsatacatccaacctggcttc |
| T | L2 | 185 | 1 | 1 aactcgtgatttatagcsattccaacctggcttctgg |
| S | L2 | 186 | 2 | 2 tcgtgatttatagcacasataacctggcttctggagt |
| N | L2 | 187 | 2 | 1 tgatttatagcacatccsatctggcttctggagtccc |
| A | L2 | 189 | 1 | 1 atagcacatccaacctgsattctggagtccctgctcg |
| S | L2 | 190 | 2 | 1 gcacatccaacctggctsatggagtccctgctcgctt |
| R | L3 | 225 | 2 | 2 cttattactgccagcaasattctagttacccactcac |
| S | L3 | 226 | 2 | 2 attactgccagcaaagasatagttacccactcacgt |
| S | L3 | 227 | 1 | 2 actgccagcaaagatctsattacccactcacgttcg |
| Y | L3 | 228 | 1 | 2 gccagcaaagatctagtsatccactcacgttcggtg |
| L | L3 | 230 | 1 | 2 aaagatctagttacccasatacgttcggtgctggcac |

I. Sequencing of Variants

Variants were grown overnight with shaking at 37° C. in 5 mL cultures of LA containing 5 ppm of CMP. Miniprep DNA was prepared using a Qiagen kit and the BLA gene within each clone was sequenced using the M13 reverse and nsa154f primers.

M13 reverse: CAGGAAACAGCTATGAC

nsa154f: GGACCACGGTCACCGTCTCCTC

J. Screen pH-Dependent Binding

Library NA08 was plated onto agar plates with LA medium containing 5 mg/l chloramphenicol and 0.1 mg/l cefotaxime (Sigma). 552 colonies were transferred into a total of six 96-well plates containing 100 ul/well of LA medium containing 5 mg/l chloramphenicol and 0.1 mg/l cefotaxime. Four wells in each plate were inoculated with TOP10/NA06.6 as a reference. The plates were grown overnight at 37 C. The next day the cultures were used to inoculate fresh plates (production plates) containing 100 ul of the same medium using a transfer stamping tool and glycerol was added to the master plates which were stored at −70 C. The production plates were incubated in a humidified shaker at 37 C for 2 days. 100 ul of BPER (Pierce, Rockford, Ill.) per well was added to the production plates to release protein from the cells. The production plates were diluted 100-fold in PBST (PBS containing 0.125% Tween-20) and BLA activity was measured by transferring 20 ul diluted lysate into 170 ul of nitrocefin assay buffer (0.1 mg/ml nitrocefin in 50 mM PBS buffer containing 0.125% octylglucopyranoside (Sigma)) and the BLA activity was determined at 490 nm using a Spectramax plus plate reader (Molecular Devices, Sunnyvale, Calif.).

Binding to CEA (carcinoembryonic antigen, Biodesign Intl., Saco, Me.) was measured using the following procedure: 96-well plates were coated with 100 ul per well of 5 ug/ml of CEA in 50 mM carbonate buffer pH 9.6 overnight. The plates were washed with PBST and blocked for 1-2 hours with 300 ul of casein (Pierce, Rockford, Ill.). 100 ul of sample from the production plate diluted 100-1000 fold was added to the CEA coated plate and the plates were incubated for 2 h at room temperature. Subsequently, the plates were washed four times with PBST and 200 ul nitrocefin assay buffer was added, and the BLA activity was measured as described above. CEA binding was measured in 50 mM phosphate buffer pH 6.5 and in a separate experiment in 50 mM phosphate buffer pH 7.4.

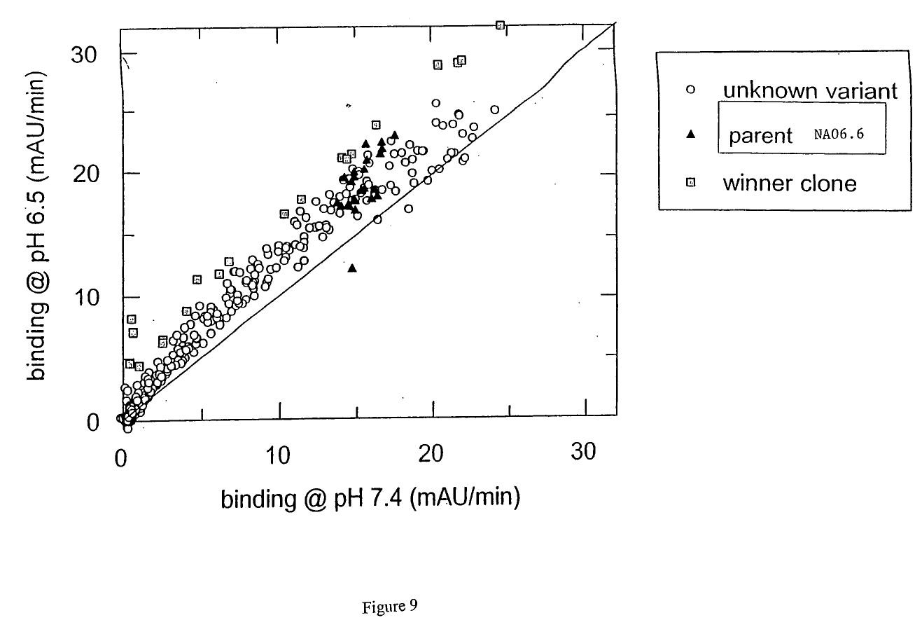

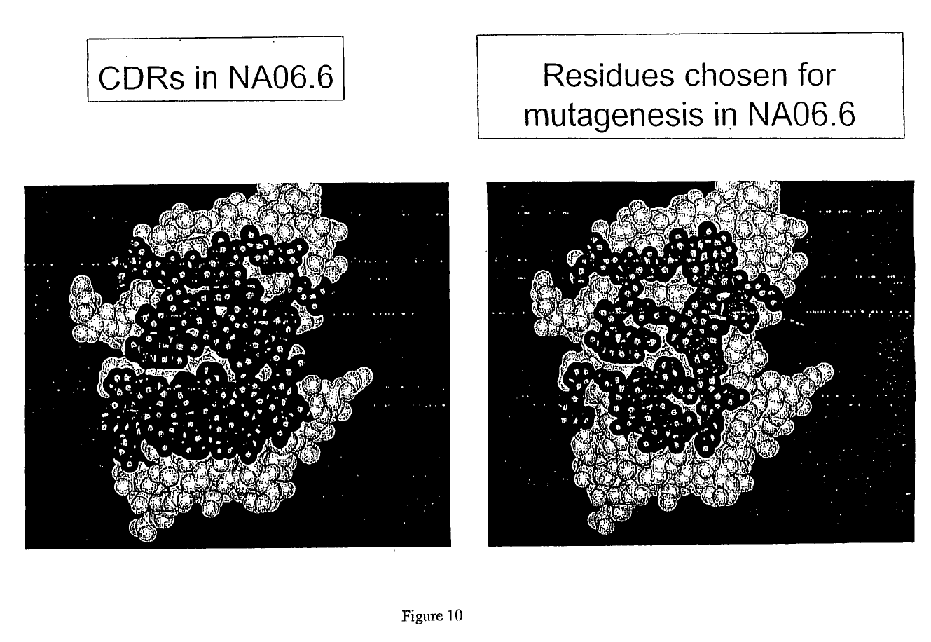

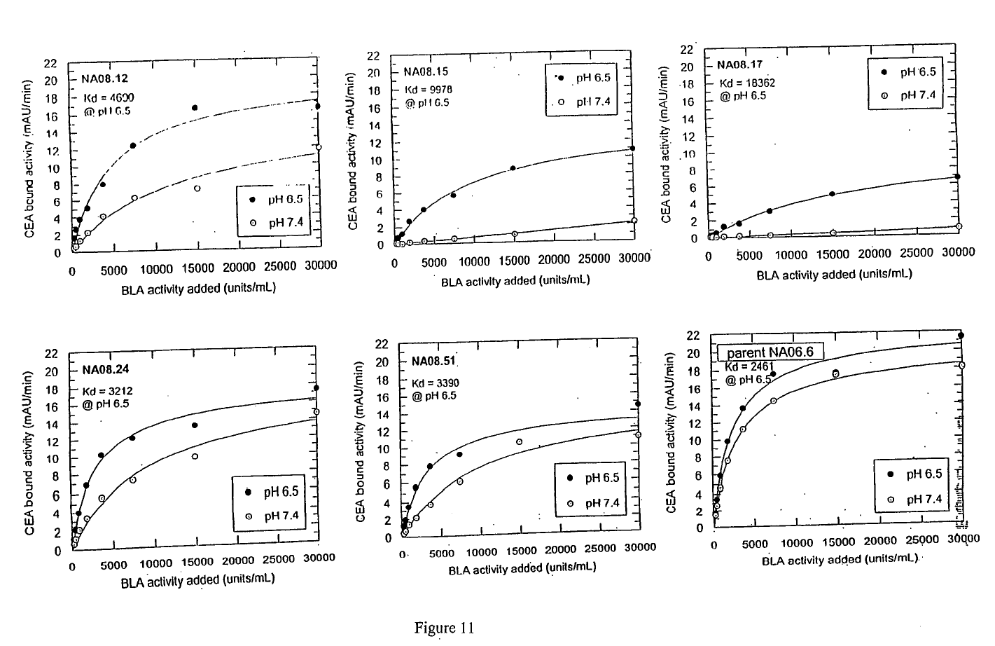

The BLA activity that was determined by the CEA-binding assay at pHs of 6.5 and 7.4, and the total BLA activity found in the lysate plates were compared and variants were identified which showed good binding to CEA at pH 6.5 but significantly weaker binding at pH 6.5. A comparison of the binding at pH6.5 versus pH 7.4 is shown in FIG. 9.

Winners were confirmed by culturing them in 5 ml of LB medium containing 5 mg/l chloramphenicol and 0.1 mg/l cefotaxime (Sigma) for 2 days at 37 C. Subsequently, the cultures were centrifuged and the pellet was suspended in 375 ul of BPER reagent to release the fusion protein. The BLA activity in each sample was determined by transferring 20 ul of the appropriately diluted sample to 180 ul of 180 ul of nitrocefin assay buffer (0.1 mg/ml nitrocefin in 50 mM PBS buffer containing 0.125% octylglucopyranoside (Sigma)) and the absorbance at 490 nm was monitored. One unit of activity was defined as the amount of BLA that leads to an absorbance increase of one mOD per minute. The samples were diluted based on their total content of BLA activity and the CEA-binding assay was performed as described above but adding various sample dilutions to each well.

Thus, one can obtain binding curves for each sample that reflect the affinity of the variants to CEA. FIG. 1 shows CEA-binding curves measured at pH 7.4 and pH 6.5 for several variants of interest. All 5 variants show increased pH-dependence of CEA binding. Whereas, the parent NA06.6 binds only slightly better at pH 6.5 compared to pH 7.4, some of the variant show much stronger binding to CEA at pH 6.5 compared to pH 7.4. Of particular interest are variants NA08. 15 and NA08.17 which show very weak binding to CEA at pH 7.4 but significant binding at pH 6.5.

Table 9, below, shows variants with the greatest binding improvement.

TABLE 9

| clone | mutations |
|---|---|
| NA08.1 | W50H, Y166D |
| NA08.3 | S190D, S226D |
| NA08.4 | S190D, T100D |
| NA08.9 | Y166D |
| NA08.12 | T102H, Y166D, S226D |
| NA08.13 | Q65H, S184D, S226D |
| NA08.14 | P101D |
| NA08.15 | S184D, S226D |
| NA08.17 | S184D, W50H |
| NA08.24 | T102D, S226D |
| NA08.45 | T102D, Y166D |
| NA08.51 | P104H, Y166D |
| NA08.64 | Q65D, Y166D |

### EXAMPLE 5

### In Vivo Tumor Model Assay

An assay could be constructed measuring tumor/normal binding based upon pH. Tumors compartments are normally more acidic. In vivo assays could be carried out using in vivo tumor and normal models expressing a target of interest. An MDTA with an increased binding or affinity or decreased dissociation rate at a pH lower than normally observed in normoxic non-tumorous normal tissues should result in greater binding and/or retention of the MDTA in the tumor compartment versus the normal compartments resulting in a greater efficacy by the MDTA for a desired effect.

What is claimed is:

1. A method of identifying a milieu-dependent targeted agent (MDTA), comprising contacting a microtarget in a first milieu and the microtarget in a second milieu with an agent, wherein the agent is a MDTA if it preferentially binds the microtarget in the first milieu over binding the microtarget in the second milieu.

2. The method of claim 1 wherein the first milieu is a physiological condition associated with a disease state and the second milieu is a physiological condition associated with a healthy or normal state.

3. The method of claim 2 wherein the disease state is cancer.

4. The method of claim 1 wherein the first milieu has a lower pH than the second milieu.

5. The method of claim 4 wherein the contacting occurs in the interstitial space of a tissue.

6. The method of claim 1 wherein all or part of p97, CEA, MUC-1, ED-B or TAG-72 comprises the microtarget.

7. The method of claim 6, wherein the target is CEA and the contacting occurs in the interstitial space of a tissue.

8. A method of increasing the milieu-dependent binding of an agent, comprising
a) contacting a microtarget in a first milieu with a modified form of the agent, wherein an unmodified form of the agent preferentially binds the microtarget in the first milieu relative to binding the microtarget in a second milieu, and
b) selecting the modified form of the agent if its preference for binding the microtarget in the first milieu relative to its binding of the microtarget in the second milieu is greater than the preference of the unmodifed form of the agent for binding the microtarget in the first milieu relative to its binding the microtarget in the second milieu,
wherein the selected agent is a milieu-dependent targeted agent (MDTA).

9. The method of claim 6 further comprising repeating steps (a) and (b) one or more times, wherein an agent selected in a previous step (b) is the unmodified form of the agent of step (a).

10. The method of claim 6 wherein the first milieu is a physiological condition associated with a disease state and the second milieu is a physiological condition associated with a healthy or normal state.

11. The method of claim 10 wherein the disease state is cancer.

12. The method of claim 8 wherein the first milieu has a lower pH than the second milieu.

13. The method of claim 12, wherein the contacting occurs in the interstitial space of a tissue.

14. The method of claim 6 wherein all or part of p97, CEA, MUC-1, ED-B or TAG-72 comprises the microtarget.

15. The method of claim 14, wherein the contacting occurs in the interstitial space of a tissue and the target is CEA.

16. A method of preferentially binding a milieu-dependent targeted agent (MDTA) to a target comprising contacting the target and a non-target with the MDTA, wherein the target comprises a microtarget in a first milieu, the nontarget comprises the microtarget in a second milieu, and the first milieu is not identical to the second milieu, under conditions that allow the MDTA to preferentially bind the microtarget in the first milieu over binding the microtarget in the second milieu.

17. The method of claim 16 wherein the MDTA binds the microtarget in the target but not the microtarget in the non-target.

18. The method of claim 16 wherein the first milieu is a physiological condition associated with a disease state and the second milieu is a physiological condition associated with a healthy or normal state.

19. The method of claim 18 wherein the disease state is cancer.

20. The method of claim 16 wherein the first milieu has a lower pH than the second milieu.

21. The method of claim 16 wherein all or part of p97, CEA, MUC-1, ED-B or TAG-72 comprises the microtarget.

22. The method of claim 16 wherein the MDTA is administered to a subject comprising the target and the non-target.

23. The method of claim 22 wherein the target is a cancerous cell, tissue or organ.

24. The method of claim 23 wherein all or part of p97, CEA, MUC-1, ED-B or TAG-72 comprises the microtarget.

25. A method of binding a MDTA to at least one microtarget in a compartment, comprising manipulating the compartment and contacting the at least one microtarget with the MDTA under conditions that allow the at least one microtarget to bind the MDTA.

26. A method of detecting a diseased, injured or infected tissue from a subject comprising
manipulating a compartment having the tissue
contacting the tissue from the subject with a detectable MDTA that preferentially binds the diseased, injured or infected tissue over a healthy tissue, and
detecting the binding of the detectable MDTA to the tissue from the subject,
wherein an increase in binding of the detectable MDTA to the tissue from the subject relative to the binding of the MDTA to healthy tissue indicates that the subject has a diseased, injured or infected tissue.

27. A method of identifying a MDTA comprising
a) manipulating a compartment, the compartment containing at least one microtarget and having a first milieu, the manipulation creating a second milieu in the compartment, the second milieu being different from the first milieu
b) contacting the at least one microtarget in the first milieu with a modified form of an agent, wherein an unmodified form of the agent binds the at least one microtarget in the first milieu and the microtarget in the second milieu about equally, and
c) selecting the modified form of the agent if it preferentially binds the at least one microtarget in the first milieu relative to its binding of the at least one microtarget in the second milieu,
wherein the selected modified form of the agent is the MDTA.

28. A MDTA, comprising a binding moiety that preferentially binds to a microtarget present on a target relative to binding of the microtarget present on a non-target.

29. The MDTA according to claim 28, wherein the preferential binding is effected as a result of a difference in pH.

30. The MDTA according to claim 29, wherein the difference between pH is between 6.5 and 7.4.

31. The MDTA according to claim 29 or claim 30, wherein said MDTA is targeted at CEA, TAG-72, MUC-1, ED-B or p97.

32. The MDTA according to claim 31, wherein said MDTA is targeted at CEA.

33. The MDTA according to claim 32, wherein the MDTA has a sequence as set forth in FIG. 1 or FIG. 6.


SEQUENCE LISTING (263 sequences)

SEQ ID NO: 1 - protein, 361 aa, Artificial Sequence
  beta-lactamase
TPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 2 - protein, 605 aa, Artificial Sequence
  CAB1 scFv
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAATPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 3 - DNA, 5178 bp, Artificial Sequence
  pME27.1 plasmid
aggaattatcatatgaaatacctgctgccgaccgctgctgctggtctgctgctcctcgctgcccagccggccatggcccaggtgaaactgcagcagtctggggcagaacttgtgaggtcagggacctcagtcaagttgtcctgcacagcttctggcttcaacattaaagactcctatatgcactggttgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggactccgactgggccgtactactttgactactggggccaagggaccacggtcaccgtctcctcaggtggaggcggttcaggcggaggtggctctggcggtggcggatcagaaaatgtgctcacccagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaagatctagttacccactcacgttcggtgctggcaccaagctggagctgaaacgggcggccacaccggtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacagtaggaattcgagctccgtcgacaagcttgcggccgcactcgagatcaaacgggctagccagccagaactcgccccggaagaccccgaggatgtcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgtggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcacgatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctttctagattaagg

SEQ ID NO: 4 - protein, 120 aa, Artificial Sequence
  CAB1 heavy chain
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSS

SEQ ID NO: 5 - protein, 15 aa, Artificial Sequence
  CAB1 linker
GGGGSGGGGSGGGGS

SEQ ID NO: 6 - protein, 110 aa, Artificial Sequence
  CAB1 light chain
ENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAAT

SEQ ID NO: 7 - protein, 360 aa, Artificial Sequence
  beta-lactamase
PVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 8 - protein, 8 aa, Artificial Sequence
  beta-lactamase
RLYANASI

SEQ ID NO: 9 - protein, 4 aa, Artificial Sequence
  beta-lactamase
KTXS

SEQ ID NO: 10 - protein, 8 aa, Artificial Sequence
  beta-lactamase
VHKTGSTG

SEQ ID NO: 11 - DNA, 37 bp, Artificial Sequence
  primer
actacaatccatctctccatagtcgcatttccatcac

SEQ ID NO: 12 - DNA, 37 bp, Artificial Sequence
  primer
gtgatggaaatgcgactatggagagatggattgtagt

SEQ ID NO: 13 - DNA, 39 bp, Artificial Sequence
  primer
gccacatattactgtgcacataggactctggctacttac

SEQ ID NO: 14 - DNA, 39 bp, Artificial Sequence
  primer
gtaagtagccagagtcctatgtgcacagtaatatgtggc

SEQ ID NO: 15 - DNA, 37 bp, Artificial Sequence
  primer
catattactgtgcaagacatactctggctacttacta

SEQ ID NO: 16 - DNA, 37 bp, Artificial Sequence
  primer
tagtaagtagccagagtatgtcttgcacagtaatatg

SEQ ID NO: 17 - DNA, 37 bp, Artificial Sequence
  primer
attactgtgcaagaaggcatctggctacttactatgc

SEQ ID NO: 18 - DNA, 37 bp, Artificial Sequence
  primer
gcatagtaagtagccagatgccttcttgcacagtaat

SEQ ID NO: 19 - DNA, 37 bp, Artificial Sequence
  primer
actgtgcaagaaggactcatgctacttactatgctat

SEQ ID NO: 20 - DNA, 37 bp, Artificial Sequence
  primer
atagcatagtaagtagcatgagtccttcttgcacagt

SEQ ID NO: 21 - DNA, 37 bp, Artificial Sequence
  primer
gtgcaagaaggactctgcatacttactatgctatgga

SEQ ID NO: 22 - DNA, 37 bp, Artificial Sequence
  primer
tccatagcatagtaagtatgcagagtccttcttgcac

SEQ ID NO: 23 - DNA, 37 bp, Artificial Sequence
  primer
caagaaggactctggctcattactatgctatggacta

SEQ ID NO: 24 - DNA, 37 bp, Artificial Sequence
  primer
tagtccatagcatagtaatgagccagagtccttcttg

SEQ ID NO: 25 - DNA, 37 bp, Artificial Sequence
  primer
gaaggactctggctactcattatgctatggactactg

SEQ ID NO: 26 - DNA, 37 bp, Artificial Sequence
  primer
cagtagtccatagcataatgagtagccagagtccttc

SEQ ID NO: 27 - DNA, 37 bp, Artificial Sequence
  primer
ggactctggctacttaccatgctatggactactgggg

SEQ ID NO: 28 - DNA, 37 bp, Artificial Sequence
  primer
ccccagtagtccatagcatggtaagtagccagagtcc

SEQ ID NO: 29 - DNA, 37 bp, Artificial Sequence
  primer
ctctggctacttactatcatatggactactggggtca

SEQ ID NO: 30 - DNA, 37 bp, Artificial Sequence
  primer
tgaccccagtagtccatatgatagtaagtagccagag

SEQ ID NO: 31 - DNA, 37 bp, Artificial Sequence
  primer
tggctacttactatgctcatgactactggggtcaagg

SEQ ID NO: 32 - DNA, 37 bp, Artificial Sequence
  primer
ccttgaccccagtagtcatgagcatagtaagtagcca

SEQ ID NO: 33 - DNA, 37 bp, Artificial Sequence
  primer
ctacttactatgctatgcattactggggtcaaggaac

SEQ ID NO: 34 - DNA, 37 bp, Artificial Sequence
  primer
gttccttgaccccagtaatgcatagcatagtaagtag

SEQ ID NO: 35 - DNA, 37 bp, Artificial Sequence
  primer
cttactatgctatggaccattggggtcaaggaacctc

SEQ ID NO: 36 - DNA, 37 bp, Artificial Sequence
  primer
gaggttccttgaccccaatggtccatagcatagtaag

SEQ ID NO: 37 - DNA, 37 bp, Artificial Sequence
  primer
actatgctatggactaccatggtcaaggaacctctgt

SEQ ID NO: 38 - DNA, 37 bp, Artificial Sequence
  primer
acagaggttccttgaccatggtagtccatagcatagt

SEQ ID NO: 39 - DNA, 37 bp, Artificial Sequence
  primer
caaagctcctgatctaccatgtttccaaccgattttc

SEQ ID NO: 40 - DNA, 37 bp, Artificial Sequence
  primer
gaaaatcggttggaaacatggtagatcaggagctttg

SEQ ID NO: 41 - DNA, 43 bp, Artificial Sequence
  primer
gattttctggggtcccagaccatttcagtggcagtggatcagg

SEQ ID NO: 42 - DNA, 43 bp, Artificial Sequence
  primer
cctgatccactgccactgaaatggtctgggaccccagaaaatc

SEQ ID NO: 43 - DNA, 37 bp, Artificial Sequence
  primer
gagtttatttctgctctcatagtacacatgttcctcc

SEQ ID NO: 44 - DNA, 37 bp, Artificial Sequence
  primer
ggaggaacatgtgtactatgagagcagaaataaactc

SEQ ID NO: 45 - DNA, 39 bp, Artificial Sequence
  primer
gtttatttctgctctcaacatacacatgttcctccgacg

SEQ ID NO: 46 - DNA, 39 bp, Artificial Sequence
  primer
cgtcggaggaacatgtgtatgttgagagcagaaataaac

SEQ ID NO: 47 - DNA, 45 bp, Artificial Sequence
  primer
gtttatttctgctctcaaagtcatcatgttcctccgacgttcggt

SEQ ID NO: 48 - DNA, 45 bp, Artificial Sequence
  primer
accgaacgtcggaggaacatgatgactttgagagcagaaataaac

SEQ ID NO: 49 - DNA, 37 bp, Artificial Sequence
  primer
tctgctctcaaagtacacatgttcctccgacgttcgg

SEQ ID NO: 50 - DNA, 37 bp, Artificial Sequence
  primer
ccgaacgtcggaggaacatgtgtactttgagagcaga

SEQ ID NO: 51 - DNA, 37 bp, Artificial Sequence
  primer
gctctcaaagtacacatcatcctccgacgttcggtgg

SEQ ID NO: 52 - DNA, 37 bp, Artificial Sequence
  primer
ccaccgaacgtcggaggatgatgtgtactttgagagc

SEQ ID NO: 53 - DNA, 37 bp, Artificial Sequence
  primer
ctcaaagtacacatgttcatccgacgttcggtggagg

SEQ ID NO: 54 - DNA, 37 bp, Artificial Sequence
  primer
cctccaccgaacgtcggatgaacatgtgtactttgag

SEQ ID NO: 55 - DNA, 39 bp, Artificial Sequence
  primer
caaagtacacatgttcctcatacgttcggtggaggcacc

SEQ ID NO: 56 - DNA, 39 bp, Artificial Sequence
  primer
ggtgcctccaccgaacgtatgaggaacatgtgtactttg

SEQ ID NO: 57 - DNA, 39 bp, Artificial Sequence
  primer
agtacacatgttcctccgcatttcggtggaggcaccaag

SEQ ID NO: 58 - DNA, 39 bp, Artificial Sequence
  primer
cttggtgcctccaccgaaatgcggaggaacatgtgtact

SEQ ID NO: 59 - DNA, 37 bp, Artificial Sequence
  primer
ctggcgactccatcaccnnsggttactggaactggat

SEQ ID NO: 60 - DNA, 37 bp, Artificial Sequence
  synthetic oligonucleotide
atccagttccagtaaccsnnggtgatggagtcgccag

SEQ ID NO: 61 - DNA, 37 bp, Artificial Sequence; identical to SEQ ID NO: 59
  synthetic oligonucleotide

SEQ ID NO: 62 - DNA, 37 bp, Artificial Sequence; identical to SEQ ID NO: 60
  synthetic oligonucleotide

SEQ ID NO: 63 - DNA, 37 bp, Artificial Sequence
  synthetic oligonucleotide
gcgactccatcaccagtnnstactggaactggatccg

SEQ ID NO: 64 - DNA, 37 bp, Artificial Sequence
  synthetic oligonucleotide
cggatccagttccagtasnnactggtgatggagtcgc

SEQ ID NO: 65 - DNA, 37 bp, Artificial Sequence
  synthetic oligonucleotide
actccatcaccagtggtnnstggaactggatccggca

SEQ ID NO: 66 - DNA, 37 bp, Artificial Sequence
  synthetic oligonucleotide
tgccggatccagttccasnnaccactggtgatggagt

SEQ ID NO: 67 - DNA, 39 bp, Artificial Sequence
  synthetic oligonucleotide
tccatcaccagtggttacnnsaactggatccggcagttc

SEQ ID NO: 68 - DNA, 39 bp, Artificial Sequence
  synthetic oligonucleotide
gaactgccggatccagttsnngtaaccactggtgatgga

SEQ ID NO: 69 - DNA, 37 bp, Artificial Sequence
  synthetic oligonucleotide
aacttgaatatatgggtnnsataagcgacagtggtat

SEQ ID NO: 70 - DNA, 37 bp, Artificial Sequence
  synthetic oligonucleotide
ataccactgtcgcttatsnnacccatatattcaagtt

SEQ ID NO: 71 - DNA, 37 bp, Artificial Sequence
  synthetic oligonucleotide
ttgaatatatgggttacnnsagcgacagtggtatcac

SEQ ID NO: 72 - DNA, 37 bp, Artificial Sequence
  synthetic oligonucleotide
gtgataccactgtcgctsnngtaacccatatattcaa

SEQ ID NO: 73 - DNA, 39 bp, Artificial Sequence
  synthetic oligonucleotide
gaatatatgggttacatannsgacagtggtatcacttac

SEQ ID NO: 74 - DNA, 39 bp, Artificial Sequence
  synthetic oligonucleotide
gtaagtgataccactgtcsnntatgtaacccatatattc

SEQ ID NO: 75 - DNA, 39 bp, Artificial Sequence
  synthetic oligonucleotide
tatatgggttacataagcnnsagtggtatcacttactac

SEQ ID NO: 76 - DNA, 39 bp, Artificial Sequence
  synthetic oligonucleotide
gtagtaagtgataccactsnngcttatgtaacccatata

SEQ ID NO: 77 - DNA, 39 bp, Artificial Sequence
  synthetic oligonucleotide
atgggttacataagcgacnnsggtatcacttactacaat

SEQ ID NO: 78 - DNA, 39 bp, Artificial Sequence
  synthetic oligonucleotide
attgtagtaagtgataccsnngtcgcttatgtaacccat

SEQ ID NO: 79 - DNA, 37 bp, Artificial Sequence
  synthetic oligonucleotide
gttacataagcgacagtnnsatcacttactacaatcc

SEQ ID NO: 80 - DNA, 37 bp, Artificial Sequence
  synthetic oligonucleotide
ggattgtagtaagtgatsnnactgtcgcttatgtaac

SEQ ID NO: 81 - DNA, 37 bp, Artificial Sequence
  synthetic oligonucleotide
acataagcgacagtggtnnsacttactacaatccatc

SEQ ID NO: 82 - DNA, 37 bp, Artificial Sequence
  synthetic oligonucleotide
gatggattgtagtaagtsnnaccactgtcgcttatgt

SEQ ID NO: 83 - DNA, 37 bp, Artificial Sequence
  synthetic oligonucleotide
taagcgacagtggtatcnnstactacaatccatctct

SEQ ID NO: 84 - DNA, 37 bp, Artificial Sequence
  synthetic oligonucleotide
agagatggattgtagtasnngataccactgtcgctta

SEQ ID NO: 85 - DNA, 43 bp, Artificial Sequence
  synthetic oligonucleotide
taagcgacagtggtatcactnnstacaatccatctctcaaaag

SEQ ID NO: 86 - DNA, 43 bp, Artificial Sequence
  synthetic oligonucleotide
cttttgagagatggattgtasnnagtgataccactgtcgctta

SEQ ID NO: 87 - DNA, 39 bp, Artificial Sequence
  synthetic oligonucleotide
gacagtggtatcacttacnnsaatccatctctcaaaagt

SEQ ID NO: 88 - DNA, 39 bp, Artificial Sequence
  synthetic oligonucleotide
acttttgagagatggattsnngtaagtgataccactgtc

SEQ ID NO: 89 - DNA, 37 bp, Artificial Sequence
  synthetic oligonucleotide
gtggtatcacttactacnnsccatctctcaaaagtcg

SEQ ID NO: 90 - DNA, 37 bp, Artificial Sequence
  synthetic oligonucleotide
cgacttttgagagatggsnngtagtaagtgataccac

SEQ ID NO: 91 - DNA, 37 bp, Artificial Sequence
  synthetic oligonucleotide
gtatcacttactacaatnnstctctcaaaagtcgcat

SEQ ID NO: 92 - DNA, 37 bp, Artificial Sequence
  synthetic oligonucleotide
atgcgacttttgagagasnnattgtagtaagtgatac

SEQ ID NO: 93 - DNA, 37 bp, Artificial Sequence
  synthetic oligonucleotide
tcacttactacaatccannsctcaaaagtcgcatttc

SEQ ID NO: 94 - DNA, 37 bp, Artificial Sequence
  synthetic oligonucleotide
gaaatgcgacttttgagsnntggattgtagtaagtga

SEQ ID NO: 95 - DNA, 37 bp, Artificial Sequence
  synthetic oligonucleotide
cttactacaatccatctnnsaaaagtcgcatttccat

SEQ ID NO: 96 - DNA, 37 bp, Artificial Sequence
  synthetic oligonucleotide
atggaaatgcgacttttsnnagatggattgtagtaag

SEQ ID NO: 97 - DNA, 37 bp, Artificial Sequence
  synthetic oligonucleotide
actacaatccatctctcnnsagtcgcatttccatcac

SEQ ID NO: 98 - DNA, 37 bp, Artificial Sequence
  synthetic oligonucleotide
gtgatggaaatgcgactsnngagagatggattgtagt

SEQ ID NO: 99 - DNA, 37 bp, Artificial Sequence
  synthetic oligonucleotide
acaatccatctctcaaannscgcatttccatcactcg

SEQ ID NO: 100 - DNA, 37 bp, Artificial Sequence
  synthetic oligonucleotide
cgagtgatggaaatgcgsnntttgagagatggattgt

SEQ ID NO: 101 - DNA, 39 bp, Artificial Sequence
  synthetic oligonucleotide
gccacatattactgtgcannsaggactctggctacttac

SEQ ID NO: 102 - DNA, 39 bp, Artificial Sequence
  synthetic oligonucleotide
gtaagtagccagagtcctsnntgcacagtaatatgtggc

SEQ ID NO: 103 - DNA, 37 bp, Artificial Sequence
  synthetic oligonucleotide
catattactgtgcaagannsactctggctacttacta

SEQ ID NO: 104 - DNA, 37 bp, Artificial Sequence
  synthetic oligonucleotide
tagtaagtagccagagtsnntcttgcacagtaatatg

SEQ ID NO: 105 - DNA, 37 bp, Artificial Sequence
  synthetic oligonucleotide
attactgtgcaagaaggnnsctggctacttactatgc

SEQ ID NO: 106 - DNA, 37 bp, Artificial Sequence
  synthetic oligonucleotide
gcatagtaagtagccagsnnccttcttgcacagtaat

SEQ ID NO: 107 - DNA, 37 bp, Artificial Sequence
  synthetic oligonucleotide
actgtgcaagaaggactnnsgctacttactatgctat

SEQ ID NO: 108 - DNA, 37 bp, Artificial Sequence
  synthetic oligonucleotide
atagcatagtaagtagcsnnagtccttcttgcacagt

SEQ ID NO: 109 - DNA, 37 bp, Artificial Sequence
  synthetic oligonucleotide
gtgcaagaaggactctgnnsacttactatgctatgga

SEQ ID NO: 110 - DNA, 37 bp, Artificial Sequence
  synthetic oligonucleotide
tccatagcatagtaagtsnncagagtccttcttgcac

SEQ ID NO: 111 - DNA, 37 bp, Artificial Sequence
  synthetic oligonucleotide
caagaaggactctggctnnstactatgctatggacta

SEQ ID NO: 112 - DNA, 37 bp, Artificial Sequence
  synthetic oligonucleotide
tagtccatagcatagtasnnagccagagtccttcttg

SEQ ID NO: 113 - DNA, 37 bp, Artificial Sequence
  synthetic oligonucleotide
gaaggactctggctactnnstatgctatggactactg

SEQ ID NO: 114 - DNA, 37 bp, Artificial Sequence
  synthetic oligonucleotide
cagtagtccatagcatasnnagtagccagagtccttc

SEQ ID NO: 115 - DNA, 37 bp, Artificial Sequence
  synthetic oligonucleotide
ggactctggctacttacnnsgctatggactactgggg

SEQ ID NO: 116 - DNA, 37 bp, Artificial Sequence
  synthetic oligonucleotide
ccccagtagtccatagcsnngtaagtagccagagtcc

SEQ ID NO: 117 - DNA, 37 bp, Artificial Sequence
  synthetic oligonucleotide
ctctggctacttactatnnsatggactactggggtca

SEQ ID NO: 118 - DNA, 37 bp, Artificial Sequence
  synthetic oligonucleotide
tgaccccagtagtccatsnnatagtaagtagccagag

SEQ ID NO: 119 - DNA, 37 bp, Artificial Sequence
  synthetic oligonucleotide
tggctacttactatgctnnsgactactggggtcaagg

SEQ ID NO: 120 - DNA, 37 bp, Artificial Sequence
  synthetic oligonucleotide
ccttgaccccagtagtcsnnagcatagtaagtagcca

SEQ ID NO: 121 - DNA, 37 bp, Artificial Sequence
  synthetic oligonucleotide
ctacttactatgctatgnnstactggggtcaaggaac

SEQ ID NO: 122 - DNA, 37 bp, Artificial Sequence
  synthetic oligonucleotide
gttccttgaccccagtasnncatagcatagtaagtag

SEQ ID NO: 123 - DNA, 37 bp, Artificial Sequence
  synthetic oligonucleotide
cttactatgctatggacnnstggggtcaaggaacctc

SEQ ID NO: 124 - DNA, 37 bp, Artificial Sequence
  synthetic oligonucleotide
gaggttccttgaccccasnngtccatagcatagtaag

SEQ ID NO: 125 - DNA, 37 bp, Artificial Sequence
  synthetic oligonucleotide
actatgctatggactacnnsggtcaaggaacctctgt

SEQ ID NO: 126 - DNA, 37 bp, Artificial Sequence
  synthetic oligonucleotide
acagaggttccttgaccsnngtagtccatagcatagt

SEQ ID NO: 127 - DNA, 37 bp, Artificial Sequence
  synthetic oligonucleotide
cctccatctcttgcaggnnsagtcagagccttgtaca

SEQ ID NO: 128 - DNA, 37 bp, Artificial Sequence
  synthetic oligonucleotide
tgtacaaggctctgactsnncctgcaagagatggagg

SEQ ID NO: 129 - DNA, 37 bp, Artificial Sequence
  synthetic oligonucleotide
ccatctcttgcagggctnnscagagccttgtacacag

SEQ ID NO: 130 - DNA, 37 bp, Artificial Sequence
  synthetic oligonucleotide
ctgtgtacaaggctctgsnnagccctgcaagagatgg

SEQ ID NO: 131 - DNA, 39 bp, Artificial Sequence
  synthetic oligonucleotide
atctcttgcagggctagtnnsagccttgtacacagtaat

SEQ ID NO: 132 - DNA, 39 bp, Artificial Sequence
  synthetic oligonucleotide
attactgtgtacaaggctsnnactagccctgcaagagat

SEQ ID NO: 133 - DNA, 37 bp, Artificial Sequence
  synthetic oligonucleotide
cttgcagggctagtcagnnscttgtacacagtaatgg

SEQ ID NO: 134 - DNA, 37 bp, Artificial Sequence
  synthetic oligonucleotide
ccattactgtgtacaagsnnctgactagccctgcaag

SEQ ID NO: 135 - DNA, 39 bp, Artificial Sequence
  synthetic oligonucleotide
tgcagggctagtcagagcnnsgtacacagtaatggaaac

SEQ ID NO: 136 - DNA, 39 bp, Artificial Sequence
  synthetic oligonucleotide
gtttccattactgtgtacsnngctctgactagccctgca

SEQ ID NO: 137 - DNA, 37 bp, Artificial Sequence
  synthetic oligonucleotide
gggctagtcagagccttnnscacagtaatggaaacac

SEQ ID NO: 138 - DNA, 37 bp, Artificial Sequence
  synthetic oligonucleotide
gtgtttccattactgtgsnnaaggctctgactagccc

SEQ ID NO: 139 - DNA, 37 bp, Artificial Sequence
  synthetic oligonucleotide
ctagtcagagccttgtannsagtaatggaaacaccta

SEQ ID NO: 140 - DNA, 37 bp, Artificial Sequence
  synthetic oligonucleotide
taggtgtttccattactsnntacaaggctctgactag

SEQ ID NO: 141 - DNA, 41 bp, Artificial Sequence
  synthetic oligonucleotide
tagtcagagccttgtacacnnsaatggaaacacctatttac

SEQ ID NO: 142 - DNA, 41 bp, Artificial Sequence
  synthetic oligonucleotide
gtaaataggtgtttccattsnngtgtacaaggctctgacta

SEQ ID NO: 143 - DNA, 37 bp, Artificial Sequence
  synthetic oligonucleotide
agagccttgtacacagtnnsggaaacacctatttaca

SEQ ID NO: 144 - DNA, 37 bp, Artificial Sequence
  synthetic oligonucleotide
tgtaaataggtgtttccsnnactgtgtacaaggctct

SEQ ID NO: 145 - DNA, 37 bp, Artificial Sequence
  synthetic oligonucleotide
gccttgtacacagtaatnnsaacacctatttacattg

SEQ ID NO: 146 - DNA, 37 bp, Artificial Sequence
  synthetic oligonucleotide
caatgtaaataggtgttsnnattactgtgtacaaggc

SEQ ID NO: 147 - DNA, 37 bp, Artificial Sequence
  synthetic oligonucleotide
ttgtacacagtaatggannsacctatttacattggta

SEQ ID NO: 148 - DNA, 37 bp, Artificial Sequence
  synthetic oligonucleotide
taccaatgtaaataggtsnntccattactgtgtacaa

SEQ ID NO: 149 - DNA, 36 bp, Artificial Sequence
  synthetic oligonucleotide
tacacagtaatggaaacnnstatttacattggtacc

SEQ ID NO: 150 - DNA, 36 bp, Artificial Sequence
  synthetic oligonucleotide
ggtaccaatgtaaatasnngtttccattactgtgta

SEQ ID NO: 151 - DNA, 37 bp, Artificial Sequence
  synthetic oligonucleotide
acagtaatggaaacaccnnsttacattggtacctgca

SEQ ID NO: 152 - DNA, 37 bp, Artificial Sequence
  synthetic oligonucleotide
tgcaggtaccaatgtaasnnggtgtttccattactgt

SEQ ID NO: 153 - DNA, 39 bp, Artificial Sequence
  synthetic oligonucleotide
agtaatggaaacacctatnnscattggtacctgcagaag

SEQ ID NO: 154 - DNA, 39 bp, Artificial Sequence
  synthetic oligonucleotide
cttctgcaggtaccaatgsnnataggtgtttccattact

SEQ ID NO: 155 - DNA, 37 bp, Artificial Sequence
  synthetic oligonucleotide
atggaaacacctatttannstggtacctgcagaagcc

SEQ ID NO: 156 - DNA, 37 bp, Artificial Sequence
  synthetic oligonucleotide
ggcttctgcaggtaccasnntaaataggtgtttccat

SEQ ID NO: 157 - DNA, 37 bp, Artificial Sequence
  synthetic oligonucleotide
ctccaaagctcctgatcnnsagagtttccaaccgatt

SEQ ID NO: 158 - DNA, 37 bp, Artificial Sequence
  synthetic oligonucleotide
aatcggttggaaactctsnngatcaggagctttggag

SEQ ID NO: 159 - DNA, 37 bp, Artificial Sequence
  synthetic oligonucleotide
caaagctcctgatctacnnsgtttccaaccgattttc

SEQ ID NO: 160 - DNA, 37 bp, Artificial Sequence
  synthetic oligonucleotide
gaaaatcggttggaaacsnngtagatcaggagctttg

SEQ ID NO: 161 - DNA, 37 bp, Artificial Sequence
  synthetic oligonucleotide
agctcctgatctacagannstccaaccgattttctgg

SEQ ID NO: 162 - DNA, 37 bp, Artificial Sequence
  synthetic oligonucleotide
ccagaaaatcggttggasnntctgtagatcaggagct

SEQ ID NO: 163 - DNA, 37 bp, Artificial Sequence
  synthetic oligonucleotide
tcctgatctacagagttnnsaaccgattttctggggt

SEQ ID NO: 164 - DNA, 37 bp, Artificial Sequence
  synthetic oligonucleotide
accccagaaaatcggttsnnaactctgtagatcagga

SEQ ID NO: 165 - DNA, 37 bp, Artificial Sequence
  synthetic oligonucleotide
tgatctacagagtttccnnscgattttctggggtccc

SEQ ID NO: 166 - DNA, 37 bp, Artificial Sequence
  synthetic oligonucleotide
gggaccccagaaaatcgsnnggaaactctgtagatca

SEQ ID NO: 167 - DNA, 37 bp, Artificial Sequence
  synthetic oligonucleotide
tctacagagtttccaacnnsttttctggggtcccaga

SEQ ID NO: 168 - DNA, 37 bp, Artificial Sequence
  synthetic oligonucleotide
tctgggaccccagaaaasnngttggaaactctgtaga

SEQ ID NO: 169 - DNA, 37 bp, Artificial Sequence
  synthetic oligonucleotide
acagagtttccaaccgannstctggggtcccagacag

SEQ ID NO: 170 - DNA, 37 bp, Artificial Sequence
  synthetic oligonucleotide
ctgtctgggaccccagasnntcggttggaaactctgt

SEQ ID NO: 171 - DNA, 37 bp, Artificial Sequence
  synthetic oligonucleotide
gagtttccaaccgatttnnsggggtcccagacaggtt

SEQ ID NO: 172 - DNA, 37 bp, Artificial Sequence
  synthetic oligonucleotide
aacctgtctgggaccccsnnaaatcggttggaaactc

SEQ ID NO: 173 - DNA, 37 bp, Artificial Sequence
  synthetic oligonucleotide
gagtttatttctgctctnnsagtacacatgttcctcc

SEQ ID NO: 174 - DNA, 37 bp, Artificial Sequence
  synthetic oligonucleotide
ggaggaacatgtgtactsnnagagcagaaataaactc

SEQ ID NO: 175 - DNA, 43 bp, Artificial Sequence
  synthetic oligonucleotide
gagtttatttctgctctcaannsacacatgttcctccgcattt

SEQ ID NO: 176 - DNA, 43 bp, Artificial Sequence
  synthetic oligonucleotide
aaatgcggaggaacatgtgtsnnttgagagcagaaataaactc

SEQ ID NO: 177 - DNA, 39 bp, Artificial Sequence
  synthetic oligonucleotide
tatttctgctctcaaagtnnscatgttcctccgcatttc

SEQ ID NO: 178 - DNA, 39 bp, Artificial Sequence
  synthetic oligonucleotide
gaaatgcggaggaacatgsnnactttgagagcagaaata

SEQ ID NO: 179 - DNA, 37 bp, Artificial Sequence
  synthetic oligonucleotide
tctgctctcaaagtacannsgttcctccgcatttcgg

SEQ ID NO: 180 - DNA, 37 bp, Artificial Sequence
  synthetic oligonucleotide
ccgaaatgcggaggaacsnntgtactttgagagcaga

SEQ ID NO: 181 - DNA, 37 bp, Artificial Sequence
  synthetic oligonucleotide
gctctcaaagtacacatnnscctccgcatttcggtgg

SEQ ID NO: 182 - DNA, 37 bp, Artificial Sequence
  synthetic oligonucleotide
ccaccgaaatgcggaggsnnatgtgtactttgagagc

SEQ ID NO: 183 - DNA, 37 bp, Artificial Sequence
  synthetic oligonucleotide
ctcaaagtacacatgttnnsccgcatttcggtggagg

SEQ ID NO: 184 - DNA, 37 bp, Artificial Sequence
  synthetic oligonucleotide
cctccaccgaaatgcggsnnaacatgtgtactttgag

SEQ ID NO: 185 - DNA, 39 bp, Artificial Sequence
  synthetic oligonucleotide
caaagtacacatgttcctnnscatttcggtggaggcacc

SEQ ID NO: 186 - DNA, 39 bp, Artificial Sequence
  synthetic oligonucleotide
ggtgcctccaccgaaatgsnnaggaacatgtgtactttg

SEQ ID NO: 187 - DNA, 39 bp, Artificial Sequence
  synthetic oligonucleotide
agtacacatgttcctccgnnsttcggtggaggcaccaag

SEQ ID NO: 188 - DNA, 39 bp, Artificial Sequence
  synthetic oligonucleotide
cttggtgcctccaccgaasnncggaggaacatgtgtact

SEQ ID NO: 189 - DNA, 37 bp, Artificial Sequence
  synthetic oligonucleotide
cggccatggcccaggtgcagctgcagcagtctggggc

SEQ ID NO: 190 - DNA, 37 bp, Artificial Sequence
  synthetic oligonucleotide
ctggggcagaacttgtgaaatcagggacctcagtcaa

SEQ ID NO: 191 - DNA, 37 bp, Artificial Sequence
  synthetic oligonucleotide
gggcagaacttgtgaggccggggacctcagtcaagtt

SEQ ID NO: 192 - DNA, 37 bp, Artificial Sequence
  synthetic oligonucleotide
aacttgtgaggtcagggggctcagtcaagttgtcctg

SEQ ID NO: 193 - DNA, 37 bp, Artificial Sequence
  synthetic oligonucleotide
gcacagcttctggcttcaccattaaagactcctatat

SEQ ID NO: 194 - DNA, 37 bp, Artificial Sequence
  synthetic oligonucleotide
cagcttctggcttcaactttaaagactcctatatgca

SEQ ID NO: 195 - DNA, 37 bp, Artificial Sequence
  synthetic oligonucleotide
cttctggcttcaacattagcgactcctatatgcactg

SEQ ID NO: 196 - DNA, 37 bp, Artificial Sequence
  synthetic oligonucleotide
actcctatatgcactgggtgaggcaggggcctgaaca

SEQ ID NO: 197 - DNA, 37 bp, Artificial Sequence
  synthetic oligonucleotide
tgcactggttgaggcaggcgcctgaacagggcctgga

SEQ ID NO: 198 - DNA, 37 bp, Artificial Sequence
  synthetic oligonucleotide
ggttgaggcaggggcctggccagggcctggagtggat

SEQ ID NO: 199 - DNA, 37 bp, Artificial Sequence
  synthetic oligonucleotide
ccccgaagttccagggccgtgccacttttactacaga

SEQ ID NO: 200 - DNA, 37 bp, Artificial Sequence
  synthetic oligonucleotide
cgaagttccagggcaagttcacttttactacagacac

SEQ ID NO: 201 - DNA, 37 bp, Artificial Sequence
  synthetic oligonucleotide
tccagggcaaggccactattactacagacacatcctc

SEQ ID NO: 202 - DNA, 37 bp, Artificial Sequence
  synthetic oligonucleotide
gcaaggccacttttactcgcgacacatcctccaacac

SEQ ID NO: 203 - DNA, 37 bp, Artificial Sequence
  synthetic oligonucleotide
ttactacagacacatccaaaaacacagcctacctgca

SEQ ID NO: 204 - DNA, 37 bp, Artificial Sequence
  synthetic oligonucleotide
ctgccgtctattattgtgcggaggggactccgactgg

SEQ ID NO: 205 - DNA, 37 bp, Artificial Sequence
  synthetic oligonucleotide
ccgtctattattgtaatcgcgggactccgactgggcc

SEQ ID NO: 206 - DNA, 37 bp, Artificial Sequence
  synthetic oligonucleotide
ctggcggtggcggatcacagaatgtgctcacccagtc

SEQ ID NO: 207 - DNA, 37 bp, Artificial Sequence
  synthetic oligonucleotide
gcggtggcggatcagaaagcgtgctcacccagtctcc

SEQ ID NO: 208 - DNA, 38 bp, Artificial Sequence
  synthetic oligonucleotide
gaaaatgtgctcacccagccgccagcaatcatgtctgc

SEQ ID NO: 209 - DNA, 37 bp, Artificial Sequence
  synthetic oligonucleotide
tgctcacccagtctccaagcatcatgtctgcatctcc

SEQ ID NO: 210 - DNA, 37 bp, Artificial Sequence
  synthetic oligonucleotide
cccagtctccagcaatcgtgtctgcatctccagggga

SEQ ID NO: 211 - DNA, 37 bp, Artificial Sequence
  synthetic oligonucleotide
tgtctgcatctccagggcagaaggtcaccataacctg

SEQ ID NO: 212 - DNA, 37 bp, Artificial Sequence
  synthetic oligonucleotide
ctgcatctccaggggagaccgtcaccataacctgcag

SEQ ID NO: 213 - DNA, 37 bp, Artificial Sequence
  synthetic oligonucleotide
taagttacatgcactggtaccagcagaagccaggcac

SEQ ID NO: 214 - DNA, 37 bp, Artificial Sequence
  synthetic oligonucleotide
gcacttctcccaaactcgtgatttatagcacatccaa

SEQ ID NO: 215 - DNA, 37 bp, Artificial Sequence
  synthetic oligonucleotide
tggcttctggagtccctgatcgcttcagtggcagtgg

SEQ ID NO: 216 - DNA, 37 bp, Artificial Sequence
  synthetic oligonucleotide
ctcgcttcagtggcagtaaatctgggacctcttactc

SEQ ID NO: 217 - DNA, 37 bp, Artificial Sequence
  synthetic oligonucleotide
gtggatctgggacctctgcgtctctcacaatcagccg

SEQ ID NO: 218 - DNA, 37 bp, Artificial Sequence
  synthetic oligonucleotide
ctctcacaatcagccgactggaggctgaagatgctgc

SEQ ID NO: 219 - DNA, 37 bp, Artificial Sequence
  synthetic oligonucleotide
gaatggaggctgaagatgaagccacttattactgcca

SEQ ID NO: 220 - DNA, 37 bp, Artificial Sequence
  synthetic oligonucleotide
aggctgaagatgctgccgattattactgccagcaaag

SEQ ID NO: 221 - DNA, 37 bp, Artificial Sequence
  synthetic oligonucleotide
acccactcacgttcggtggcggcaccaagctggagct

SEQ ID NO: 222 - DNA, 37 bp, Artificial Sequence
  primer
cttctggcttcaacattsatgactcctatatgcactg

SEQ ID NO: 223 - DNA, 37 bp, Artificial Sequence
  primer
ctggcttcaacattaaasattcctatatgcactgggt

SEQ ID NO: 224 - DNA, 37 bp, Artificial Sequence
  primer
gcttcaacattaaagacsattatatgcactgggtgag

SEQ ID NO: 225 - DNA, 37 bp, Artificial Sequence
  primer
tcaacattaaagactccsatatgcactgggtgaggca

SEQ ID NO: 226 - DNA, 37 bp, Artificial Sequence
  primer
ttaaagactcctatatgsattgggtgaggcaggggcc

SEQ ID NO: 227 - DNA, 37 bp, Artificial Sequence
  primer
gcctggagtggattggasatattgatcctgagaatgg

SEQ ID NO: 228 - DNA, 37 bp, Artificial Sequence
  primer
agtggattggatggattsatcctgagaatggtgatac

SEQ ID NO: 229 - DNA, 37 bp, Artificial Sequence
  primer
ttggatggattgatcctsataatggtgatactgaata

SEQ ID NO: 230 - DNA, 37 bp, Artificial Sequence
  primer
gatggattgatcctgagsatggtgatactgaatatgc

SEQ ID NO: 231 - DNA, 37 bp, Artificial Sequence
  primer
ttgatcctgagaatggtsatactgaatatgccccgaa

SEQ ID NO: 232 - DNA, 37 bp, Artificial Sequence
  primer
atcctgagaatggtgatsatgaatatgccccgaagtt

SEQ ID NO: 233 - DNA, 37 bp, Artificial Sequence
  primer
ctgagaatggtgatactsattatgccccgaagttcca

SEQ ID NO: 234 - DNA, 37 bp, Artificial Sequence
  primer
gtgatactgaatatgccsataagttccagggcaaggc

SEQ ID NO: 235 - DNA, 37 bp, Artificial Sequence
  primer
atactgaatatgccccgsatttccagggcaaggccac

SEQ ID NO: 236 - DNA, 37 bp, Artificial Sequence
  primer
aatatgccccgaagttcsatggcaaggccacttttac

SEQ ID NO: 237 - DNA, 37 bp, Artificial Sequence
  primer
ccgtctattattgtaatsatgggactccgactgggcc

SEQ ID NO: 238 - DNA, 37 bp, Artificial Sequence
  primer
tctattattgtaatgagsatactccgactgggccgta

SEQ ID NO: 239 - DNA, 37 bp, Artificial Sequence
  primer
attattgtaatgaggggsatccgactgggccgtacta

SEQ ID NO: 240 - DNA, 37 bp, Artificial Sequence
  primer
attgtaatgaggggactsatactgggccgtactactt

SEQ ID NO: 241 - DNA, 37 bp, Artificial Sequence
  primer
gtaatgaggggactccgsatgggccgtactactttga

SEQ ID NO: 242 - DNA, 37 bp, Artificial Sequence
  primer
atgaggggactccgactsatccgtactactttgacta

SEQ ID NO: 243 - DNA, 37 bp, Artificial Sequence
  primer
aggggactccgactgggsattactactttgactactg

SEQ ID NO: 244 - DNA, 37 bp, Artificial Sequence
  primer
ctccgactgggccgtacsattttgactactggggcca

SEQ ID NO: 245 - DNA, 37 bp, Artificial Sequence
  primer
taacctgcagtgccagcsatagtgtaagttacatgca

SEQ ID NO: 246 - DNA, 37 bp, Artificial Sequence
  primer
cctgcagtgccagctcasatgtaagttacatgcactg

SEQ ID NO: 247 - DNA, 37 bp, Artificial Sequence
  primer
gcagtgccagctcaagtsatagttacatgcactggtt

SEQ ID NO: 248 - DNA, 37 bp, Artificial Sequence
  primer
gtgccagctcaagtgtasattacatgcactggttcca

SEQ ID NO: 249 - DNA, 37 bp, Artificial Sequence
  primer
ccagctcaagtgtaagtsatatgcactggttccagca

SEQ ID NO: 250 - DNA, 37 bp, Artificial Sequence
  primer
ctcccaaactcgtgattsatagcacatccaacctggc

SEQ ID NO: 251 - DNA, 37 bp, Artificial Sequence
  primer
ccaaactcgtgatttatsatacatccaacctggcttc

SEQ ID NO: 252 - DNA, 37 bp, Artificial Sequence
  primer
aactcgtgatttatagcsattccaacctggcttctgg

SEQ ID NO: 253 - DNA, 37 bp, Artificial Sequence
  primer
tcgtgatttatagcacasataacctggcttctggagt

SEQ ID NO: 254 - DNA, 37 bp, Artificial Sequence
  primer
tgatttatagcacatccsatctggcttctggagtccc

SEQ ID NO: 255 - DNA, 37 bp, Artificial Sequence
  primer
atagcacatccaacctgsattctggagtccctgctcg

SEQ ID NO: 256 - DNA, 37 bp, Artificial Sequence
  primer
gcacatccaacctggctsatggagtccctgctcgctt

SEQ ID NO: 257 - DNA, 37 bp, Artificial Sequence
  primer
cttattactgccagcaasattctagttacccactcac

SEQ ID NO: 258 - DNA, 36 bp, Artificial Sequence
  primer
attactgccagcaaagasatagttacccactcacgt

SEQ ID NO: 259 - DNA, 36 bp, Artificial Sequence
  primer
actgccagcaaagatctsattacccactcacgttcg

SEQ ID NO: 260 - DNA, 36 bp, Artificial Sequence
  primer
gccagcaaagatctagtsatccactcacgttcggtg

SEQ ID NO: 261 - DNA, 37 bp, Artificial Sequence
  primer
aaagatctagttacccasatacgttcggtgctggcac

SEQ ID NO: 262 - DNA, 17 bp, Artificial Sequence
  primer
caggaaacagctatgac

SEQ ID NO: 263 - DNA, 22 bp, Artificial Sequence
  primer
ggaccacggtcaccgtctcctc